(19) United States
(12) Patent Application Publication
(10) Pub. No.: US 2024/0173391 A1
(43) Pub. Date: May 30, 2024

(54) THERAPEUTIC COMBINATION FOR TREATING CANCER

(71) Applicants: Nykode Therapeutics ASA, Oslo (NO); Genentech Inc., South San Francisco, CA (US)

(72) Inventors: Agnete Øvre Fredriksen, Rælingen (NO); Karoline Schjetne, Lommedalen (NO); Stine Granum, Oslo (NO); Audun Trygge Haugen Bersaas, Oslo (NO); Ina Park Rhee, South San Francisco, CA (US); Siri Brinchmann-Hansen Torhaug, Stabekk (NO)

(73) Assignee: Nykode Therapeutics ASA, Oslo (NO)

(21) Appl. No.: 18/552,383

(22) PCT Filed: Mar. 25, 2022
(86) PCT No.: PCT/EP2022/057955

§ 371 Date: Sep. 25, 2023

(30) Foreign Application Priority Data
Mar. 26, 2021 (DK) PA 2021 00317
Apr. 21, 2021 (DK) PA 2021 70182

(51) Int. Cl. A61K 39/00; A61K 39/395; A61K 45/06; A61P 35/00; C07K 16/28

(52) U.S. Cl. CPC A61K 39/0011; A61K 39/39541; A61K 45/06; A61P 35/00; C07K 16/2803; C07K 16/2818; C07K 16/2827; A61K 2039/505; A61K 2039/53; A61K 2039/6056; A61K 2039/627

(57) ABSTRACT

This invention relates to methods and kits for treating a subject having cancer, e.g. a patient, by administering to the subject an anticancer vaccine in combination with one or more checkpoint inhibitors.

### TECHNICAL FIELD

This invention relates to methods and kits for treating a subject having cancer, e.g. a patient, by administering to the subject an anticancer vaccine in combination with one or more checkpoint inhibitors.

### BACKGROUND

Although treatment of cancer has been improved over the past few decades in particular due to early detection and diagnosis, which has significantly increased survival, only about 60% of patients diagnosed with cancer are alive 5 years after the diagnosis. Current cancer treatments include, in addition to surgical removal of tumors and lesions, radiation and cytotoxic chemotherapeutics, each of which has its own well-known advantages and drawbacks.

More recently, immunotherapies have become an alternative approach for treating cancer. Therapeutic anticancer vaccines represent a class of immunotherapy that work by stimulating or restoring the ability of a subject's immune system's ability to fight cancer. Therapeutic anticancer vaccines, as opposed to preventative or prophylactic vaccines, are used to treat an existing cancer. Anticancer vaccines may reduce or even eliminate some of the side effects seen with traditional cancer treatments.

One class of such anticancer vaccines are those developed by the applicant. These are polypeptides—or polynucleotides encoding such polypeptides—comprising three subunits: a targeting unit that targets antigen-presenting cells (APCs), a dimerization unit and an antigenic unit that comprises one or more cancer antigens. Due to the dimerization unit, the polypeptides form a dimeric protein. Such anticancer vaccines are disclosed in e.g. WO 2004/076489A1, WO 2011/161244A1, WO 2013/092875A1 and WO 2017/118695A1.

When administered as a dimeric protein to a patient (or administered as a polynucleotide to a patient and expressed in vivo), the dimeric proteins target APCs, which results in enhanced vaccine potency compared to identical non-targeted antigens.

Another type of cancer immunotherapy are checkpoint inhibitors which target different immune checkpoints, i.e. key regulators of the immune system that when stimulated can dampen the immune response to an immunologic stimulus. Some cancers can protect themselves from attacks by the body's immune system by stimulating immune targets, thus corrupting or even nullifying the treatment success of anticancer immunotherapy. Checkpoint inhibitor therapy has been used to block inhibitory checkpoints and thereby restoring immune system function and treat cancers, e.g. melanoma or non-small cell lung carcinoma patients. There remains a need for effective cancer therapies.

### SUMMARY

This invention relates to methods and kits for treating a subject having cancer, e.g. a patient, by administering to the subject a certain anticancer vaccine in combination with one or more checkpoint inhibitors.

### DETAILED DESCRIPTION

Thus, in a first aspect, the invention provides a method for treating a subject having cancer, the method comprising administering to the subject
(a) an anticancer vaccine comprising
   (i) a polynucleotide comprising a nucleotide sequence encoding a targeting unit that targets antigen-presenting cells, a multimerization unit, such as a dimerization unit and an antigenic unit comprising one or more cancer antigens or parts thereof; or
   (ii) a polypeptide encoded by the polynucleotide as defined in (i); or
   (iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject having cancer, the method comprising administering to the subject
(a) an anticancer vaccine comprising:
(i) a polynucleotide comprising a nucleotide sequence encoding a targeting unit that targets antigen-presenting cells, a multimerization unit and an antigenic unit comprising one or more cancer antigens or parts thereof; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a method for treating a subject having cancer, the method comprising administering to the subject
(a) an anticancer vaccine comprising:
(i) a polynucleotide comprising a nucleotide sequence encoding a targeting unit that targets antigen-presenting cells, a dimerization unit and an antigenic unit comprising one or more cancer antigens or parts thereof; or
(ii) a polypeptide encoded by the polynucleotide as defined in (i); or
(iii) a dimeric protein consisting of two polypeptides as defined in (ii); and
(b) one or more checkpoint inhibitors.

A “subject” as used herein is an animal, e.g., a mouse, or human, preferably a human. The terms “subject having cancer” and “patient” may be used herein interchangeably.

A “cancer” refers to a broad group of various diseases characterized by the uncontrolled growth of abnormal cells in the body. A “cancer” or “cancer tissue” includes a tumor, and as used herein, encompasses both a solid tumor as well as tumor cells found in a bodily fluid such as blood, and includes metastatic cancer. Unregulated cell division and growth results in the formation of malignant tumors that can invade neighboring tissues and can also metastasize to distant parts of the body through the lymphatic system or bloodstream. Following metastasis, the distal tumors can be said to be “derived from” a pre-metastasis tumor.

A “cancer antigen” includes neoantigens, patient-present shared cancer antigens or shared cancer antigens, as described herein.

A “part” refers to a part/fragment of a cancer antigen, i.e. part/fragment of the amino acid sequence of a cancer antigen, or the nucleotide sequence encoding same, e.g. an epitope.

The section headings used herein are for organizational purposes only and are not to be construed as limiting the subject matter described.

### Anticancer Vaccine

The anticancer vaccine for use in the method according to the invention may be an individualized anticancer vaccine or a non-individualized anticancer vaccine, i.e. “off-the-shelf” anticancer vaccine (referred to as “shared anticancer vaccine”). These two types of anticancer vaccines differ in their antigenic units, as detailed below.

The anticancer vaccine for use in the method of the invention can be described as a polypeptide having an N-terminal start and a C-terminal end (illustrated in FIG. 1). The elements and units of the polypeptide—targeting unit (TU), multimerization unit, such as, in this Figure, a dimerization unit (DimU), and antigenic unit—may be arranged in the polypeptide such that the antigenic unit is located at the C-terminal end of the polypeptide (FIG. 1a) or at the N-terminal start of the polypeptide (FIG. 1b). Preferably, the antigenic unit is located at the C-terminal end of the polypeptide. A unit linker (UL) may connect the multimerization unit, such as a dimerization unit, and the antigenic unit. FIG. 1 illustrates an antigenic unit with 4 neoepitopes (neo1, neo2, neo3, neo4), which are separated by linkers (SUL1, SUL2, SUL3). An alternative way to describe the arrangement of the neoepitopes neo1-neo4 is that these neoepitopes are arranged in 3 antigenic subunits, each comprising a neoepitope and a subunit linker (SUL1, SUL2, SUL3), and a terminal neoepitope (neo4), which is closest to the C-terminal end or N-terminal start of the polypeptide. The subunits are indicated in the Figure by square brackets. Thus, an antigenic unit comprising n neoepitopes comprises n−1 subunits, each subunit comprising a neoepitope and a subunit linker. As described herein, the 4 neoepitopes may be identical or different neoepitopes and the 3 linkers/subunit linkers may be identical or different. The order and orientation of the above-described units and elements is the same in the protein and the polynucleotide.

In the following, the various units and elements of the anticancer vaccine will be discussed in detail. These units and elements are present in the polynucleotide as nucleic acid sequences encoding the units while they are present in the polypeptide or multimeric protein as amino acid sequences. For the ease of reading, in the following, the units of the anticancer vaccine are mainly explained in relation to the polypeptide/multimeric protein, i.e. on the basis of the amino acid sequences of such vaccines.

### Antigenic Unit

### Antigenic Unit of Individualized Anticancer Vaccines

An individualized anticancer vaccine for use in the method according to the invention comprises an antigenic unit, which is designed specifically, and only for the patient who is treated or who is to be treated with the vaccine. Thus, the antigenic unit of such a vaccine comprises one or more cancer antigens that are patient-specific antigens or parts thereof, such antigens including neoantigens or patient-present shared cancer antigens.

“Patient-present shared cancer antigen” is used herein to describe a shared cancer antigen or shared tumor antigen that has been identified to be present in the patient's tumor cells.

“Neoantigen or patient-specific cancer antigen” is used herein to describe a cancer antigen or tumor antigen found in a patient's tumor cells that comprises one or more mutations compared to the same patient's normal (i.e. healthy, non-cancerous) cells.

“Patient-present shared cancer epitope” is used herein to describe an amino acid sequence, or a nucleotide sequence encoding same, comprised in a patient-present shared cancer antigen, which comprises one or more immunogenic mutations, i.e. mutations which are known to be immunogenic or which are predicted to be immunogenic.

“Neoepitope or patient-specific cancer epitope” is used herein to describe an amino acid sequence, or a nucleotide sequence encoding same, comprised in a neoantigen or in a patient-specific cancer antigen, which comprises one or more immunogenic mutations, i.e. which are predicted to be immunogenic.

Thus, in one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit, such as a dimerization unit and an antigenic unit, wherein the antigenic unit comprises one or more patient-specific cancer antigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit and an antigenic unit, wherein the antigenic unit comprises one or more patient-specific cancer antigens or parts thereof; or
(ii) a polypeptide encoded by the polynucleotide as defined in (i); or
(iii) a multimeric protein, consisting of multiple polypeptides as defined in (ii); and
(b) one or more checkpoint inhibitors.

In another embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a dimerization unit and an antigenic unit, wherein the antigenic unit comprises one or more patient-specific cancer antigens or parts thereof; or
(ii) a polypeptide encoded by the polynucleotide as defined in (i); or
(iii) a dimeric protein consisting of two polypeptides as defined in (ii); and
(b) one or more checkpoint inhibitors.

In one embodiment, the patient-specific cancer antigen is a patient-present shared cancer antigen. Thus, in such embodiment, the antigenic unit comprises one or more patient-present shared cancer antigens or parts thereof, e.g. one patient-present shared cancer antigen or one or more parts of such one patient-present shared cancer antigen, e.g. one or more epitopes, or several patient-present shared cancer antigens or one or more parts of several patient-present shared cancer antigens, e.g. one or more epitopes.

In another embodiment, the patient-specific cancer antigen is a neoantigen. Thus, in such embodiment, the antigenic unit comprises one or more neoantigens or parts thereof, e.g. one neoantigen or one or more parts of such one neoantigen, e.g. one or more neoepitopes, or several neoantigens or one or more parts of several neoantigens, e.g. one or more neoepitopes.

In yet another embodiment, the antigenic unit comprises any combinations of the aforementioned embodiments, i.e. any combination of one or more patient-present shared cancer antigens or parts thereof and of one or more neoantigens or parts thereof mentioned above.

### Antigenic Unit of Individualized Anticancer Vaccines Comprising One or More Neoantigens or Parts Thereof

Cancers develop from the patient's normal tissue by one or a few cells starting an abnormal, uncontrolled proliferation of the cells due to mutations. Although the cancer cells are mutated, most of the genome in those cells is intact and identical to the remaining cells in the patient. One approach of attacking a tumor is based on the knowledge that any tumor in any patient is unique: patient-specific mutations lead to expression of patient-specific mutated proteins, i.e. patient-specific cancer antigens, i.e. neoantigens, that are unique for the particular patient. These neoantigens are not identical to any proteins in the normal cells of the patient. Therefore, such neoantigens are suitable targets for a therapeutic anticancer vaccine which is manufactured specifically and only for the patient in question, i.e. an individualized anticancer vaccine.

The mutation may be any mutation leading to a change in at least one amino acid. Accordingly, the mutation may be one of the following:
a non-synonymous mutation leading to a change in the amino acid
a mutation leading to a frame shift and thereby a completely different open reading frame in the direction after the mutation
a read-through mutation in which a stop codon is modified or deleted leading to a longer protein with a tumor-specific epitope
splice mutations that lead to a unique tumor-specific protein sequence
chromosomal rearrangements that give rise to a chimeric protein with a tumor-specific epitope at the junction of the two proteins. When the mutation is due to a chromosomal rearrangement, the tumor-specific epitope can arise from a change in at least one amino acid or from a combination of two in-frame coding sequences.

In one embodiment, the antigenic unit comprises one or more neoantigens or parts thereof, preferably one or more neoepitopes and more preferably several neoepitopes. Such neoepitopes may be selected for inclusion into antigenic unit according to their predicted therapeutic efficacy, see WO 2017/118695A1, the disclosures of which is incorporated herein by reference.

Thus, in one embodiment, the invention provides a method for treating a subject (e.g. a patient) having cancer, the method comprising administering to the subject
(a) an anticancer vaccine comprising
(i) a polynucleotide comprising a nucleotide sequence encoding a targeting unit that targets antigen-presenting cells, a multimerization unit, such as a dimerization unit, and an antigenic unit, wherein the antigenic unit comprises one or more neoantigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject (e.g. a patient) having cancer, the method comprising administering to the subject
(a) an anticancer vaccine comprising
(i) a polynucleotide comprising a nucleotide sequence encoding a targeting unit that targets antigen-presenting cells, multimerization unit and an antigenic unit, wherein the antigenic unit comprises one or more neoantigens or parts thereof; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a method for treating a subject (e.g. a patient) having cancer, the method comprising administering to the subject
(a) an anticancer vaccine comprising
(i) a polynucleotide comprising a nucleotide sequence encoding a targeting unit that targets antigen-presenting cells, a dimerization unit and an antigenic unit, wherein the antigenic unit comprises one or more neoantigens or parts thereof; or
(ii) a polypeptide encoded by the polynucleotide as defined in (i); or
(iii) a dimeric protein consisting of two polypeptides as defined in (ii); and
(b) one or more checkpoint inhibitors.

In one embodiment, the antigenic unit comprises one or more parts of one neoantigen or one or more parts of several neoantigens, preferably one or more neoepitopes. In a preferred embodiment, in the antigenic unit, the neoepitopes are separated by linkers. An alternative way to describe the separation of all neoepitopes by linkers is that all but the terminal neoepitope, i.e. the neoepitope at the N-terminal start of the polypeptide or the C-terminal end of the polypeptide, are arranged in antigenic subunits, wherein each subunit comprises a neoepitope and a subunit linker. Due to the separation of the neoepitopes by a linker, each neoepitope is presented in an optimal way to the immune system.

Hence, an antigenic unit comprising n neoepitopes comprises n−1 antigenic subunits, wherein each subunit comprises a neoepitope and a subunit linker, and further comprises a terminal neoepitope. In one embodiment n is an integer of from 1 to 50, e.g. 3 to 50 or 15 to 40 or 10 to 30 or 10 to 25 or 10 to 20 or 15 to 30 or 15 to 25 or 15 to 20. In a preferred embodiment, the antigenic subunit consists of a neoepitope and a subunit linker.

Thus, in one embodiment, the invention provides a method for treating a subject having cancer, the method comprising administering to the subject:
(a) an anticancer vaccine comprising:
(i) a polynucleotide comprising a nucleotide sequence encoding a targeting unit that targets antigen-presenting cells, a multimerization unit, such as a dimerization unit, and an antigenic unit, wherein the antigenic unit comprises (A) n−1 antigenic subunits, each subunit comprising a neoepitope and a subunit linker, and (B) a terminal neoepitope, and wherein n is the number of neoepitopes in said antigenic unit and n is an integer of from 1 to 50; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject having cancer, the method comprising administering to the subject:
(a) an anticancer vaccine comprising:
(i) a polynucleotide comprising a nucleotide sequence encoding a targeting unit that targets antigen-presenting cells, a multimerization unit and an antigenic unit, wherein the antigenic unit comprises (A) n−1 antigenic subunits, each subunit comprising a neoepitope and a subunit linker, and (B) a terminal neoepitope, and wherein n is the number of neoepitopes in said antigenic unit and n is an integer of from 1 to 50; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a method for treating a subject having cancer, the method comprising administering to the subject:
(a) an anticancer vaccine comprising:
(i) a polynucleotide comprising a nucleotide sequence encoding a targeting unit that targets antigen-presenting cells, a dimerization unit and an antigenic unit, wherein the antigenic unit comprises (A) n−1 antigenic subunits, each subunit comprising a neoepitope and a subunit linker, and (B) a terminal neoepitope, and wherein n is the number of neoepitopes in said antigenic unit and n is an integer of from 1 to 50; or
(ii) a polypeptide encoded by the polynucleotide as defined in (i); or
(iii) a dimeric protein consisting of two polypeptides as defined in (ii); and
(b) one or more checkpoint inhibitors.

The neoepitope preferably has a length suitable for presentation by HLA (human leukocyte antigen) molecules. Thus, in a preferred embodiment, the neoepitope has a length of from 7 to 30 amino acids. More preferred are neoepitopes having a length of from 7 to 10 amino acids or of from 13 to 30 amino acids, e.g. from 20 to 30 amino acids, e.g. 27 amino acids.

HLA is a major histocompatibility complex (MHC) in humans. There are two primary classes of MHC molecules, MHC class I and MHC II. The terms MHC class I and MHC class II are interchangeably used herein with HLA class I and HLA class II, the terms HLA allele and HLA molecule are also interchangeably used herein.

Preferably, the antigenic unit comprises a plurality of neoepitopes, i.e. several neoepitopes. In one embodiment, the several neoepitopes are different neoepitopes. In another embodiment, the antigenic unit comprises multiple copies of the same neoepitope. In yet another embodiment, the antigenic unit comprises several different neoepitopes and multiple copies of the same neoepitope.

Accordingly, a preferred approach is to include as many neoepitopes as possible in the antigenic unit (i.e. different and/or multiple copies of the same neoepitope) to thereby attack the cancer efficiently without compromising the vaccine's ability to activate T cells against the neoepitopes due to dilution of the desired T cell effect. Further, to secure that all neoepitopes are loaded efficiently to the same antigen-presenting cell, all neoepitope-encoding nucleotide sequences are comprised in a continuous polynucleotide chain resulting in the expression of a protein comprising all the neoepitopes instead of expressing each neoepitope as a discrete peptide.

To design the antigenic unit, the patient's tumor exome is analyzed to identify neoantigens. Preferably, the sequences of the most immunogenic neoepitopes from one or more neoantigens are selected for inclusion into the antigenic unit.

The antigenic unit comprises at least 1 neoepitope, preferably at least 3 neoepitopes, more preferably at least 5 neoepitopes, such as 7 neoepitopes. In a more preferred embodiment, the antigenic unit comprises at least 10 neoepitopes. In another more preferred embodiment, the antigenic unit comprises at least 15 neoepitope sequences, such as at least 20 or at least 25 or at least 30 or at least 35 or at least 40 or at least 45 neoepitope sequences.

### Antigenic Unit of Individualized Anticancer Vaccines Comprising One or More Patient-Present Shared Cancer Antigens or Parts Thereof

Shared tumor antigens are expressed by many tumors, either across patients with the same cancer type, or across patients and cancer types. An example is the HPV16 antigen, a viral antigen that is expressed in about 50% of all patients with squamous cell carcinoma of the head and neck, but also in patients with other cancers such as cervical cancer and vulvar squamous cell carcinoma. Many of these shared antigens have previously been characterized as immunogenic and/or are known, i.e. their immunogenicity has been confirmed by appropriate methods and the results have been published, e.g. in a scientific publication. Others have already been predicted to be presented on specific HLA class I or class II alleles, e.g. by algorithms known in the art and their predicted immunogenicity has been published, e.g. in a scientific publication, without having confirmed their immunogenicity by appropriate methods.

In one embodiment, the antigenic unit comprises one or more patient-present shared cancer antigens or parts thereof, e.g. patient-present shared cancer epitopes, which are known to be immunogenic, have known expression patterns and/or are known or have already been predicted to bind to specific HLA class I and class II alleles.

T cells specific to patient-present shared cancer antigens can travel to the tumor and affect the tumor microenvironment, thus increasing the likelihood that additional tumor-specific T cells are able to attack the cancer.

Thus, in one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit, such as a dimerization unit, and an antigenic unit, wherein the antigenic unit comprises one or more patient-present shared cancer antigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit and an antigenic unit, wherein the antigenic unit comprises one or more patient-present shared cancer antigens or parts thereof; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a dimerization unit and an antigenic unit, wherein the antigenic unit comprises one or more patient-present shared cancer antigens or parts thereof; or
(ii) a polypeptide encoded by the polynucleotide as defined in (i); or
(iii) a dimeric protein consisting of two polypeptides as defined in (ii); and
(b) one or more checkpoint inhibitors.

In one embodiment, the patient-present shared cancer antigen is selected from the group consisting of overexpressed cellular proteins, aberrantly expressed cellular proteins, cancer testis antigens, viral antigens, differentiation antigens, mutated oncogenes and mutated tumor suppressor genes, oncofetal antigens, shared fusion antigens, shared intron retention antigens, dark matter antigens and shared antigens caused by spliceosome mutations or frameshift mutations.

In one embodiment, the patient-present shared cancer antigen is an overexpressed or aberrantly expressed human cellular protein, i.e. a cellular protein found at increased levels in tumors compared with normal, healthy cells and tissues. Examples of such overexpressed or aberrantly expressed cellular proteins include tumor protein D52, Her-2/neu, hTERT (telomerase) and survivin.

In another embodiment, the patient-present shared cancer antigen is a cancer testis antigen which is normally expressed in male germ cells in the testis but not in adult somatic tissues. In some cases, such antigens are also expressed in ovary and trophoblast. In malignancy, this gene regulation is disrupted, resulting in antigen expression in a proportion of tumors of various types. Examples of cancer testis antigens include MAGE-A, MAGE-B, GAGE, PAGE-1, SSX, HOM-MEL-40 (SSX2), NY-ESO-1, LAGE-1 and SCP-1.

In yet another embodiment, the patient-present shared cancer antigen is a differentiation antigen, for example tyrosinase.

In yet another embodiment, the patient-present shared antigen is a viral antigen. Examples of viral antigens include human papilloma virus (HPV), hepatitis B virus (HBV), Epstein-Barr virus (EBV), Kaposi's sarcoma-associated herpesvirus (KSHV), Merkel cell polyomavirus (MCV or MCPyV), human cytomegalovirus (HCMV) and human T-lymphotropic virus (HTLV).

In yet another embodiment, the patient-present shared cancer antigen is a mutated oncogene. Examples of mutated oncogenes include KRAS, CALR and TRP-2.

In yet another embodiment, the patient-present shared cancer antigen is a mutated tumor suppressor gene. Examples include mutated p53, mutated pRB, mutated BCL2 and mutated SWI/SNF.

In yet another embodiment, the patient-present shared cancer antigen is an oncofetal antigen, for example alpha-fetoprotein or carcinoembryonic antigen.

In yet another embodiment, the patient-present shared antigen is a shared intron retention antigen or shared antigen caused by frameshift mutation, for example CDX2 or CALR.

In yet another embodiment, the patient-present shared antigen is a shared antigen caused by spliceosome mutations. An example is an antigen caused by mutations like SF3B1 mut.

Generally, for any cancer antigen, immune tolerance has likely occurred when a patient presents with cancer. An anticancer vaccine should specifically trigger immune response to the antigens incorporated in the vaccine. The peripheral immune tolerance to the selected antigens may be weak or strong. By incorporating such patient-present shared cancer antigens or one or more parts thereof in the antigenic unit—either alone or together with other patient-present shared cancer antigens or parts thereof and/or neoantigens or neoepitopes—a vaccine comprising such antigenic unit elicits an immune response which is strong and broad enough to affect the tumor microenvironment and change the patient's immune response against the tumor from a suppressive/tolerated type to a pro-inflammatory type. This may help to break tolerance to several other antigens, thus representing a considerable clinical benefit for the patient. The afore-described concept may be referred to as tipping the cancer immunity set point.

In one embodiment the antigenic unit comprises one or more patient-present shared cancer antigens or parts thereof that is a human cellular protein, preferably an overexpressed or aberrantly expressed human cellular protein or a differentiation antigen.

The patient-present shared cancer antigen may be detected in the tissue or body fluid of the patient by methods known in the art, including:
sequencing the patient's genome or exome and optionally searching, e.g. by tailor made software, in whole genome/exome-seq data to e.g. identify mutated oncogenes or mutated tumor suppressor genes;
immunohistochemistry of the patient's tumor tissue, e.g. to detect the presence of mutated proteins;
RT-PCR, e.g. to detect the presence of viral antigens or known mutations in oncogenes;
ELISA using antibodies against e.g. mutated tumor proteins in serum samples;
RNA-seq of tumor tissue and comparison to healthy tissue to detect e.g. expression/over-expression of shared cancer antigens;
Searching, e.g. by tailor-made software, in raw RNA sequence data to identify intron retention antigens;
Searching, e.g. by tailor-made software, in whole genome-seq data to identify transposable elements which are elements of dark matter antigens;
detection of short repeats in raw whole exome/RNA sequence data to identify e.g. dark matter antigens;
RNA-seq data to identify e.g. shared viral antigens; and
comparing RNA-seq of the patient's tumor samples with either patient's own healthy tissue or a cohort/database (e.g. TCGA) versus consensus transcript expression, such as GTEX/HPA gene expression data.

In a preferred embodiment, the antigenic unit comprises one or more patient-present shared cancer antigens or part(s) of such antigen(s) that is known to be immunogenic, e.g. has previously been described to elicit an immune response in other patients, or has been predicted to bind to the patient's HLA class I and/or class II alleles.

In another preferred embodiment, the antigenic unit comprises one or more parts of one or more patient-present shared cancer antigens, e.g. one or more epitopes that are known to be immunogenic or have been predicted to bind to the patient's HLA class I and/or class II alleles.

In one embodiment, the antigenic unit comprises one or more patient-present shared cancer epitopes. In a preferred embodiment, such epitopes have a length suitable for presentation by the patient's HLA alleles.

In one embodiment, the antigenic unit comprises one or more patient-present shared cancer epitopes having a length suitable for specific presentation on HLA class I or HLA class II. In one embodiment, the epitope has a length of from 7 to 11 amino acids for HLA class I presentation. In another embodiment, the epitope has a length of from 13 to 30 amino acids for HLA class II presentation.

In one embodiment, the antigenic unit comprises one or more patient-present shared cancer epitopes having a length of from 7 to 30 amino acids, e.g. from 7 to 10 amino acids (such as 7, 8, 9 or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

The antigenic unit may comprise one or more patient-present shared cancer antigens either in full length or one or more parts thereof.

In one embodiment, the antigenic unit comprises one patient-present shared cancer antigen in full length. In another embodiment, the antigenic unit comprises several patient-present shared cancer antigens, each of them in full length.

In yet another embodiment, the antigenic unit comprises one or more parts of a patient-present shared cancer antigen, e.g. one or more patient-present shared cancer epitopes. In yet another embodiment, the antigenic unit comprises a one or more parts of several patient-present shared cancer antigens, e.g. one or more epitopes of several patient-present shared cancer antigens.

In yet another embodiment, the antigenic unit comprises one or more patient-present shared antigens in full length and one or more parts of one or more patient-present shared cancer antigens. Examples include:
antigenic units comprising one patient-present shared antigen in full length and one or more epitopes of one patient-present shared cancer antigen;
antigenic units comprising several patient-present shared cancer antigens, each of them in full length, and one or more epitopes of one patient-present shared cancer antigen;
antigenic units comprising one patient-present shared antigen in full length and one or more epitopes of several patient-present shared cancer antigens; and
antigenic units comprising several patient-present shared cancer antigens, each of them in full length, and one or more epitopes of several patient-present shared cancer antigens.

In a preferred embodiment, the aforementioned epitopes are already known to be immunogenic (e.g. have been described to be immunogenic in the literature) or have already been predicted to bind to the patient's HLA class I and class II alleles (e.g. as described in the literature), preferably have already been predicted to bind to the patient's HLA class I alleles. In another preferred embodiment, the immunogenicity of the aforementioned epitopes is predicted, e.g. the binding of the epitopes to one or more of the patient's HLA class I and/or HLA class II molecules is predicted by methods known in the art, such as those disclosed in WO 2021/205027 A1, the disclosures of which is incorporated herein by reference, or those described in the section “Methods for designing an antigenic unit of an individualized anticancer vaccine for use in the invention” included herein.

In one embodiment, the antigenic unit comprises 1 to 10 patient-present shared antigens in full length.

In another embodiment, the antigenic unit comprises 1 to 30 parts of one or more patient-present shared antigens, wherein these parts include one or multiple epitopes that are predicted to bind to a patient's HLA class I or class II alleles. In yet another embodiment, the antigenic unit comprises 1 to 50 patient-present shared cancer epitopes, optionally epitopes that are predicted to bind to the patient's HLA class I or class II alleles.

### Antigenic Units of Individualized Anticancer Vaccines Comprising One or More Patient-Present Shared Cancer Antigens or Parts Thereof and One or More Neoantigens or Parts Thereof

Such antigenic units are basically a combination of a) all of the afore-described embodiments relating to antigenic units, which comprise one or more patient-present shared cancer antigens or parts thereof and b) all of the afore-described embodiments relating to antigenic units, which comprise one or more neoantigens or parts thereof.

Thus, in one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit, such as a dimerization unit, and an antigenic unit, wherein the antigenic unit comprises one or more patient-present shared cancer antigens or parts thereof and one or more neoantigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit and an antigenic unit, wherein the antigenic unit comprises one or more patient-present shared cancer antigens or parts thereof and one or more neoantigens or parts thereof; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a dimerization unit and an antigenic unit, wherein the antigenic unit comprises one or more patient-present shared cancer antigens or parts thereof and one or more neoantigens or parts thereof; or
(ii) a polypeptide encoded by the polynucleotide as defined in (i); or
(iii) a dimeric protein consisting of two polypeptides as defined in (ii); and
(b) one or more checkpoint inhibitors.

### Methods for Designing an Antigenic Unit of an Individualized Anticancer Vaccine for Use in the Invention

The patient-present shared cancer antigens and neoantigens identified in a particular patient are preferably further processed to find those antigens that will render the vaccine most effective, when included into the antigenic unit. The way and order in which such processing is done depends on how said antigens were identified, i.e. the data that form the basis for such processing.

In one embodiment, the processing and selecting of the antigen(s) to be included in the antigenic unit is carried out as follows:
1) A search in the literature and/or in one or more databases is carried out to retrieve information about and sequences of shared cancer antigens and preferably information about their expression pattern, immunogenicity or predicted immunogenicity, epitopes and/or HLA presentation. Such search is also carried out to determine whether the identified antigen is a patient-present shared cancer antigen or a neoantigen.
2) If it was determined that the identified antigen is a patient-present shared cancer antigen, the sequence thereof is studied to identify epitopes, preferably all epitopes, that are predicted to bind to the patient's HLA class I/II alleles. The prediction may be carried out by using prediction tools known in the art, e.g. prediction software known in the art, such as NetMHCpan and similar software.
3) The most promising sequences of the patient-present shared cancer antigen which are most immunogenic or predicted to be most immunogenic, i.e. those that show predicted binding to one or more of the patient's HLA class I/II alleles, are selected for inclusion into the antigenic unit. In one embodiment, minimal epitopes are selected, e.g. if only a few promising epitopes were identified in step 2 or if longer stretches of non-immunogenic sequences are present between the epitopes. In another embodiment, a longer sequence is selected which comprises several epitopes that bind to the patient's specific HLA alleles. In yet another embodiment, the full-length sequence of the antigen is selected for inclusion into the antigenic unit.
4) The most promising parts of neoantigen sequences, e.g. neoepitopes, are selected for inclusion into the antigenic unit based on predicted immunogenicity and binding to the patient's HLA class I/II alleles of such sequences.

Tumor mutations are discovered by sequencing of tumor and normal tissue and comparing the obtained sequences from the tumor tissue to those of the normal tissue. A variety of methods is available for detecting the presence of a particular mutation or allele in a patient's DNA or RNA. Such methods include dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. Alternatively, mutations may be identified by direct protein sequencing.

Out of the maybe hundreds or thousands of mutations in the tumor exome, the most promising sequences are selected in silico based on predictive HLA-binding algorithms. The intention is to identify all relevant epitopes and after a ranking or scoring, determine the sequences to be included in the antigenic unit. Methods known in the art may be suitable for scoring, ranking and selecting neoepitopes include those disclosed in WO 2020/065023A1 and WO 2020/221/783A1.

Further, any suitable algorithm for such scoring and ranking may be used, including the following:

Available free software analysis of peptide-MHC binding (IEDB and NetMHCpan) that can be downloaded from the following websites:
www.iedb.org
www.cbs.dtu.dk/services/NetMHC

Commercially available advanced software to predict optimal sequences for vaccine design are found e.g. here:
www.oncoimmunity.com/
omictools.com/t-cell-epitopes-category
github.com/griffithlab/pVAC-Seq
crdd.osdd.net/raghava/cancertope/help.php
www.epivax.com/tag/neoantigen/WBD

Each mutation is scored with respect to its antigenicity or immunogenicity, and the most antigenic or immunogenic neoepitopes are selected and optimally arranged in the antigenic unit, e.g. as described herein.

### Antigenic Unit of Non-Individualized Anticancer Vaccines

A non-individualized or “off-the-self” anticancer vaccine (also referred to as shared anticancer vaccine) comprises an antigenic unit which comprises one or more shared cancer antigens or parts thereof.

“Shared cancer antigen” or “shared tumor antigen” is used herein to describe an antigen that has been described to be expressed by many tumors, either across patients with the same cancer type, or across patients and cancer types.

“Shared cancer epitope” is used herein to describe an amino acid sequence comprised in a shared cancer antigen, which is known or has been predicted to be immunogenic.

In one embodiment, the antigenic unit of such non-individualized anticancer vaccines comprises one or more shared cancer antigens or parts thereof, e.g. shared cancer epitopes, which are known to be immunogenic, have known expression patterns and/or are known or have been predicted to bind to specific HLA class I and class II molecules.

Thus, in one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit, such as a dimerization unit, and an antigenic unit, wherein the antigenic unit comprises one or more shared cancer antigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit and an antigenic unit, wherein the antigenic unit comprises one or more shared cancer antigens or parts thereof; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a dimerization unit and an antigenic unit, wherein the antigenic unit comprises one or more shared cancer antigens or parts thereof; or
(ii) a polypeptide encoded by the polynucleotide as defined in (i); or
(iii) a dimeric protein consisting of two polypeptides as defined in (ii); and
(b) one or more checkpoint inhibitors.

Examples of shared cancer antigens include those disclosed under the heading “Antigenic unit of individualized anticancer vaccines comprising one or more patient-present shared cancer antigens or parts thereof” and scFvs derived from a monoclonal Ig produced by myeloma or lymphoma, also called the myeloma/lymphoma M component in patients with B cell lymphoma or multiple myeloma, telomerase, e.g. hTERT, HIV derived sequences like e.g. gpl20 or Gag derived sequences, tyrosinase, tyrosinase related protein (TRP)-1 or TRP-2, melanoma antigen, prostate specific antigen and idiotypes, HPV antigens such as anyone selected from the list consisting of El, E2, E6, E7, LI and L2, e.g. E6 and/or E7 of HPV16 and/or HPV18.

Any shared cancer antigen sequence of sufficient length that includes a specific epitope may be used as the antigenic unit. Accordingly, in one embodiment, the antigenic unit comprises an amino acid sequence of at least 7 amino acids, such as at least 8 amino acids, corresponding to at least about 21 nucleotides, such as at least 24 nucleotides, in a nucleic acid sequence encoding such antigenic unit.

In yet another embodiment, the antigenic unit comprises one or more parts of a shared cancer antigen, e.g. one or more shared cancer epitopes. In yet another embodiment, the antigenic unit comprises one or more parts of several shared cancer antigens, e.g. one or more epitopes of several shared cancer antigens. In yet another embodiment, the antigenic unit comprises one or more shared antigens in full length and one or more parts of one or more shared cancer antigens. Examples include:
antigenic units comprising one shared antigen in full length and one or more epitopes of one shared cancer antigen; and
antigenic units comprising several shared cancer antigens, each of them in full length and one or more epitopes of one shared cancer antigen; and
antigenic units comprising one shared antigen in full length and one or more epitopes of several shared cancer antigens; and
antigenic units comprising several shared cancer antigens, each of them in full length, and one or more epitopes of several shared cancer antigens.

Examples of shared anticancer vaccines comprising shared HPV antigens are disclosed in WO 2013092875A1, which is incorporated herein by reference.

### Methods for Designing an Antigenic Unit of a Shared Anticancer Vaccine for Use in the Method of the Invention

Also for shared anticancer vaccines for use in the method of the invention, the antigenic unit is designed to include those sequences that are likely to render the vaccine effective in a variety of patients, e.g. patients having a certain type of cancer.

In one embodiment, the selection of the antigen to be included in the antigenic unit is carried out by performing a search in the literature and/or in one or more databases to retrieve information about and sequences of shared cancer antigens and preferably information about their expression pattern, immunogenicity or predicted immunogenicity, epitopes and/or HLA presentation. Epitopes are then identified that are known or predicted to bind to a variety of HLA class I/II alleles of many patients or that bind a certain subset of HLA class I/II alleles which is dominant in a certain cancer indication and/or a certain patient population across different cancer indications. Preferably, the most promising, i.e. the sequences of the shared cancer antigen which are most immunogenic or predicted to be most immunogenic are selected for inclusion into the antigenic unit.

### Further Embodiments of the Antigenic Unit

The following applies to the antigenic unit in the anticancer vaccines for use in the method of the invention in general, i.e. to the antigenic unit comprised in individualized anticancer vaccines and to the antigenic unit comprised in shared anticancer vaccines.

The term antigen is used in this section of the application for a neoantigen, a neoepitope, a patient-present shared cancer antigen, a part of a patient-present shared cancer antigen, such as a patient-present shared cancer epitope, a shared cancer antigen, and a part of a shared cancer antigen, such as a shared cancer epitope.

In one embodiment, the antigenic unit comprises only one copy of each antigen, so that when e.g. 10 different antigens are comprised in the antigenic unit, a vaccine comprising said antigenic unit may elicit a cell-mediated immune response against all 10 different antigens and thus attack the cancer efficiently.

However, if only a few antigens are included in the antigenic unit, then the antigenic unit may comprise at least two copies of a particular antigen in order to strengthen the immune response to the antigen.

Such a situation may arise in the design of an antigenic unit for an individualized anticancer vaccine, when only a few neoantigens/neoepitopes could be identified in a patient that are predicted to be sufficiently immunogenic/predicted to bind to the patient's HLA alleles. If in such patient patient-present shared cancer antigens are identified in addition to neoantigens/neoepitopes, it is preferred to then include such one or more patient-present shared cancer antigen sequences or parts thereof into the antigenic unit rather than including multiple copies of the same neoantigen/neoepitope.

The length of the antigenic unit is determined by the length of the antigen(s) comprised therein as well as their number. In one embodiment, the antigenic unit comprises from about 21 to about 2000 amino acids, preferably from about 30 amino acids to about a 1500 amino acids, more preferably from about 50 to about 1000 amino acids, such as from about 100 to about 500 amino acids or from about 100 to about 400 amino acids or from about 100 to about 300 amino acids.

In another embodiment, the antigenic unit comprises up to 3500 amino acids, such as from 60 to 3500 amino acids, e.g. from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

In order to enhance the immune response, for an individualized anticancer vaccine, the antigens may be arranged in the antigenic subunit as described in the following: the antigenic unit can be described as a polypeptide having an N-terminal start and a C-terminal end. The antigenic unit is connected to the dimerization unit, preferably via a unit linker. The antigenic unit is either located at the COOH-terminal end or the NH2-terminal end of the polypeptide/dimeric protein. It is preferred that the antigenic unit is in the COOH-terminal end of the polypeptide/dimeric protein. In one embodiment, the antigens, preferably epitopes, are arranged in the order of more antigenic to less antigenic in the direction from the N-terminal start of the antigenic unit to the C-terminal end of the antigenic unit. Alternatively, particularly if the hydrophilicity/hydrophobicity varies greatly among the antigens, it is preferred that the most hydrophobic antigens is/are substantially positioned in the middle of the antigenic unit and the most hydrophilic antigens is/are positioned at the N-terminal start and/or the C-terminal end of the antigenic unit.

Since a true positioning in the middle of the antigenic unit is only possible if the antigenic unit comprises an odd number of antigens, the term “substantially” in this context refers to antigenic units comprising an even number of antigens, wherein the most hydrophobic antigens are positioned as closed to the middle as possible.

By way of example, an antigenic unit comprises 5 antigenic subunits, each comprising a different epitope, e.g. a different neoepitope, which are arranged as follows: 1-2-3*-4-5; with 1, 2, 3*, 4 and 5 each being a different neoepitope and—being a subunit linker and * indicating the most hydrophobic neoepitope, which is positioned in the middle of the antigenic unit.

In another example, an antigenic unit comprises 6 antigenic subunits, each comprising a different epitope, e.g. a different neoepitope, which are arranged as follows: 1-2-3*-4-5-6 or, alternatively, as follows: 1-2-4-3*-5-6; with 1, 2, 3*, 4, 5 and 6 each being a different neoepitope and—being a subunit linker and * indicating the most hydrophobic neoepitope, which is positioned substantially in the middle of the antigenic unit.

Alternatively, the antigenic subunits may be arranged such that they alternate between a hydrophilic and a hydrophobic antigen.

Optionally, GC rich sequences encoding antigens (e.g. GC rich sequences encoding neoepitopes or epitopes) are arranged in such a way, that GC clusters are avoided. In one embodiment, GC rich sequences encoding for antigens are arranged such that there is at least one non-GC rich sequence between them.

In one embodiment, the antigenic unit comprises one or more linkers. In another embodiment, the antigenic unit comprises multiple antigens, e.g. multiple epitopes, e.g. neoepitopes, wherein the antigens are separated by linkers. In yet another embodiment, the antigenic unit comprises multiple antigens wherein each antigen is separated from other antigens by linkers. An alternative way to describe the separation of each antigen from other antigens by linkers is that all but the terminal antigen, i.e. the antigen at the N-terminal start of the polypeptide or the C-terminal end of the polypeptide (i.e. located at the end of the antigenic unit that is not connected to the dimerization unit), are arranged in antigenic subunits, wherein each subunit comprises or consists of an antigen e.g. a neoepitope, and a subunit linker.

Hence, an antigenic unit comprising n antigens comprises n−1 antigenic subunits, wherein each subunit comprises an antigen and a subunit linker, and further comprises a terminal antigen. In one embodiment, wherein n is an integer of from 1 to 50, e.g. 3 to 50 or 15 to 40 or 10 to 30 or 10 to 25 or 10 to 20 or 15 to 30 or 15 to 25 or 15 to 20.

Due to the separation of the antigens by the linkers, each antigen is presented in an optimal way to the immune system.

Thus, in one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit, such as dimerization unit, and an antigenic unit, wherein the antigenic unit comprises (A) n−1 antigenic subunits, each subunit comprising a subunit linker and an antigen selected from the group consisting of a neoantigen, a neoepitope, a patient-present shared cancer antigen, a part of a patient-present shared cancer antigen and a patient-present shared cancer epitope, or selected from the group consisting of a shared cancer antigen, a part of a shared cancer antigen and a shared cancer epitope and (B) a terminal antigen, and wherein n the number of antigens in said antigenic unit and n is an integer of from 1 to 50; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit and an antigenic unit, wherein the antigenic unit comprises (A) n−1 antigenic subunits, each subunit comprising a subunit linker and an antigen selected from the group consisting of a neoantigen, a neoepitope, a patient-present shared cancer antigen, a part of a patient-present shared cancer antigen and a patient-present shared cancer epitope or selected from the group consisting of a shared cancer antigen, a part of a shared cancer antigen and a shared cancer epitope, and (B) a terminal antigen, and wherein n the number of antigens in said antigenic unit and n is an integer of from 1 to 50; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a method for treating a subject having cancer, the method comprising administering to the subject
(a) an anticancer vaccine comprising
(i) a polynucleotide comprising a nucleotide sequence encoding a targeting unit that targets antigen-presenting cells, a dimerization unit and an antigenic unit, wherein the antigenic unit comprises (A) n−1 antigenic subunits, each subunit comprising a subunit linker and an antigen selected from the group consisting of a neoantigen, a neoepitope, a patient-present shared cancer antigen, a part of a patient-present shared cancer antigen and a patient-present shared cancer epitope or selected from the group consisting of a shared cancer antigen, a part of a shared cancer antigen and a shared cancer epitope, and (B) a terminal antigen, and wherein n the number of antigens in said antigenic unit and n is an integer of from 1 to 50; or
(ii) a polypeptide encoded by the polynucleotide as defined in (i); or
(iii) a dimeric protein consisting of two polypeptides as defined in (ii); and
(b) one or more checkpoint inhibitors.

### Linkers in the Antigenic Unit

Linkers in the antigenic unit separate antigens comprised therein, e.g. epitopes. As described above, all antigens, e.g. neoepitopes, may be separated from each other by linkers and arranged in subunits. In the following, the term subunit linker and linker are used interchangeably, and both denote a linker in the antigenic unit.

In one embodiment, the subunit linker is designed to be non-immunogenic. It may be a rigid linker, meaning that that it does not allow the two amino acid sequences that it connects to substantially move freely relative to each other. Alternatively, it may be a flexible linker, i.e. a linker that allows the two amino acid sequences that it connects to substantially move freely relative to each other.

Both types of linkers are useful. In one embodiment, the subunit linker is a flexible linker, which allows for presenting the antigen in an optimal manner to the T cells, even if the antigenic unit comprises a large number of antigens.

In one embodiment, the subunit linker is a peptide consisting of from 4 to 40 amino acids, e.g. 35, 30, 25 or 20 amino acids, e.g. from 5 to 20 amino acids or 5 to 15 amino acids or 8 to 20 amino acids or 8 to 15 amino acids 10 to 15 amino acids or 8 to 12 amino acids. In another embodiment, the subunit linker consists of 10 amino acids.

In one embodiment, e.g. in an antigenic unit comprising neoepitopes, the subunit linker is identical in all antigenic subunits. If, however, one or more of the antigens comprise a sequence similar to that of the linker, it may be an advantage to substitute the neighboring subunit linkers with a linker of a different sequence. Also, if an antigen-subunit linker junction is predicted to constitute an immunogenic epitope in itself, then a linker of a different sequence might be used.

In one embodiment, the subunit linker is a flexible linker, preferably a flexible linker which comprises small, non-polar (e.g. glycine, alanine or leucine) or polar (e.g. serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens. In one embodiment, the flexible linker is a serine (S) and/or glycine (G) rich linker, i.e. a linker comprising several serine and/or several glycine residues. Preferred examples are GGGGS (SEQ ID NO: 24), GGGSS (SEQ ID NO: 25), GGGSG (SEQ ID NO: 26), GGSGG (SEQ ID NO: 27), SGSSGS (SEQ ID NO: 28) or multiple variants thereof such as GGGGSGGGGS (SEQ ID NO: 29), (GGGGS)m (SEQ ID NO: 30), (GGGSS)m (SEQ ID NO: 31), (GGSGG)m (SEQ ID NO: 32), (GGGSG)m (SEQ ID NO: 33) or (SGSSGS)m (SEQ ID NO: 34), where m is an integer from 1 to 5, e.g., 1, 2, 3, 4, or 5. In a preferred embodiment, m is 2. In another preferred embodiment, the serine and/or glycine rich linker further comprises at least one leucine (L) residue, such as at least 1 or at least 2 or at least 3 leucine residues, e.g. 1, 2, 3 or 4 leucine residues.

In one embodiment, the subunit linker comprises or consists of LGGGS (SEQ ID NO: 35), GLGGS (SEQ ID NO: 36), GGLGS (SEQ ID NO: 37), GGGLS (SEQ ID NO: 38) or GGGGL (SEQ ID NO: 39). In another embodiment, the subunit linker comprises or consists of LGGSG (SEQ ID NO: 40), GLGSG (SEQ ID NO: 41), GGLSG (SEQ ID NO: 42), GGGLG (SEQ ID NO: 43) or GGGSL (SEQ ID NO: 44). In yet another embodiment, the subunit linker comprises or consists of LGGSS (SEQ ID NO: 45), GLGSS (SEQ ID NO: 46) or GGLSS (SEQ ID NO: 47).

In yet another embodiment, the subunit linker comprises or consists of LGLGS (SEQ ID NO: 48), GLGLS (SEQ ID NO: 49), GLLGS (SEQ ID NO: 50), LGGLS (SEQ ID NO: 51) or GLGGL (SEQ ID NO: 52). In yet another embodiment, the subunit linker comprises or consists of LGLSG (SEQ ID NO: 53), GLLSG (SEQ ID NO: 54), GGLSL (SEQ ID NO: 55), GGLLG (SEQ ID NO: 56) or GLGSL (SEQ ID NO: 57). In yet another embodiment, the subunit linker comprises or consists of LGLSS (SEQ ID NO: 58), or GGLLS (SEQ ID NO: 59).

In another embodiment, the subunit linker is serine-glycine linker that has a length of 10 amino acids and comprises 1 or 2 leucine residues.

In one embodiment, the subunit linker comprises or consists of LGGGSGGGGS (SEQ ID NO: 60), GLGGSGGGGS (SEQ ID NO: 61), GGLGSGGGGS (SEQ ID NO: 62), GGGLSGGGGS (SEQ ID NO: 63) or GGGGLGGGGS (SEQ ID NO: 64). In another embodiment, the subunit linker comprises or consists of LGGSGGGGSG (SEQ ID NO: 65), GLGSGGGGSG (SEQ ID NO: 66), GGLSGGGGSG (SEQ ID NO: 67), GGGLGGGGSG (SEQ ID NO: 68) or GGGSLGGGSG (SEQ ID NO: 69). In yet another embodiment, the subunit linker comprises or consists of LGGSSGGGSS (SEQ ID NO: 70), GLGSSGGGSS (SEQ ID NO: 71), GGLSSGGGSS (SEQ ID NO: 72), GGGLSGGGSS (SEQ ID NO: 73) or GGGSLGGGSS (SEQ ID NO: 74).

In a further embodiment, the subunit linker comprises or consists of LGGGSLGGGS (SEQ ID NO: 75), GLGGSGLGGS (SEQ ID NO: 76), GGLGSGGLGS (SEQ ID NO: 77), GGGLSGGGLS (SEQ ID NO: 78) or GGGGLGGGGL (SEQ ID NO: 79). In another embodiment, the subunit linker comprises or consists of LGGSGLGGSG (SEQ ID NO: 80), GLGSGGLGSG (SEQ ID NO: 81), GGLSGGGLSG (SEQ ID NO: 82), GGGLGGGGLG (SEQ ID NO: 83) or GGGSLGGGSL (SEQ ID NO: 84). In yet another embodiment, the subunit linker comprises or consists of LGGSSLGGSS (SEQ ID NO: 85), GLGSSGLGSS (SEQ ID NO: 86) or GGLSSGGLSS (SEQ ID NO: 87).

In yet another embodiment, the subunit linker comprises or consists of GSGGGA (SEQ ID NO: 88), GSGGGAGSGGGA (SEQ ID NO: 89), GSGGGAGSGGGAGSGGGA (SEQ ID NO: 90), GSGGGAGSGGGAGSGGGAGSGGGA SEQ ID NO: 91) or GENLYFQSGG (SEQ ID NO: 92). In yet another embodiment, the subunit linker comprises or consists of SGGGSSGGGS (SEQ ID NO: 93), SSGGGSSGGG (SEQ ID NO: 94), GGSGGGGSGG (SEQ ID NO: 95), GSGSGSGSGS (SEQID NO: 96), GGGSSGGGSG (amino acids 121-130 of SEQ ID NO: 1), GGGSSS (SEQ ID NO: 97), GGGSSGGGSSGGGSS (SEQ ID NO: 98) or GLGGLAAA (SEQ ID NO: 99).

In another embodiment, the subunit linker is a rigid linker. Such rigid linkers may be useful to efficiently separate (larger) antigens and prevent their interferences with each other. In one embodiment, the subunit linker comprises or consist of KPEPKPAPAPKP (SEQ ID NO: 100), AEAAAKEAAAKA (SEQ ID NO: 101), (EAAAK)m (SEQ ID NO: 102), PSRLEEELRRRLTEP (SEQ ID NO: 103) or SACYCELS (SEQ ID NO: 104).

In yet another embodiment, the subunit linker comprises or consists of TQKSLSLSPGKGLGGL (SEQ ID NO: 105). In yet another embodiment, the subunit linker comprises or consists of SLSLSPGKGLGGL (SEQ ID NO: 106).

In yet another embodiment, the subunit linker comprises or consists of GGSAGGSGSGSSGGSSGASGTGTAGGTGSGSGTGSG (SEQ ID NO: 107); or GGSGGGSEGGGSEGGGSEGGGSEGGGSEGGGSGGGS (SEQ ID NO: 108) or ELKTPLGDTTHT (amino acids 94-105 of SEQ ID NO: 1) or EPKSCDTPPPCPRCP (amino acids 106-120 of SEQ ID NO: 1).

In yet another embodiment, the subunit linker is a cleavable linker, e.g. a linker which includes one or more recognition sites for endopeptidases, e.g. endopeptidases such as furin, caspases, cathepsins and the like. Cleavable linkers may be introduced to release free functional protein domains (e.g. encoded by larger antigens), which may overcome steric hindrance between such domains or other drawbacks due to interference of such domains, like decreased bioactivity, altered biodistribution.

Examples of further sequences encoded by subunit linkers are disclosed in paragraphs [0098]-[0099] and in the recited sequences of WO 2020/176797A1, which is incorporated herein by reference, and in paragraphs to of US 2019/0022202A1, which is incorporated herein by reference.

### Unit Linker

The antigenic unit is connected to the dimerization unit, preferably by a unit linker. Thus, in one embodiment, the anticancer vaccine for use in the method of the invention comprises a nucleotide sequence encoding a unit linker that connects the antigenic unit to the dimerization unit.

The unit linker may comprise a restriction site in order to facilitate the construction of the polynucleotide. In one embodiment, the unit linker is GLGGL (SEQ ID NO: 52) (nucleotide sequence with SEQ ID NO: 10) or GLSGL (SEQ ID NO: 109). In another embodiment, the unit linker comprises or consists of GGGGS (SEQ ID NO: 24), GGGGSGGGGS (SEQ ID NO: 29), (GGGGS)m (SEQ ID NO: 30), EAAAK (SEQ ID NO: 110), (EAAAK)m (SEQ ID NO: 102), (EAAKG)mS (SEQ ID NO: 111), GPSRLEEELRRRLTEPG (SEQ ID NO: 112), AAY or HEYGAEALERAG (SEQ ID NO: 114).

### Targeting Unit

The anticancer vaccine for use in the method of the invention comprises a targeting unit that targets antigen-presenting cells (APCs). APCs include dendritic cells (DCs) and subsets thereof.

The term “targeting unit” as used herein refers to a unit that delivers the polypeptide/dimeric protein (encoded by the polynucleotide as defined above) to an antigen-presenting cell for MHC class Il-restricted presentation to CD4+ T cells or for providing cross presentation to CD8+ T cells by MHC class I restriction.

Due to the targeting unit, the anticancer vaccine for use in the method of the invention attracts DCs, neutrophils and other immune cells. Thus, the anticancer vaccine will not only target the antigenic unit comprised therein to specific cells, but also facilitate a response-amplifying effect (adjuvant effect) by recruiting specific immune cells to the administration site of the anticancer vaccine. This unique mechanism is of great importance in a clinical setting: the anticancer vaccine for use in the method of the invention can be administered to the patient without any adjuvants, since the vaccine itself provides the adjuvant effect.

The targeting unit is designed to target the anticancer vaccine for use in the method of the invention to surface molecules expressed on the APCs, such as molecules expressed exclusively on subsets of DCs.

Examples of such surface molecules on APCs are HLA, cluster of differentiation 14 (CD14), cluster of differentiation 40 (CD40), CLEC9A, chemokine receptors and Toll-like receptors (TLRs). Chemokine receptors include C-C motif chemokine receptor 1 (CCR1), C-C motif chemokine receptor 3 (CCR3), C-C motif chemokine receptor 4 (CCR4), C-C motif chemokine receptor 5 (CCR5), C-C motif chemokine receptor 6 (CCR6), C-C motif chemokine receptor 7 (CCR7), C-C motif chemokine receptor 8 (CCR8) and XCR1. Toll-like receptors include TLR-2, TLR-4 and TLR-5. The targeting unit is or comprises a moiety that interacts with these surface molecules.

Thus, in one embodiment, the targeting unit comprises or consist of an antibody-binding region, such as the antibody variable domains (VL and VH), with specificity for HLA, CD14, CD40, CLEC9A or Toll-like receptors. In another embodiment, the targeting unit comprises or consists of a synthetic or natural ligand. Examples include soluble CD40 ligand, natural ligands like chemokines, such as in their human forms, e.g. chemokine ligand 5, also called C-C motif ligand 5 (CCL5 or RANTES), macrophage inflammatory protein alpha and its isoforms, including mouse CCL3 (or MIP-1a), and human isoforms hCCL3, hCCL3L1, hCCL3L2 and hCCL3L3, chemokine ligand 4 (CCL4) and its isoform CCL4L, chemokine ligand 19 (CCL19), chemokine ligand 20 (CCL20), chemokine ligand 21 (CCL21), chemokine motif ligand 1 or 2 (XCL1 or XCL2) and bacterial antigens like for example flagellin.

In one embodiment, the targeting unit has affinity for an MHC class II protein. Thus, in one embodiment, the targeting unit comprises or consists of an antibody-binding region, such as the antibody variable domains (VL and VH), with specificity for MHC class II proteins, such as those selected from the group consisting of anti-HLA-DP, anti-HLA-DR and anti-pan HLA class II.

In another embodiment, the targeting unit has affinity for a surface molecule selected from the group consisting of CD14, CD40, TLR-2, TLR-4 and TLR-5. Thus, in one embodiment the targeting unit comprises or consist of an antibody-binding region, such as the antibody variable domains (VL and VH), with specificity for CD14, CD40, TLR-2, TLR4 or TLR-5, such as anti-CD14, anti-CD40, anti-TLR-2, anti-TLR-4 or anti-TLR-5. In yet another embodiment, the targeting unit comprises or consists of flagellin, which has affinity for TLR-5. In yet another embodiment, the targeting unit comprises or consists of an antibody-binding region with specificity for CLEC9A, such as anti-CLEC9A or variants thereof, such as anti-CLEC9A Fv or the targeting unit comprises or consists of a CLEC9 ligand, e.g. a CLEC9 ligand comprising or consisting of the nucleic acid sequence with SEQ ID NO: 115 or an amino acid sequence encoded by said nucleic acid sequence.

Preferably, the targeting unit has affinity for a chemokine receptor selected from CCR1, CCR3, CCR5 and CCR7, more preferably for a chemokine receptor selected from CCR1, CCR3 and CCR5. In one embodiment, the targeting unit has affinity for the chemokine receptor CCR7. In another embodiment, the targeting unit comprises or consists of CCL19, such as CCL19 comprising or consisting of a nucleotide sequence of SEQ ID NO: 121 or an amino acid sequence encoded by said nucleotide sequence, or CCL21, such as the human forms of CCL19 or CCL21.

Preferably, the targeting unit comprises or consists of chemokine human macrophage inflammatory protein alpha (human MIP-1α (hMIP-1α) variant, also called LD78β or CCL3L1), which binds to its cognate receptors, including CCR1 and CCR5 expressed on the cell surface of APCs. The binding of the targeting unit to its cognate receptors leads to internalization of the anticancer vaccine into the APC and degradation of the protein into small peptides that are loaded onto MHC molecules and presented to CD4+ and CD8+ T cells to induce cancer specific immune responses. Once stimulated, and with help from activated CD4+ T cells, CD8+ T cells will target and kill cancer cells expressing the same antigens.

In one preferred embodiment, the targeting unit comprises an amino acid sequence having at least 80% sequence identity to the amino acid sequence 24-93 of SEQ ID NO: 1, such as comprising the amino acid sequence 26-93 of SEQ ID NO: 1 or comprising the amino acid sequence 28-93 of SEQ ID NO: 1.

In a further preferred embodiment, the targeting unit comprises an amino acid sequence having at least 85% sequence identity to the amino acid sequence 24-93 of SEQ ID NO: 1, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence 24-93 of SEQ ID NO: 1.

In a more preferred embodiment, the targeting unit consists of an amino acid sequence having at least 80% sequence identity to the amino acid sequence 24-93 of SEQ ID NO: 1, such as consisting of the amino acid sequence 26-93 of SEQ ID NO: 1 or consisting of the amino acid sequence 28-93 of SEQ ID NO: 1.

In a further preferred embodiment, the targeting unit consists of an amino acid sequence having at least 85% sequence identity to the amino acid sequence 24-93 of SEQ ID NO: 1, such as at least 86% or at least 87%, 88%, 89%, 90%, 91%, 92%, 93%, 94%, 95%, 96%, 97%, 98% or at least 99% sequence identity. In yet another preferred embodiment, the targeting unit consists of the amino acid sequence 24-93 of SEQ ID NO: 1.

In one preferred embodiment, the targeting unit comprises the amino acid sequence 24-93 of SEQ ID NO: 1, except that at the most six amino acids have been substituted, deleted or inserted, such as at the most five amino acids, such as at the most four amino acids, such as at the most three amino acids, such as at the most two amino acids or such as at the most one amino acid. An embodiment of such a targeting unit is one comprising the amino acid sequence 26-93 of SEQ ID NO: 1 or one comprising the amino acid sequence 28-93 of SEQ ID NO: 1.

In another preferred embodiment, the targeting unit consists of the amino acid sequence 24-93 of SEQ ID NO: 1, except that at the most six amino acids have been substituted, deleted or inserted, such as at the most five amino acids, such as at the most four amino acids, such as at the most three amino acids, such as at the most two amino acids or such as at the most one amino acid. An embodiment of such a targeting unit is one consisting of the amino acid sequence 26-93 of SEQ ID NO: 1 or one consisting of the amino acid sequence 28-93 of SEQ ID NO: 1.

In one preferred embodiment, the targeting unit comprises a nucleic acid sequence having at least 80% sequence identity to the nucleic acid sequence with SEQ ID NO: 5.

In a further preferred embodiment, the targeting unit comprises a nucleic acid sequence having at least 85% sequence identity to the nucleic acid sequence with SEQ ID NO: 5, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the nucleic acid sequence of SEQ ID NO: 5.

In a more preferred embodiment, the targeting unit consists of a nucleic acid sequence having at least 80% sequence identity to the nucleic acid sequence with SEQ ID NO: 5.

In a further preferred embodiment, the targeting unit consists of a nucleic acid sequence having at least 85% sequence identity to the nucleic acid sequence of SEQ ID NO: 5, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the nucleic acid sequence of SEQ ID NO: 5.

### Multimerization Unit/Dimerization Unit

The anticancer vaccine for use in the method of the invention comprises a multimerization unit, such as a dimerization unit.

The term “multimerization unit” as used herein, refers to a sequence of nucleotides or amino acids between the antigenic unit and the targeting unit. In addition to connecting the antigenic unit and the targeting unit, the multimerization unit facilitates multimerization of/joins multiple polypeptides, such as two, three, four or more polypeptides, into a multimeric protein, such as a dimeric protein, a trimeric protein or a tetrameric protein. Furthermore, the multimerization unit also provides the flexibility in the multimeric protein to allow optimal binding of the targeting unit to the surface molecules on the APCs, even if they are located at variable distances. The multimerization unit may be any unit that fulfils one or more of these requirements.

Multimerization Unit that Facilitates Multimerization of/Joins More than Two Polypeptides

In one embodiment, the multimerization unit is a trimerization unit, such as a collagen-derived trimerization unit, such as a human collagen-derived trimerization domain, such as human collagen derived XVIII trimerization domain (see for instance A. Alvarez-Cienfuegos et al., Sci Rep 6, 28643 (2016)) or human collagen XV trimerization domain. Thus, in one embodiment, the multimerization unit is a trimerization unit that comprises or consists of the nucleic acid sequence with SEQ ID NO: 116, or an amino acid sequence encoded by said nucleic acid sequence. In another embodiment, the trimerization unit is the C-terminal domain of T4 fibritin. Thus, in one embodiment, the multimerization unit is a trimerization unit that comprises or consists of the amino acid sequence with SEQ ID NO: 117.

In another embodiment, the multimerization unit is a tetramerization unit, such as a domain derived from p53, optionally further comprising a hinge region as described below. Thus, in one embodiment, the multimerization unit is a tetramerization unit that comprises or consists of the nucleic acid sequence with SEQ ID NO: 113, or an amino acid sequence encoded by said nucleic acid sequence, optionally further comprising a hinge region as described below.

### Dimerization Unit

The term “dimerization unit” as used herein, refers to a sequence of nucleotides or amino acids between the antigenic unit and the targeting unit. In addition to connecting the antigenic unit and the targeting unit, the dimerization unit facilitates dimerization of/joins two monomeric polypeptides into a dimeric protein. Furthermore, the dimerization unit also provides the flexibility in the dimeric protein to allow optimal binding of the targeting unit to the surface molecules on the APCs, even if they are located at variable distances. The dimerization unit may be any unit that fulfils one or more of these requirements.

Accordingly, in one embodiment the anticancer vaccine for use in the method of the invention comprises a dimerization unit comprising a hinge region. In another embodiment, the dimerization unit comprises a hinge region and another domain that facilitates dimerization. In yet another embodiment, the dimerization unit comprises a hinge region, a dimerization unit linker and another domain that facilitates dimerization, wherein the dimerization unit linker is located between the hinge region and the other domain that facilitates dimerization. In one embodiment, the dimerization unit linker is a glycine-serine rich linker, preferably GGGSSGGGSG (SEQ ID NO: 118), i.e. the dimerization unit comprises a glycine-serine rich dimerization unit linker and preferably the dimerization unit linker GGGSSGGGSG (SEQ ID NO: 118).

The term “hinge region” refers to an amino acid sequence comprised in the dimerization unit that contributes to joining two of the polypeptides, i.e. contributes to the formation of a dimeric protein. In the context of a multimerization unit that facilitates multimerization of/joins more than two polypeptides, the term “hinge region” refers to an amino acid sequence comprised in such multimerization unit that contributes to joining more than two polypeptides, e.g. three or four polypeptides and/or functioning as a flexible spacer, allowing the targeting units of the multimeric protein to bind simultaneously to multiple surface molecules on APCs, even if these surface molecules are located at variable distances.

Moreover, the hinge region functions as a flexible spacer, allowing the two targeting units of the dimeric protein to bind simultaneously to two surface molecules on APCs, even if they are located at variable distances. The hinge region may be Ig derived, such as derived from IgG, e.g. IgG2 or IgG3. In one embodiment, the hinge region is derived from IgM, e.g. comprising or consisting of the nucleotide sequence with SEQ ID NO: 119 or an amino acid sequence encoded by said nucleic acid sequence. The hinge region may contribute to the dimerization through the formation of covalent bond(s), e.g. disulfide bridge(s) between cysteine residues. Thus, in one embodiment, the hinge region has the ability to form one or more covalent bonds. Preferably, the covalent bond is a disulfide bridge.

In one embodiment, the dimerization unit comprises or consists of a hinge exon hl and hinge exon h4 (human hinge region 1 and human hinge region 4) having an amino acid sequence having at least 80% sequence identity to the amino acid sequence 94-120 of SEQ ID NO: 1.

In a preferred embodiment, the dimerization unit comprises or consists of a hinge exon h1 and hinge exon h4 with an amino acid sequence having at least 85% sequence identity to the amino acid sequence 94-120 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a hinge exon hl and hinge exon h4 with the amino acid sequence 94-120 of SEQ ID NO: 1.

In one preferred embodiment, the dimerization unit comprises or consists of the amino acid sequence 94-120 of SEQ ID NO: 1, except that at the most four amino acids have been substituted, deleted or inserted, such as at the most three amino acids, such as at the most two amino acids or such as at the most one amino acid.

In one preferred embodiment, the dimerization unit comprises or consists of a nucleic acid sequence having at least 80% sequence identity to the nucleic acid sequence with SEQ ID NO: 8.

In a further preferred embodiment, the dimerization unit comprises or consists of a nucleic acid sequence having at least 85% sequence identity to the nucleic acid sequence with SEQ ID NO: 8, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of the nucleic acid sequence of SEQ ID NO: 8.

In another embodiment, the dimerization unit comprises another domain that facilitates dimerization; preferably, said other domain is an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof. Preferably, the other domain that facilitates dimerization is a carboxyterminal C domain derived from IgG. More preferably, the other domain that facilitates dimerization is a carboxyterminal C domain derived from IgG3.

In one embodiment, the dimerization unit comprises or consists of a carboxyterminal C domain derived from IgG3 with an amino acid sequence having at least 80% sequence identity to the amino acid sequence 131-237 of SEQ ID NO: 1.

In a preferred embodiment, the dimerization unit comprises or consists of a carboxyterminal C domain derived from IgG3 with an amino acid sequence having at least 85% sequence identity to the amino acid sequence 131-237 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a carboxyterminal C domain derived from IgG3 with the amino acid sequence 131-237 of SEQ ID NO: 1.

In one preferred embodiment, the dimerization unit comprises or consists of the amino acid sequence 131-237 of SEQ ID NO: 1, except that at the most 16 amino acids have been substituted, deleted or inserted, such as at the most 15, 14, 13, 12, 11, 10, 9, 8, 7, 6, 5, 4, 3, 2, or 1 amino acid.

In one preferred embodiment, the dimerization unit comprises or consists of a nucleic acid sequence having at least 80% sequence identity to the nucleic acid sequence with SEQ ID NO: 10.

In a further preferred embodiment, the dimerization unit comprises or consists of a nucleic acid sequence having at least 85% sequence identity to the nucleic acid sequence with SEQ ID NO: 10, such as at least 86% or at least 87%, 88%, 89%, 90%, 91%, 92%, 93%, 94%, 95%, 96%, 97%, 98% or at least 99% sequence identity. In yet a further preferred embodiment, the dimerization unit comprises or consists of the nucleic acid sequence of SEQ ID NO: 10.

The immunoglobulin domain contributes to dimerization through non-covalent interactions, e.g. hydrophobic interactions. Thus, in one embodiment, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In a preferred embodiment, the dimerization unit comprises a hinge exon h1, a hinge exon h4, a dimerization unit linker and a CH3 domain of human IgG3. In a further preferred embodiment, the dimerization unit comprises a polypeptide consisting of hinge exon h1, hinge exon h4, a dimerization unit linker and a CH3 domain of human IgG3.

In another preferred embodiment, the dimerization unit consists of a polypeptide consisting of hinge exon h1, hinge exon h4, a dimerization unit linker and a CH3 domain of human IgG3.

In one embodiment, the dimerization unit comprises an amino acid sequence having at least 80% sequence identity to the amino acid sequence 94-237 of SEQ ID NO: 1.

In a preferred embodiment, the dimerization unit comprises an amino acid sequence having at least 85% sequence identity to the amino acid sequence 94-237 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a more preferred embodiment the dimerization unit consists of an amino acid sequence having at least 80% sequence identity to the amino acid sequence 94-237 of SEQ ID NO: 1, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In an even more preferred embodiment, the dimerization unit consists of the amino acid sequence 94-237 of SEQ ID NO: 1.

In one preferred embodiment, the dimerization unit comprises the amino acid sequence 94-237 of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In one preferred embodiment, the dimerization unit consists of the amino acid sequence 94-237 of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In one preferred embodiment, the dimerization unit comprises a nucleic acid sequence having at least 80% sequence identity to the nucleic acid sequence with SEQ ID NO: 12.

In a further preferred embodiment, the dimerization unit comprises a nucleic acid sequence having at least 85% sequence identity to the nucleic acid sequence with SEQ ID NO: 12, such as at least 86% or at least 87%, 88%, 89%, 90%, 91%, 92%, 93%, 94%, 95%, 96%, 97%, 98% or at least 99% sequence identity. In yet a further preferred embodiment, the dimerization unit comprises the nucleic acid sequence of SEQ ID NO: 12.

In another preferred embodiment, the dimerization unit consists of a nucleic acid sequence having at least 80% sequence identity to the nucleic acid sequence with SEQ ID NO: 12.

In a further preferred embodiment, the dimerization unit consists of a nucleic acid sequence having at least 85% sequence identity to the nucleic acid sequence with SEQ ID NO: 12, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit has the nucleic acid sequence of SEQ ID NO: 12.

In one embodiment, the dimerization unit comprises or consists of the dHLX protein, e.g. the dHLX protein comprising or consisting of the nucleic acid sequence with SEQ ID NO: 120 or the amino acid sequence encoded by said nucleic acid sequence.

In the polypeptide encoded by the nucleotide sequence of the anticancer vaccine for use in the method of the invention, the multimerization unit, such as dimerization unit, may have any orientation with respect to antigenic unit and targeting unit. In one embodiment, the antigenic unit is connected to the C-terminal end of the multimerization/dimerization unit (e.g. via a unit linker) with the targeting unit being connected to the N-terminal end of the multimerization/dimerization unit. In another embodiment, the antigenic unit is connected to the N-terminal end of the multimerization/dimerization unit (e.g. via a unit linker) with the targeting unit being connected to the C-terminal end of the dimerization unit. It is preferred that the antigenic unit is connected to the C-terminal end of the multimerization/dimerization unit, preferably via the unit linker, and the targeting unit is connected to the N-terminal end of the multimerization/dimerization unit.

### Signal Peptide

In one embodiment, the anticancer vaccine for use in the method of the invention is a polynucleotide which further comprises a nucleotide sequence encoding a signal peptide. The signal peptide is either located at the N-terminal end of the targeting unit or the C-terminal end of the targeting unit, depending on the orientation of the targeting unit in the polypeptide. The signal peptide is designed to allow secretion of the polypeptide encoded by the polynucleotide in the cells transfected with said polynucleotide.

Any suitable signal peptide may be used. Examples of suitable peptides are an Ig VH signal peptide, such as SEQ ID NO: 2; a human TPA signal peptide, such as SEQ ID NO: 3 and a human MIP-1α signal peptide.

In one embodiment, the anticancer vaccine for use in the method of the invention is a polynucleotide which comprises a nucleotide sequence encoding a signal peptide that comprises an amino acid sequence having at least 85% sequence identity to the amino acid sequence 1-23 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In another preferred embodiment, the anticancer vaccine for use in the method of the invention is a polynucleotide which comprises a nucleotide sequence encoding a signal peptide that comprises the amino acid sequence 1-23 of SEQ ID NO: 1, except that at the most four amino acids have been substituted, deleted or inserted, such as at the most three amino acids, such as at the most two amino acids or such as at the most one amino acid.

In another preferred embodiment, the anticancer vaccine for use in the method of the invention is a polynucleotide which comprises a nucleotide sequence encoding a signal peptide that comprises the amino acid sequence 1-23 of SEQ ID NO: 1.

In a more preferred embodiment, the anticancer vaccine for use in the method of the invention is a polynucleotide which comprises a nucleotide sequence encoding a signal peptide that consists of an amino acid sequence having at least 80% sequence identity to the amino acid sequence 1-23 of SEQ ID NO: 1, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identify.

In another preferred embodiment, the anticancer vaccine for use in the method of the invention is a polynucleotide which comprises a nucleotide sequence encoding a signal peptide that consists of the amino acid sequence 1-23 of SEQ ID NO: 1, except that at the most four amino acids have been substituted, deleted or inserted, such as at the most three amino acids, such as at the most two amino acids or such as at the most one amino acid.

In another preferred embodiment, the anticancer vaccine for use in the method of the invention is a polynucleotide which comprises a nucleotide sequence encoding a signal peptide with the amino acid sequence 1-23 of SEQ ID NO: 1.

In one preferred embodiment, the anticancer vaccine for use in the method of the invention is a polynucleotide which comprises a nucleotide sequence encoding a signal peptide, wherein said nucleotide sequence has at least 80% sequence identity to the nucleic acid sequence with SEQ ID NO: 4.

In a further preferred embodiment, the anticancer vaccine for use in the method of the invention is a polynucleotide which comprises a nucleotide sequence encoding a signal peptide, wherein said nucleotide sequence has at least 85% sequence identity to the nucleic acid sequence with SEQ ID NO: 4, such as at least 86% or at least 87%, 88%, 89%, 90%, 91%, 92%, 93%, 94%, 95%, 96%, 97%, 98% or at least 99% sequence identity.

In yet a further preferred embodiment, the anticancer vaccine for use in the method of the invention is a polynucleotide which comprises a nucleotide sequence encoding a signal peptide, wherein said nucleotide sequence is SEQ ID NO: 4.

### Sequence Identity

Sequence identity may be determined as follows: a high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used.

Amino acid sequence variants may be prepared by introducing appropriate changes into the nucleotide sequence encoding anticancer vaccine, or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person in the art. Any combination of deletion, insertion, and substitution can be made to arrive at the final construct, provided that the final construct possesses the desired characteristics. For example, deletions, insertions or substitutions of amino acid residues may produce a silent change and result in a functionally equivalent peptide/polypeptide.

Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e. like-for-like substitution such as basic for basic, acidic for acidic, polar for polar etc. and non-conservative substitutions, i.e. from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, aaline, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include; alpha* and alpha-disubstituted* amino acids, N-alkyl amino acids*, lactic acid*, halide derivatives of natural amino acids such as trifluorotyrosine*, p-CI-phenylalanine*, p-Br-phenylalanine*, p-I-phenylalanine*, L-allyl-glycine*, β-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid * and L-Phe (4-benzyl)*.

In the paragraph above, * indicates the hydrophobic nature of the substituting residue, whereas # indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or B-alanine residues. A further form of variation involves the presence of one or more amino acid residues in peptoid form.

### Polynucleotides

The anticancer vaccine for use in the method of the invention may be in the form of a polynucleotide, e.g. DNA or RNA, including genomic DNA, cDNA and mRNA, either double-stranded or single-stranded. In one embodiment, the anticancer vaccine is a DNA vaccine, i.e. the polynucleotide is a DNA.

In one embodiment, the polynucleotide is optimized to the species of the subject to which it is administered. For administration to a human, in one embodiment, the polynucleotide sequence is human codon optimized.

### VB10.NEO

In one embodiment, the anticancer vaccine is an individualized DNA anticancer vaccine comprising VB10.NEO, a DNA polynucleotide comprising a nucleotide sequence encoding a signal peptide, a human MIP-1alpha targeting unit that targets antigen-presenting cells, a human IgG3 dimerization unit, a glycine rich linker unit, and an antigenic unit comprising one or more neoepitopes. If the antigenic unit comprises more than one neoepitope, said neoepitopes are separated by linkers. A description of VB10.NEO can be found in, e.g., International Patent Application No. PCT/EP2017/050206 filed on Jan. 5, 2017 and published as WO 2017/118695A1. VB10.NEO consists of a constant part which comprises the signal peptide, the targeting unit that targets antigen-presenting cells, the dimerization unit, and the unit linker and a variable part which comprises the antigenic unit and is specifically designed for each patient to which VB10.NEO is administered, and which comprises one or more neoepitopes identified in the tumor of the patient who is going to be treated with VB10.NEO. Thus, the antigenic unit is designed specifically, and only for the patient who is treated or who is to be treated with the vaccine and comprises one or more cancer antigens that are patient-specific antigens or parts thereof, such antigens including neoantigens or patient-present shared cancer antigens. After patient specific, tumor specific mutations are identified, antigenic peptides comprising neoepitopes are selected and nucleic acids encoding the neoepitopes are included into the antigenic unit of VB10.NEO.

VB10.NEO comprises a nucleotide sequence encoding a signal peptide. The signal peptide is a human MIP-1α signal peptide and VB10.NEO comprises a nucleotide sequence encoding the amino acid sequence 1-23 of SEQ ID NO: 1. VB10.NEO comprises a signal peptide which consists of the nucleic acid sequence of SEQ ID NO: 4.

VB10.NEO comprises a nucleotide sequence encoding a targeting which comprises or consists of human MIP-1α (LD78B, CCL3L1), i.e., amino acids 24-93 of SEQ ID NO: 1. VB10.NEO comprises a human MIP-1α targeting unit which consists of the nucleic acid sequence of SEQ ID NO: 5.

VB10.NEO comprises a nucleotide sequence encoding a dimerization unit which comprises a hinge exon h1, a hinge exon h4, a dimerization unit linker and a CH3 domain of human IgG3. VB10.NEO comprises a nucleotide sequence encoding a dimerization unit which comprises or consists of the amino acid sequence 94-237 of SEQ ID NO: 1. VB10.NEO comprises a dimerization unit which has the nucleic acid sequence of SEQ ID NO: 12.

VB10.NEO comprises a nucleotide sequence encoding a unit linker as described herein, e.g. as described under the heading “unit linker”. VB10.NEO comprises a nucleotide sequence encoding a unit linker which comprises or consists of GLGGL (SEQ ID NO: 52) (nucleotide sequence with SEQ ID NO: 10) or GLSGL (SEQ ID NO: 109).

VB10.NEO comprises a nucleotide sequence encoding a polypeptide which consists of the amino acid sequence 1-242 of SEQ ID NO: 1, i.e., the constant part of VB10.NEO consists of a nucleotide sequence encoding a polypeptide which consists of the amino acid sequence 1-242 of SEQ ID NO: 1. VB10.NEO comprises the nucleic acid sequence of SEQ ID NO: 23, i.e., the constant part of VB10.NEO consists of the nucleic acid sequence of SEQ ID NO: 23.

In one embodiment, VB10.NEO is comprised in a vector suitable for expression of a polypeptide or dimeric protein encoded by the polynucleotide, i.e., an expression vector, such as a DNA plasmid. The term “VB10.NEO” may be used for the afore-described DNA polynucleotide but may also be used for an expression vector, such as a DNA plasmid, comprising the afore-described DNA polynucleotide.

### Polypeptides and Multimeric/Dimeric Proteins

The anticancer vaccine for use in the method of the invention may be in the form of a polypeptide encoded by the polynucleotide as described above. The polypeptide may be expressed in vitro for production of the anticancer vaccine, or the polypeptide may be expressed in vivo as a result of the administration of the polynucleotide to a subject, as described above.

Due to the presence of the multimerization/dimerization unit, multimeric/dimeric proteins are formed when the polypeptide is expressed.

The multimeric/dimeric proteins may be homomultimers or hetereomultimers, e.g. if the protein is a dimeric protein, the dimeric protein may be a homodimer, i.e. a dimeric protein wherein the two polypeptide chains are identical and consequently comprise identical units and thus antigen sequences, or the dimeric protein may be a heterodimer comprising two polypeptide chains, wherein polypeptide chain 1 comprises different antigen sequences in its antigenic unit than polypeptide 2. The latter may be relevant if the number of antigens for inclusion into the antigenic unit would exceed an upper size limit for the antigenic unit. It is preferred that the dimeric protein is a homodimeric protein.

### Vectors

The polynucleotide sequence of the anticancer vaccine may be a DNA polynucleotide comprised in a vector suitable for transfecting a host cell and expression of a polypeptide or multimeric/dimeric protein encoded by the polynucleotide, i.e. an expression vector, such as a DNA plasmid. In another embodiment, the vector is suitable for transfecting a host cell and expression of an mRNA encoding for the polypeptide/multimeric protein.

In one embodiment, the vector allows for easy exchange of the various units described above, particularly the antigenic unit in case of individualized anticancer vaccines.

In one embodiment, the vector is a pUMVC4a vector or a vector comprising NTC9385R vector backbones. The antigenic unit may be exchanged with an antigenic unit cassette restricted by the SfiI restriction enzyme cassette where the 5′ site is incorporated in the nucleotide sequence encoding the GLGGL (SEQ ID NO: 52)/GLSGL (SEQ ID NO: 109) unit linker and the 3′ site is included after the stop codon in the vector.

Thus, the invention provides a method for treating a subject (e.g. a patient) having cancer, the method comprising administering to the subject
(a) an anticancer vaccine comprising a vector comprising a polynucleotide comprising a nucleotide sequence encoding a targeting unit that targets antigen-presenting cells, a multimerization unit, such as dimerization unit and an antigenic unit comprising one or more cancer antigens; and
(b) one or more checkpoint inhibitors.

In one embodiment, invention provides a method for treating a subject (e.g. a patient) having cancer, the method comprising administering to the subject
(a) an anticancer vaccine comprising a vector comprising a polynucleotide comprising a nucleotide sequence encoding a targeting unit that targets antigen-presenting cells, a multimerization unit and an antigenic unit comprising one or more cancer antigens; and
(b) one or more checkpoint inhibitors.

In another embodiment, the invention provides a method for treating a subject (e.g. a patient) having cancer, the method comprising administering to the subject
(a) an anticancer vaccine comprising a vector comprising a polynucleotide comprising a nucleotide sequence encoding a targeting unit that targets antigen-presenting cells, a dimerization unit and an antigenic unit comprising one or more cancer antigens; and
(b) one or more checkpoint inhibitors.

In a preferred embodiment, the vector is a DNA plasmid and the polynucleotide is DNA.

### Vaccines

The anticancer vaccine for use in the method of the invention may comprise a pharmaceutically acceptable carrier or diluent, including but not limited to saline, buffered saline, such as PBS, dextrose, water, glycerol, ethanol, sterile isotonic aqueous buffers, and combinations thereof.

In one embodiment, the pharmaceutically acceptable carrier or diluent is an aqueous buffer. In another embodiment, the aqueous buffer is Tyrode's buffer, e.g. Tyrode's buffer comprising 140 mM NaCl, 6 mM KCl, 3 mM CaCl2, 2 mM MgCl2, 10 mM 4-(2-hydroxyethyl)-1-piperazineethanesulfonic acid (Hepes) pH 7.4, and 10 mM glucose.

The anticancer vaccine may further comprise an adjuvant. Particularly for anticancer vaccines comprising polypeptides/proteins, pharmaceutically acceptable adjuvants include, but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1 P321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, resiquimod, SRL172, virosomes and other virus-like particles, YF-17D, VEGF trap, R848, beta-glucan, Pam3Cys, Aquila's QS21 stimulon, vadimezan, and/or AsA404 (DMXAA). However, as detailed above, due to the presence of the targeting unit, the anticancer vaccines for use in the method of the invention can be administered without additional adjuvant; thus, in some embodiments, the anticancer vaccine does not comprise an adjuvant.

In some specific embodiments the anticancer vaccine may comprise a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively.

This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO)4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In one embodiment, the anticancer vaccine comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In another embodiment, the anticancer vaccine comprises the amphiphilic block co-polymer in an amount of from 2% w/v to 5% w/v, such as about 3% w/v.

For anticancer vaccines comprising polynucleotides, the vaccines may further comprise molecules that ease transfection of cells.

The anticancer vaccine may be formulated in any way suitable administration to a subject, e.g. patient, such as a liquid formulation for injection, e.g. for intradermal or intramuscular injection.

The anticancer vaccine for use in the method of the invention may be administered in any way suitable for administration to a subject, e.g. a patient, of either a polypeptide/protein vaccine or a polynucleotide vaccine, such as administered by intradermal, intramuscular, intranodal or subcutaneous injection, or by mucosal or epithelial application, such as intranasal, oral, enteral or intravesicular (to the bladder) administration.

In a preferred embodiment, the anticancer vaccine comprises a polynucleotide as described herein, preferably a polynucleotide and a pharmaceutically acceptable carrier and is administered by intramuscular or intradermal injection.

The anticancer vaccine for use in the method of the invention typically comprises the polynucleotide in a range of from 0.3 mg to 6 mg, e.g. about 2 mg and the polypeptide/protein in the range of 5 μg to 5 mg.

### Methods for Preparing the Anticancer Vaccine for Use in the Method of the Invention

Suitable methods are disclosed in WO 2004/076489A1, WO 2011/161244A1, WO 2013/092875A1, WO 2017/118695A1 and WO 2021/205027 A1, which are incorporated herein by reference, in particular page 15, lines 10-13 and page 17, section “Construction of Vaccibodies” of WO 2004/076489A1; page 10, lines 10-14 and Example 1 of WO 2011/161244A1; page 15 and Example 1 of WO 2013/092875A1; section “Methods for preparing the vaccine” and Example 1 of WO 2017/118695A1 and page 26, line 17 to page 27, line 38, page 30, line 23 to page 31, line 27 and Example 4 of WO 2021/205027 A1.

### Checkpoint Inhibitors

The method of the invention comprises administration of one or more checkpoint inhibitors. Checkpoint inhibitors suitable for use in the method provided herein include antibodies, such as for example, monoclonal antibodies, that target PD-1, PD-L1, CTLA-4 or TIGIT.

Exemplary anti-PD1 inhibitors, e.g. anti-PD1 antibodies include, but are not limited to pembrolizumab, nivolumab, and cemiplimab. Representative anti-PD-L1 inhibitors, e.g anti-PD-L1 antibodies, include, for example, atezolizumab, avelumab, and durvalumab. Exemplary anti-CTLA-4 inhibitors, e.g. anti-CTLA4 antibodies, include, for example ipilimumab or tremelimumab. Representative anti-TIGIT inhibitors, e.g. anti-TIGIT antibodies, include, for example, tiragolumab, BMS-986207, ociperlimab, COM902, AB154, EOS884448, IBI939, SGNTNT and etigilimab.

Preferably, the checkpoint inhibitor is selected from the group consisting of anti-PD-L1 antibody, anti-TIGIT antibody, anti-CTLA-4-antibody and anti-PD1-antibody.

In one embodiment, the checkpoint inhibitor is an anti-PD-L1 antibody, e.g., atezolizumab.

The term “hypervariable region” or “HVR” as used herein refers to each of the regions of an antibody variable domain which are hypervariable in sequence and which determine antigen binding specificity, for example “complementarity determining regions” (“CDRs”). Generally, antibodies comprise six CDRs: three in the heavy chain variable region (VH) (CDR-H1, CDR-H2, CDR-H3), and three in the light chain variable region (VL) (CDR-L1, CDR-L2, CDR-L3).

In some embodiments, the anti-PD-L1 antibody comprises atezolizumab which comprises:
(a) the heavy chain amino acid sequence of SEQ ID NO: 13:, and
(b) the light chain amino acid sequence of SEQ ID NO: 14.

In some embodiments, the anti-PD-L1 antibody comprises:
(a) an HVR-H1, HVR-H2, and HVR-H3 sequence of GFTFSDSWIH (SEQ ID NO: 15), AWISPYGGSTYYADSVKG (SEQ ID NO: 16) and RHWPGGFDY (SEQ ID NO: 17), respectively, and
(b) an HVR-L1, HVR-L2, and HVR-L3 sequence of RASQDVSTAVA (SEQ ID NO: 18), SASFLYS (SEQ ID NO: 19) and QQYLYHPAT (SEQ ID NO: 20), respectively.

The HVR-H1, HVR-H2, HVR-H3, HVR-L1, HVR-L2, and HVR-L3 sequences of atezolizumab are SEQ ID NOS: 15-20, respectively.

In some embodiments, the anti-PD-L1 antibody comprises:
(a) a heavy chain variable region (VH) comprising the amino acid sequence of SEQ ID NO: 21; and
(b) the light chain variable region (VL) comprising the amino acid sequence of SEQ ID NO: 22.

The VH of atezolizumab comprises SEQ ID NO: 21; the VL of atezolizumab comprises SEQ ID NO: 22.

Atezolizumab has been approved in many countries as monotherapy for the treatment of certain cancers. For example, atezolizumab has been approved in the USA or Europe for the following indications: treatment of adult patients with locally advanced or metastatic urothelial carcinoma (UC) after prior platinum-containing chemotherapy, or who are considered cisplatin ineligible and whose tumors have a PD-L1 expression ≥5%, treatment of adult patients with locally advanced or metastatic non-small cell lung cancer (NSCLC) after prior chemotherapy; treatment of patients with locally advanced or metastatic UC who are not eligible for cisplatin-containing chemotherapy and whose tumors express PD-L1 (PD-L1 stained ICs covering ≥5% of the tumor area), or are not eligible for any platinum-containing chemotherapy regardless of level of tumor PD-L1 expression, or have disease progression during or after any platinum-containing chemotherapy or within 12 months of neoadjuvant or adjuvant chemotherapy; and treatment of patients with metastatic NSCLC who have disease progression during or after platinum-containing chemotherapy. Atezolizumab is also undergoing development as monotherapy and in combination with other targeted and cytotoxic agents for the treatment of patients with multiple solid and hematological tumors, including lung, renal, colorectal, and breast cancers.

All currently approved indications for atezolizumab are approved at a dose of 1200 mg as an intravenous (IV) infusion every 3 weeks (Q3W) until disease progression or unacceptable toxicity occurs.

In another embodiment, the checkpoint inhibitor is an anti-TIGIT antibody. In yet another embodiment, the checkpoint inhibitor is an anti-CTLA-4-antibody. In yet another embodiment, the checkpoint inhibitor is an anti-PD1-antibody.

In one embodiment, the method of the invention comprises the administration of two checkpoint inhibitors, e.g. the administration of an anti-PD-L1 antibody and an anti-TIGIT antibody or the administration of an anti-PD-L1 antibody and an anti-CTLA-4-antibody or the administration of an anti-PD-1 antibody and an anti-TIGIT antibody or the administration of an anti-PD-1 antibody and an anti-CTLA-4-antibody or the administration of an anti-TIGIT antibody and an anti-CTLA-4-antibody or the administration of an anti-PD-L1 antibody and an anti-PD-1 antibody.

In another embodiment, the method of the invention comprises the administration of three checkpoint inhibitors, e.g. the administration of an anti-PD-L1 antibody, an anti-PD-1 antibody and either an anti-CTLA-4-antibody or an anti-TIGIT antibody or the administration of an anti-PD-L1 antibody, an anti-CTLA-4-antibody and an anti-TIGIT antibody or the administration of an anti-PD-1 antibody, an anti-CTLA-4-antibody and an anti-TIGIT antibody.

In yet another embodiment, the method of the invention comprises the administration of four checkpoint inhibitors, e.g. the administration of an anti-PD-L1 antibody, an anti-PD-1 antibody, an anti-CTLA-4-antibody and an anti-TIGIT antibody.

In a preferred embodiment, the method of the invention comprises the administration of an anti-TIGIT antibody or anti-PD-L1 antibody or anti-CTLA-4 antibody. In another preferred embodiment, the method of the invention comprises the administration of an anti-TIGIT antibody and anti-PD-L1 antibody or the administration of an anti-CTLA-4 antibody and anti-PD-L1 antibody.

In one embodiment, the checkpoint inhibitor, e.g. each checkpoint inhibitor, is comprised in a composition suitable for injection. Such composition may be a liquid composition suitable for injection, e.g. infusion injection, comprising e.g. sterile water, saline, such as isotonic saline, a sugar solution for intravenous administration, such as a dextrose solution, an electrolyte solution for intravenous administration, such as Ringer's lactate or Ringer's acetate or an aqueous buffer.

In another embodiment, several checkpoint inhibitors are comprised in a composition suitable for injection, such as a liquid composition suitable for injection, e.g. two, three or four checkpoint inhibitors.

The composition may comprise the checkpoint inhibitor or several checkpoint inhibitors in a total amount of from about 0.1 mg/kg to about 1 mg/kg, e.g. from about 0.2 mg/kg to about 0.9 mg/kg, from about 0.3 mg/kg to about 0.8 mg/kg, form about 0.4 mg/kg to about 0.7 mg/kg, from about 0.5 mg/kg to about 0.6 mg/kg or from about 1 mg/kg to about 1000 mg/kg, e.g. from about 2 mg/kg to about 900 mg/kg; from about 3 mg/kg to about 800 mg/kg; from about 4 mg/kg to about 700 mg/kg; from about 5 mg/kg to about 600 mg/kg; from about 6 mg/kg to about 550 mg/kg; from about 7 mg/kg to about 500 mg/kg; from about 8 mg/kg to about 450 mg/kg; from about 9 mg/kg to about 400 mg/kg; from about 5 mg/kg to about 200 mg/kg; from about 2 mg/kg to about 150 mg/kg; from about 5 mg/kg to about 100 mg/kg; from about 10 mg/kg to about 100 mg/kg; and from about 10 mg/kg to about 60 mg/kg.

Further guidance may be provided based upon the current standard of care for administration of checkpoint inhibitors in cancer therapy.

In one embodiment, a composition comprising the checkpoint inhibitor (e.g. a composition as described above) is administered via injection. Injection includes subcutaneous, intravenous, intra-arterial, intratumoral, intralymphatic, intraperitoneal, intracardiac, intrathecal, and intramuscular injection, as well as infusion injections.

### Treatment

In one embodiment, the subject that is treated according to the method of the invention has received one or more anticancer treatments prior to be treated according to the method of the invention. Such anticancer treatments include surgery, radiation therapy, chemotherapy and immunotherapy. In another embodiment, the subject is treatment-naïve. In some embodiments, the cancer is non-metastatic. In other embodiments, the cancer is metastatic. In one embodiment, the subject has received a prior therapy to treat the cancer and the cancer treated by the method of the invention is recurrent, relapsed or refractory to the prior therapy.

With regard to the frequency and schedule of administering the anticancer vaccine and checkpoint inhibitor in the method of the invention, one of ordinary skill in the art will be able to determine an appropriate frequency and schedule for each of them.

For example, in a treatment cycle, a clinician can decide to administer the anticancer vaccine, either as a single dose or in repetitive doses, i.e. in a series of doses, e.g. over the course of several days, weeks or months.

The anticancer vaccine may be the same for each administration, or different anticancer vaccines may be administered, e.g. different anticancer vaccines in different treatment cycles. As an example, an individualized anticancer vaccine comprising a polynucleotide encoding 20 neoepitopes (neoepitopes 1-20 with sequence 1-20) or comprising a polypeptide/protein comprising said 20 neoepitopes is administered to a patient in a series of doses, e.g. over the course of several days, weeks or months. The neoepitopes 1-20 are a subgroup of a total of 60 different neoepitopes that had been identified in said patient, of which neoepitopes 1-20 were selected by methods and based on criteria as described earlier in this application for inclusion into the vaccine. In a subsequent treatment cycle, another individualized anticancer vaccine is administered to the same patient, comprising a polynucleotide encoding 20 neoepitopes (neoepitopes 21-40 with sequence 21-40) or comprising a polypeptide/protein comprising said 20 neoepitopes in a series of doses, e.g. over the course of several days, weeks or months. The neoepitopes 21-40 are also a subgroup of the total of 60 different neoepitopes that had been identified in said patient.

In one embodiment, the checkpoint inhibitor is administered concurrently with the anticancer vaccine, e.g. on the same day or the same time of the same day, either as a single dose or in repetitive doses, i.e. in a series of doses, e.g. over the course of several days, weeks or months. Thus, the first administration of one or more checkpoint inhibitors and the first administration of the anticancer vaccine is done concurrently, e.g. on the same day or the same time of the same day.

In another embodiment, the checkpoint inhibitor is administered prior to the anticancer vaccine, either as a single dose or in repetitive doses, i.e. in a series of doses, e.g. over the course of several days, weeks or months. Thus, the first administration of one or more checkpoint inhibitors is done prior to the first administration of the anticancer vaccine.

In yet another embodiment, the anticancer vaccine is administered prior to the one or more checkpoint inhibitors, either as a single dose or in repetitive doses, i.e. in a series of doses, e.g. over the course of several days, weeks or months. Thus, the first administration of the anticancer vaccine is done prior to the first administration of the one or more checkpoint inhibitors.

In one embodiment, the method of the invention further includes one or more of the following steps: identifying neoepitopes and/or patient-present shared antigens in a patient, designing the antigenic unit for said patient and manufacturing of the individualized anticancer vaccine for said patient, all of these steps are carried out prior to the administration of the individualized anticancer vaccine.

In another embodiment, the method of the invention includes a vaccination induction period, where the anticancer vaccine is administered more frequently and/or at a higher dose, and a vaccination maintenance period where the anticancer vaccine is administered less frequently and/or at a lower dose.

The actual dose for each of the anticancer vaccine and the checkpoint inhibitor to be administered in the method according to the invention will vary and depend on the age, weight, and general condition of the subject, the severity of the cancer being treated, the judgment of the health care professionals and the particular nature and properties of each of the anticancer vaccine and the checkpoint inhibitor(s).

Exemplary lengths of time associated with the method of treating according to the invention include about 1-24 weeks, or about 6 to 12 months, or about 1 to 5 years.

The method of treating according to the invention can continue for as long as the clinician overseeing the patient's care deems the method to be effective and the treatment to be needed. Non-limiting parameters that indicate that the method is effective include any one or more of the following: reduction in disease progression or stable disease, i.e. the cancer does progress at a slower rate or does not progress. This includes that a tumor does grow at a slower rate or does not grow and/or does spread slower or does not spread, e.g. to lymph nodes or forming metastases and/or does not become more aggressive. Other non-limiting parameters that indicate that the method is effective include tumor shrinkage (in terms of weight and/or volume); a decrease in the number of individual tumor colonies; tumor elimination; and progression-free survival.

A response to the method of treating according to the invention may be evaluated at any suitable time point during the treatment, e.g. after a single round of treatment, after 2-3 cycles of treatment, etc., and by any of a number of suitable methods, including shrinkage of a tumor (partial response), i.e., an evaluation of tumor size or volume, disappearance of a tumor, a reduction in disease progression or a stable disease, and analysis of one or more tumor test markers if appropriate.

In another embodiment, the method according to the invention is effective to inhibit accumulation of regulatory T cells in a subject or to stimulate T cell and/or NK cell activity and/or proliferation of such cells in a subject.

Methods to determine whether a patient has responded to the method of the invention are known in the art. Change in tumor size may be determined by any suitable method such as imaging. Various diagnostic imaging modalities can be employed, such as computed tomography (CT scan), dual energy CDT, positron emission tomography and MRI. Other secondary indicators for treatment success include an appropriate response by a suitable tumor marker (if applicable), increased number of NK (natural killer) cells, increased number of T cells or reduced numbers of regulatory T cells.

The method according to the invention may be the main method of treating a patient's cancer or may be an adjuvant method. For example, the main method of treatment may be surgery to remove a tumor, and the method of the invention is used as an adjuvant treatment prior and/or after such surgery, either alone or in combination with other method of treatments, like chemotherapy or radiation therapy.

The method according to the invention is for treating a patient with cancer. For example, patients may be responsive to the anticancer vaccine alone or to the checkpoint inhibitor alone, but are more responsive to administration of the combination. By way of further example, patients may be only marginally responsive to the anticancer vaccine, or only marginally responsive to the checkpoint inhibitor, but are responsive or more responsive to the combination.

The method according to the invention can be used for treating a patient having a solid cancer or liquid cancer. Examples of solid cancers are cancers forming a solid mass, e.g. a tumor. Examples of liquid cancers are cancers present in body fluid, such as lymphomas or blood cancers. Examples of cancers that can be treated with the method according to the invention are breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

### Also Disclosed Herein is:

An anticancer vaccine comprising (i) a polynucleotide comprising a nucleotide sequence encoding a targeting unit that targets antigen-presenting cells, a multimerization unit, such as a dimerization unit, and an antigenic unit comprising one or more cancer antigens; or (ii) a polypeptide encoded by the polynucleotide as defined in (i); or (iii) a multimeric protein, such as a dimeric protein consisting of multiple polypeptides as defined in (ii), such as of two polypeptides; and one or more checkpoint inhibitors for use in the treatment of a subject having cancer, wherein the anticancer vaccine and the one or more checkpoint inhibitors are administered to said subject.

### Further, Also Disclosed Herein is:

An anticancer vaccine comprising (i) a polynucleotide comprising a nucleotide sequence encoding a targeting unit that targets antigen-presenting cells, a multimerization unit and an antigenic unit comprising one or more cancer antigens; or (ii) a polypeptide encoded by the polynucleotide as defined in (i); or (iii) a multimeric protein consisting of multiple polypeptides as defined in (ii); and one or more checkpoint inhibitors for use in the treatment of a subject having cancer, wherein the anticancer vaccine and the one or more checkpoint inhibitors are administered to said subject.

### Further, Also Disclosed Herein is:

An anticancer vaccine comprising (i) a polynucleotide comprising a nucleotide sequence encoding a targeting unit that targets antigen-presenting cells, a dimerization unit and an antigenic unit comprising one or more cancer antigens; or (ii) a polypeptide encoded by the polynucleotide as defined in (i); or (iii) a dimeric protein consisting of two polypeptides as defined in (ii); and one or more checkpoint inhibitors for use in the treatment of a subject having cancer, wherein the anticancer vaccine and the one or more checkpoint inhibitors are administered to said subject.

The anticancer vaccine, the one or more checkpoint inhibitors and their respective administration to the subject are described in detail earlier in this application.

### Further, Also Disclosed Herein is the:

Use of (a) an anticancer vaccine comprising (i) a polynucleotide comprising a nucleotide sequence encoding a targeting unit that targets antigen-presenting cells, a multimerization unit, such as a dimerization unit, and an antigenic unit comprising one or more cancer antigens; or (ii) a polypeptide encoded by the polynucleotide as defined in (i); or (iii) a multimeric protein, such as a dimeric protein, consisting of multiple polypeptides as defined in (ii), such as of two polypeptides; and (b) one or more checkpoint inhibitors for the manufacture of a medicament for the treatment of a subject having cancer, wherein the medicament is administered to said subject.

### Further, Also Disclosed Herein is the:

Use of (a) an anticancer vaccine comprising (i) a polynucleotide comprising a nucleotide sequence encoding a targeting unit that targets antigen-presenting cells, a multimerization unit and an antigenic unit comprising one or more cancer antigens; or (ii) a polypeptide encoded by the polynucleotide as defined in (i); or (iii) a multimeric protein consisting of multiple polypeptides as defined in (ii); and (b) one or more checkpoint inhibitors for the manufacture of a medicament for the treatment of a subject having cancer, wherein the medicament is administered to said subject.

### Further, Also Disclosed Herein is the:

Use of (a) an anticancer vaccine comprising (i) a polynucleotide comprising a nucleotide sequence encoding a targeting unit that targets antigen-presenting cells, a dimerization unit and an antigenic unit comprising one or more cancer antigens; or (ii) a polypeptide encoded by the polynucleotide as defined in (i); or (iii) a dimeric protein consisting of two polypeptides as defined in (ii); and (b) one or more checkpoint inhibitors for the manufacture of a medicament for the treatment of a subject having cancer, wherein the medicament is administered to said subject.

The anticancer vaccine, the one or more checkpoint inhibitors and their respective administration to the subject are described in detail earlier in this application.

### Further, Also Disclosed Herein is the:

Use of (a) an anticancer vaccine comprising (i) a polynucleotide comprising a nucleotide sequence encoding a targeting unit that targets antigen-presenting cells, a multimerization unit, such as a dimerization unit, and an antigenic unit comprising one or more cancer antigens; or (ii) a polypeptide encoded by the polynucleotide as defined in (i); or (iii) a multimeric protein, such as a dimeric protein, consisting of multiple polypeptides as defined in (ii), such as of two polypeptides; and (b) one or more checkpoint inhibitors for treating a subject having cancer.

### Further, Also Disclosed Herein is the:

Use of (a) an anticancer vaccine comprising (i) a polynucleotide comprising a nucleotide sequence encoding a targeting unit that targets antigen-presenting cells, a multimerization unit and an antigenic unit comprising one or more cancer antigens; or (ii) a polypeptide encoded by the polynucleotide as defined in (i); or (iii) a multimeric protein consisting of multiple polypeptides as defined in (ii); and (b) one or more checkpoint inhibitors for treating a subject having cancer.

### Further, Also Disclosed Herein is the:

Use of (a) an anticancer vaccine comprising (i) a polynucleotide comprising a nucleotide sequence encoding a targeting unit that targets antigen-presenting cells, a dimerization unit and an antigenic unit comprising one or more cancer antigens; or (ii) a polypeptide encoded by the polynucleotide as defined in (i); or (iii) a dimeric protein consisting of two polypeptides as defined in (ii); and (b) one or more checkpoint inhibitors for treating a subject having cancer.

The anticancer vaccine, the one or more checkpoint inhibitors and the treatment are described in detail earlier in this application.

### Further, Also Disclosed Herein is:

An anticancer vaccine comprising (i) a polynucleotide comprising a nucleotide sequence encoding a targeting unit that targets antigen-presenting cells, a multimerization unit, such as a dimerization unit and an antigenic unit comprising one or more cancer antigens; or (ii) a polypeptide encoded by the polynucleotide as defined in (i); or (iii) a multimeric protein, such as a dimeric protein, consisting of multiple polypeptides as defined in (ii), such as of two polypeptides; and one or more checkpoint inhibitors, when used in the treatment of cancer.

### Further, Also Disclosed Herein is:

An anticancer vaccine comprising (i) a polynucleotide comprising a nucleotide sequence encoding a targeting unit that targets antigen-presenting cells, a multimerization unit and an antigenic unit comprising one or more cancer antigens; or (ii) a polypeptide encoded by the polynucleotide as defined in (i); or (iii) a multimeric protein consisting of multiple polypeptides as defined in (ii); and one or more checkpoint inhibitors, when used in the treatment of cancer.

### Further, Also Disclosed Herein is:

An anticancer vaccine comprising (i) a polynucleotide comprising a nucleotide sequence encoding a targeting unit that targets antigen-presenting cells, a dimerization unit and an antigenic unit comprising one or more cancer antigens; or (ii) a polypeptide encoded by the polynucleotide as defined in (i); or (iii) a dimeric protein consisting of two polypeptides as defined in (ii); and one or more checkpoint inhibitors, when used in the treatment of cancer.

The anticancer vaccine, the one or more checkpoint inhibitors and the treatment are described in detail earlier in this application.

### Further, Also Disclosed Herein is the:

Use of (a) an anticancer vaccine comprising (i) a polynucleotide comprising a nucleotide sequence encoding a targeting unit that targets antigen-presenting cells, a multimerization unit, such as a dimerization unit and an antigenic unit comprising one or more cancer antigens; (ii) a polypeptide encoded by the polynucleotide as defined in (i); or (iii) a multimeric protein, such as a dimeric protein, consisting of multiple polypeptides as defined in (ii); and (b) one or more checkpoint inhibitors for treatment of cancer.

### Further, Also Disclosed Herein is the:

Use of (a) an anticancer vaccine comprising (i) a polynucleotide comprising a nucleotide sequence encoding a targeting unit that targets antigen-presenting cells, a multimerization unit and an antigenic unit comprising one or more cancer antigens; (ii) a polypeptide encoded by the polynucleotide as defined in (i); or (iii) a multimeric protein consisting of multiple polypeptides as defined in (ii); and (b) one or more checkpoint inhibitors for treatment of cancer.

### Further, Also Disclosed Herein is the:

Use of (a) an anticancer vaccine comprising (i) a polynucleotide comprising a nucleotide sequence encoding a targeting unit that targets antigen-presenting cells, a dimerization unit and an antigenic unit comprising one or more cancer antigens; (ii) a polypeptide encoded by the polynucleotide as defined in (i); or (iii) a dimeric protein consisting of two polypeptides as defined in (ii); and (b) one or more checkpoint inhibitors for treatment of cancer.

The anticancer vaccine, the one or more checkpoint inhibitors and the treatment are described in detail earlier in this application.

### Further, Also Disclosed Herein is:

A medicament for the treatment of cancer in a subject having cancer by administering to the subject (a) an anticancer vaccine comprising (i) a polynucleotide comprising a nucleotide sequence encoding a targeting unit that targets antigen-presenting cells, a multimerization unit, such as a dimerization unit, and an antigenic unit comprising one or more cancer antigens; (ii) a polypeptide encoded by the polynucleotide as defined in (i); or (iii) a multimeric protein, such as a dimeric protein, consisting of multiple polypeptides as defined in (ii), such as of two polypeptides; and (b) one or more checkpoint inhibitors.

### Further, Also Disclosed Herein is:

A medicament for the treatment of cancer in a subject having cancer by administering to the subject (a) an anticancer vaccine comprising (i) a polynucleotide comprising a nucleotide sequence encoding a targeting unit that targets antigen-presenting cells, a multimerization unit and an antigenic unit comprising one or more cancer antigens; (ii) a polypeptide encoded by the polynucleotide as defined in (i); or (iii) a multimeric protein consisting of multiple polypeptides as defined in (ii); and (b) one or more checkpoint inhibitors.

### Further, Also Disclosed Herein is:

A medicament for the treatment of cancer in a subject having cancer by administering to the subject (a) an anticancer vaccine comprising (i) a polynucleotide comprising a nucleotide sequence encoding a targeting unit that targets antigen-presenting cells, a dimerization unit and an antigenic unit comprising one or more cancer antigens; (ii) a polypeptide encoded by the polynucleotide as defined in (i); or (iii) a dimeric protein consisting of two polypeptides as defined in (ii); and (b) one or more checkpoint inhibitors.

The anticancer vaccine, the one or more checkpoint inhibitors, their respective administration to the subject and the treatment are described in detail earlier in this application.

### Kit

In another aspect, the invention provides a kit comprising
(a) an anticancer vaccine comprising
(i) a polynucleotide comprising a nucleotide sequence encoding a targeting unit that targets antigen-presenting cells, a multimerization unit, such as a dimerization unit, and an antigenic unit comprising one or more cancer antigens; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

In one embodiment, the invention provides a kit comprising
(a) an anticancer vaccine comprising
(i) a polynucleotide comprising a nucleotide sequence encoding a targeting unit that targets antigen-presenting cells, a multimerization unit and an antigenic unit comprising one or more cancer antigens; or
(ii) a polypeptide encoded by the polynucleotide as defined in (i); or
(iii) a multimeric protein consisting of multiple polypeptides as defined in (ii); and
(b) one or more checkpoint inhibitors.

In another embodiment, the invention provides a kit comprising
(a) an anticancer vaccine comprising
(i) a polynucleotide comprising a nucleotide sequence encoding a targeting unit that targets antigen-presenting cells, a dimerization unit and an antigenic unit comprising one or more cancer antigens; or
(ii) a polypeptide encoded by the polynucleotide as defined in (i); or
(iii) a dimeric protein consisting of two polypeptides as defined in (ii); and
(b) one or more checkpoint inhibitors.

In yet another embodiment, the invention provides a kit comprising
(a) an anticancer vaccine comprised in one or more containers, wherein the anticancer vaccine comprises
(i) a polynucleotide comprising a nucleotide sequence encoding a targeting unit that targets antigen-presenting cells, a multimerization unit, such as a dimerization unit, and an antigenic unit comprising one or more cancer antigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors comprised in one or more second containers.

Said kit may further comprise one or more third containers comprising pharmaceutically acceptable carriers or diluents, such as carriers and diluents for reconstituting a dose of (a) and/or (b).

In yet another embodiment, the invention provides a kit comprising
(a) an anticancer vaccine comprised in one or more first containers wherein the anticancer vaccine comprises
(i) a polynucleotide comprising a nucleotide sequence encoding a targeting unit that targets antigen-presenting cells, a multimerization unit, and an antigenic unit comprising one or more cancer antigens or parts thereof; or
(ii) a polypeptide encoded by the polynucleotide as defined in (i); or
(iii) a multimeric protein consisting of multiple polypeptides as defined in (ii); and
(b) one or more checkpoint inhibitors comprised in one or more second containers.

Said kit may further comprise one or more third containers comprising pharmaceutically acceptable carriers or diluents, such as carriers and diluents for reconstituting a dose of (a) and/or (b).

In yet another embodiment, the invention provides a kit comprising
(a) an anticancer vaccine comprised in one or more first containers, wherein the anticancer vaccine comprises
(i) a polynucleotide comprising a nucleotide sequence encoding a targeting unit that targets antigen-presenting cells, a dimerization unit, and an antigenic unit comprising one or more cancer antigens or parts thereof; or
(ii) a polypeptide encoded by the polynucleotide as defined in (i); or
(iii) a dimeric protein consisting of two polypeptides as defined in (ii); and
(b) one or more checkpoint inhibitors comprised in one or more second containers.

Said kit may further comprise one or more third containers comprising pharmaceutically acceptable carriers or diluents, such as carriers and diluents for reconstituting a dose of (a) and/or (b).

The kit is for use in a method for treating a subject having cancer, e.g. a patient, wherein the method comprises the administration of the anticancer vaccine and administration of the one or more checkpoint inhibitors comprised in the kit to the subject.

The kit may further comprise instructions for use, including instructions for the reconstitution of a dose of (a) and (b) instructions for administration, and/or instructions for determining a suitable dose of (a) and (b), and/or instructions for the administration of (a) and (b), and/or the frequency and schedule of administering (a) and (b).

### Anticancer Vaccine Comprised in the Kit According to the Invention

The anticancer vaccine comprised in the kit is described in detail earlier in this application.

In a kit of a first embodiment, the anticancer vaccine comprised in the kit is provided in a first container comprising the polynucleotide/polypeptide/dimeric protein and a second container comprised in the kit comprises a pharmaceutically acceptable carrier or diluent, including but not limited to saline, buffered saline, such as PBS, dextrose, water, glycerol, ethanol, sterile isotonic aqueous buffers, and combinations thereof.

The kit further comprises instructions on how to combine the contents of the first and second container to reconstitute a dose of the anticancer vaccine for administration, prior to such administration.

In one embodiment, the kit comprises one such first and one such second container for reconstituting a single dose of the anticancer vaccine. In another embodiment, the kit comprises several such first and several such second containers for reconstituting several doses of the anticancer vaccine for repetitive dosing. In yet another embodiment, the kit comprises several such first containers and one such second container for reconstituting several doses of the anticancer vaccine for repetitive dosing, with the second container comprising sufficient content for the reconstitution of the anticancer vaccine comprised in the several first containers. The repetitive doses may be a series of doses, e.g. for use over the course of several days, weeks or months.

The containers comprised in the kit of the first embodiment may be any containers suitable for containing the anticancer vaccine or the pharmaceutically acceptable carrier.

In a kit of a second embodiment, the anticancer vaccine comprised in the kit is provided in a container, i.e. a single container, comprising a composition comprising the polynucleotide/polypeptide/dimeric protein and a pharmaceutically acceptable carrier or diluent, including but not limited to saline, buffered saline, such as PBS, dextrose, water, glycerol, ethanol, sterile isotonic aqueous buffers, and combinations thereof. As such, the anticancer vaccine may be in a form ready for administration or it may need to be further diluted with a pharmaceutically acceptable carrier or diluent, e.g. immediately before it is administered, which pharmaceutically acceptable carrier or diluent may be a further component in the kit.

The kit of the second embodiment may comprise one or more containers, e.g. one container for a single dose or several containers for repetitive doses, such as a series of doses, e.g. for use over the course of several days, weeks or months.

The container comprised in the kit of the second embodiment may be any container suitable for containing the anticancer vaccine and the pharmaceutically acceptable carrier.

The pharmaceutically acceptable carrier or diluent of the first embodiment or the anticancer vaccine of the second embodiment may further comprise an adjuvant. Particularly for anticancer vaccines comprising polypeptides/proteins, pharmaceutically acceptable adjuvants include, but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1 P321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, resiquimod, SRL172, virosomes and other virus-like particles, YF-17D, VEGF trap, R848, beta-glucan, Pam3Cys, Aquila's QS21 stimulon, vadimezan, and/or AsA404 (DMXAA).

For anticancer vaccines comprising polynucleotides, the pharmaceutically acceptable carrier or diluent or the anticancer vaccine may comprise molecules that ease transfection of cells.

The kit of the first and second embodiment preferably comprises instructions for the administration of the anticancer vaccine to a subject in need thereof, e.g. patient.

In one embodiment, the first container of the kit of the first embodiment or the single container of the kit of the second embodiment comprises an anticancer vaccine comprising a polynucleotide, and the second container of the kit of the first embodiment or the single container of the kit of the second embodiment comprises a pharmaceutically acceptable carrier. In a further preferred embodiment, the kit of said first and second embodiment comprises instructions for the intramuscular or intradermal injection of said anticancer vaccine.

The first container of the kit of the first embodiment or the single container of the kit of the second embodiment typically comprises 0.3 mg to 6 mg, e.g. about 2 mg if the anticancer vaccine is a polynucleotide and 5 μg to 5 mg, if the anticancer vaccine is a polypeptide/dimeric protein.

### Checkpoint Inhibitor Comprised in the Kit According to the Invention

The one or more checkpoint inhibitors comprised in the kit are described in detail earlier in this application.

In one embodiment, the one or more checkpoint inhibitors comprised in the kit are provided in one or more containers, e.g. each container comprising one checkpoint inhibitor, or in one container comprising all of the checkpoint inhibitors. The kit further comprises one or more containers comprising a liquid suitable for injection, including infusion injection, e.g. sterile water, saline, such as isotonic saline, a sugar solution for intravenous administration, such as a dextrose solution, an electrolyte solution for intravenous administration, such as Ringer's lactate or Ringer's acetate or an aqueous buffer.

The kit preferably further comprises instructions on how to combine the contents of the container(s) comprising the checkpoint inhibitor(s) and the contents of the container(s) comprising the liquid suitable for injection, including infusion injection, to obtain a composition comprising the checkpoint inhibitor(s), which is ready for administration.

The composition may comprise the checkpoint inhibitor or several checkpoint inhibitors in a total amount of from about 0.1 mg/kg to about 1 mg/kg, e.g. from about 0.2 mg/kg to about 0.9 mg/kg, from about 0.3 mg/kg to about 0.8 mg/kg, form about 0.4 mg/kg to about 0.7 mg/kg, from about 0.5 mg/kg to about 0.6 mg/kg or from about 1 mg/kg to about 1000 mg/kg, e.g. from about 2 mg/kg to about 900 mg/kg; from about 3 mg/kg to about 800 mg/kg; from about 4 mg/kg to about 700 mg/kg; from about 5 mg/kg to about 600 mg/kg; from about 6 mg/kg to about 550 mg/kg; from about 7 mg/kg to about 500 mg/kg; from about 8 mg/kg to about 450 mg/kg; from about 9 mg/kg to about 400 mg/kg; from about 5 mg/kg to about 200 mg/kg; from about 2 mg/kg to about 150 mg/kg; from about 5 mg/kg to about 100 mg/kg; from about 10 mg/kg to about 100 mg/kg; and from about 10 mg/kg to about 60 mg/kg.

The kit may comprise a number of containers suitable for a single dose of the one or more checkpoint inhibitors, or a number of containers suitable for repetitive doses of the one or more checkpoint inhibitors, such as a series of doses, e.g. for use over the course of several days, weeks or months.

In another embodiment, the kit comprises one or more containers comprising the one or more checkpoint inhibitors in a composition suitable for injection, such as infusion injection, e.g. each container comprises one checkpoint inhibitor in a composition suitable for injection, or one container comprises all of the checkpoint inhibitors in a composition suitable for injection. The composition suitable for injection comprises a suitable pharmaceutically acceptable carrier or diluent e.g. sterile water, saline, such as isotonic saline, a sugar solution for intravenous administration, such as a dextrose solution, an electrolyte solution for intravenous administration, such as Ringer's lactate or Ringer's acetate or an aqueous buffer.

The composition may comprise the checkpoint inhibitor or several checkpoint inhibitors in a total amount of from about 0.1 mg/kg to about 1 mg/kg, e.g. from about 0.2 mg/kg to about 0.9 mg/kg, from about 0.3 mg/kg to about 0.8 mg/kg, form about 0.4 mg/kg to about 0.7 mg/kg, from about 0.5 mg/kg to about 0.6 mg/kg or from about 1 mg/kg to about 1000 mg/kg, e.g. from about 2 mg/kg to about 900 mg/kg; from about 3 mg/kg to about 800 mg/kg; from about 4 mg/kg to about 700 mg/kg; from about 5 mg/kg to about 600 mg/kg; from about 6 mg/kg to about 550 mg/kg; from about 7 mg/kg to about 500 mg/kg; from about 8 mg/kg to about 450 mg/kg; from about 9 mg/kg to about 400 mg/kg; from about 5 mg/kg to about 200 mg/kg; from about 2 mg/kg to about 150 mg/kg; from about 5 mg/kg to about 100 mg/kg; from about 10 mg/kg to about 100 mg/kg; and from about 10 mg/kg to about 60 mg/kg.

The kit may comprise a number of containers suitable for a single dose of the one or more checkpoint inhibitors, or a number of such containers suitable for repetitive doses of the one or more checkpoint inhibitors, such as a series of doses, e.g. for use over the course of several days, weeks or months.

In one embodiment, the kit further comprises instructions for administering the checkpoint inhibitor(s), e.g. instructions for administering them via injection, e.g. subcutaneous, intravenous, intra-arterial, intratumoral, intralymphatic, intraperitoneal, intracardiac, intrathecal, or intramuscular injection, or infusion injection.

### DESCRIPTION OF THE DRAWINGS

FIG. 1—FIG. 1a and FIG. 1b illustrate embodiments of anticancer vaccines for use in the method of the invention described as polypeptides having N-terminal starts and C-terminal ends.

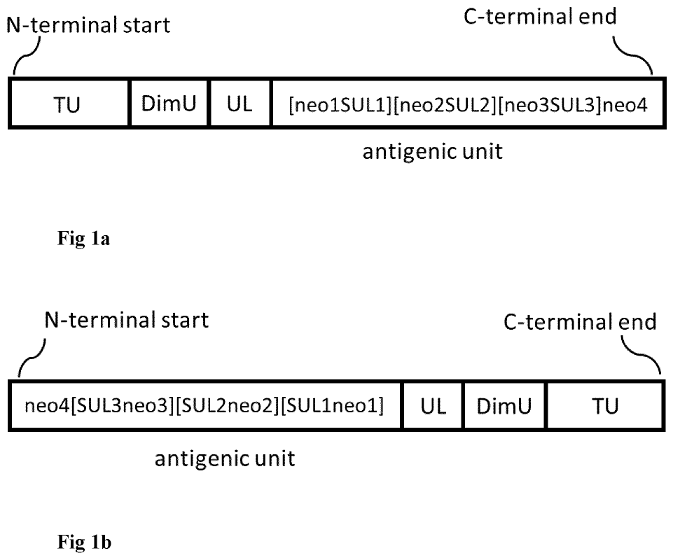

FIG. 2 illustrates the results of Example 2, i.e. the synergistic effect of administering to mice, challenged with CT26 tumor cells, a combined treatment of the checkpoint inhibitor(s) anti-PD-L1 antibody, anti-TIGIT-antibody or anti-PD-L1 and anti-TIGIT-antibody and the anticancer vaccine VB4085. Treatment efficacy (measured as tumor volume) of the combined treatments was compared to treatment efficacy of the monotherapy, i.e. anticancer vaccine VB4085 or anti-PD-L1 antibody or anti-TIGIT-antibody or anti-PD-L1 and anti-TIGIT-antibody and to the negative control, VB1026.

FIG. 3 illustrates the results of Example 2, i.e. the synergistic effect of administering to mice, challenged with CT26 tumor cells, a combined treatment of the checkpoint inhibitor(s) anti-PD-L1 antibody, anti-TIGIT-antibody or anti-PD-L1 and anti-TIGIT-antibody and the anticancer vaccine VB4085. Treatment efficacy (measured as survival % days after tumor challenge) of the combined treatments was compared to treatment efficacy of the monotherapy, i.e. anticancer vaccine VB4085 or anti-PD-L1 antibody or anti-TIGIT-antibody or anti-PD-L1 and anti-TIGIT-antibody and to the negative control, VB1026.

FIG. 4 illustrates the results of Example 3, i.e. the synergistic effect of administering to mice, challenged with CT26 tumor cells, a combined treatment of the checkpoint inhibitor anti-CTLA-4 antibody and the anticancer vaccine VB4085. Treatment efficacy (measured as tumor volume) of the combined treatment was compared to treatment efficacy of the monotherapy, i.e. anticancer vaccine VB4085 or anti-CTLA-4 antibody and to the negative control, VB1026.

FIG. 5 illustrates the results of Example 3, i.e. the synergistic effect of administering to mice, challenged with CT26 tumor cells, a combined treatment of the checkpoint inhibitor anti-CTLA-4 antibody and the anticancer vaccine VB4085. Treatment efficacy (measured as survival % days after tumor challenge) of the combined treatment was compared to treatment efficacy of the monotherapy, i.e. anticancer vaccine VB4085 or anti-CTLA-4 antibody and to the negative control, VB1026.

### EXAMPLES

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims

### Example 1: Design of Anticancer Vaccines

Two DNA constructs were designed comprising nucleotide sequences encoding the following units/elements:

| | | | Sequence: |
|---|---|---|---|
| Unit/Element | Construct VB4085 | Construct VB1026 | SEQ ID NO: 1 |
| Signal | Native leader | Native leader | Amino acids |
| peptide | sequence for | sequence for | 1-23 |
| | human MIP-1α | human MIP-1α | |
| Targeting | Full length human | Full length human | Amino acids |
| unit | MIP-1α sequence | MIP-1α sequence | 24-93 |
| Dimerization | Human hinge- | Human hinge- | Amino acids |
| unit | region 1 from IgG | region 1 from IgG3 | 94-105 |
| | Human hinge- | Human hinge- | Amino acids |
| | region 4 from IgG3 | region 4 from IgG3 | 106-120 |
| | Glycine-serine- | Glycine-serine- | Amino acids |
| | linker | linker | 121-130 |
| | Human CH3 | Human CH3 | Amino acids |
| | domain from IgG3 | domain from IgG3 | 131-237 |
| Unit | Glycine-leucine | Absent | Amino acids |
| linker | linker | | 238-242 |
| Antigenic | See below | Absent | Amino acids |
| unit | | | 243-972 |

### Anticancer Vaccine VB4085:

Previously described exome sequencing and RNA sequencing of the mouse colon cancer cell line CT26 revealed hundreds to thousands of tumor-specific non-synonymous mutations. In silico-based methods were used to identify potential immunogenic epitopes, i.e. epitopes containing a mutation, and 20 of them (Table 1) were chosen for inclusion into the antigenic unit of VB4085. Each of the epitopes consists of 27 amino acids and the epitopes are separated from each other by linkers.

Thus, all epitopes but the terminal epitope were arranged in subunits, each subunit consisting of one epitope and one flexible glycine-serine linker (GGGGSGGGGS (SEQ ID NO: 29)). VB4085 is a model of (a) an individualized DNA anticancer vaccine comprising several patient-specific epitopes, e.g. several neoepitopes and/or several patient-present shared cancer epitopes (with the patient-present shared cancer antigens being mutated patient-present shared cancer antigens) and (b) an off-the-shelf DNA anticancer vaccine comprising several shared cancer epitopes (with the shared cancer antigens being mutated shared cancer antigens).

VB4085 consists of a DNA sequence encoding the polypeptide with amino acid sequence of SEQ ID NO: 1.

TABLE 1

| | Neo- | | SEQ ID |
|---|---|---|---|
| No. | antigen | Sequence | NO: |
| 1 | pepM135 | STMLYIRALKNPSLYGFSSGLNKDGIE | 122 |
| 2 | pepM79 | GALKKLIYAAKLNTSLKALEGERNQVY | 123 |
| 3 | pepM106 | DLVCDFQSFKYYAHATSLAGHLVSCPL | 124 |
| 4 | pepM55 | EKLRNPCPNKEKAYQPPFAFRHVLKLT | 125 |
| 5 | pepM06 | DTLSAMSNPRAMQVLLQIQQGLQTLAT | 126 |
| 6 | pepM69 | GDVKIHAHKVVLANISPYFKAMFTGNL | 127 |
| 7 | pepM08 | EVIQTSKYYMRDVIAIESAWLLELAPH | 128 |
| 8 | pepM152 | FFSFFTHRFGHHVSPQVHIILANLYLL | 129 |
| 9 | pepM01 | VILPQAPSGPSYATYLQPAQAQMLTPP | 130 |
| 10 | pepM171 | LWVYLRPVPRPATIYLQILRLKPLTGE | 131 |
| 11 | pepM149 | FVSPMAHYVPGIMAIESVVARFQFIVP | 132 |
| 12 | pepM89 | TLAFLVLSTPAMFNRALKPFLKSCHFQ | 133 |
| 13 | pepM172 | FLERPMDMPYMIFYPNNPLMTGQLLGS | 134 |
| 14 | pepM176 | IPREVGDGTRVDPFPPVQTWMRLPKLV | 135 |
| 15 | pepM29 | GSLFGSSRVQYVVNPAVKIVFLNIDPS | 136 |
| 16 | pepM174 | NNLQKYIEIYVQKINPSRLPVVIGGLL | 137 |
| 17 | pepM31 | AEYGDYQPEVHGVPYFRLEHYLPARVM | 138 |
| 18 | pepM175 | TPLRKHTVHAIRKFYLEFKGSSPPPRL | 139 |
| 19 | pepM173 | KIYEFDYHLYGQNITMIMTSVSGHLLA | 140 |
| 20 | pepM43 | KSWIHCWKYLSVQSQLFRGSSLLFRRV | 141 |

### Negative Control VB1026:

This construct is identical to VB4085, but comprises neither a unit linker, nor an antigenic unit. It serves as a negative control. VB1026 consists of a DNA sequence encoding the polypeptide with amino acid sequence 1-237 of SEQ ID NO: 1.

All epitope gene sequences were ordered from Genescript (New Jersey, US) and cloned into the expression vector pUMVC4a comprising a nucleotide sequence for the targeting unit and dimerization unit described above.

### Example 2: Embodiment of a Method of Treatment with Anticancer Vaccine and Checkpoint Inhibitor Anti-TIGIT and/or Anti-PD-L1 According to the Invention in CT26-Tumor Challenged Mice Compared to Treatment with Anticancer Vaccine or Checkpoint Inhibitor(s) Alone

The treatment schedule for this study is shown in Table 2 below:

TABLE 2

| | | D 0 | D 7 | D 10 | D 13 | D 17 | D 24 | D 27 | D 31 |
|---|---|---|---|---|---|---|---|---|---|
| | Tumor challenge with CT26 | ↓ | | | | | | | |
| A | VB1026 | x | x | | x | | | x | |
| B | VB4085 | x | x | | x | | | x | |
| C | anti-TIGIT | | | • | | • | • | | • |
| D | anti-PD-L1 | | | • | | • | • | | • |
| E | anti-TIGIT + anti-PD-L1 | | | • | | • | • | | • |
| F | VB4085 + anti-TIGIT | x | x | • | x | • | • | x | • |
| G | VB4085 + anti-PD-L1 | x | x | • | x | • | • | x | • |
| H | VB4085 + anti-TIGIT + anti-PD-L1 | x | x | • | x | • | • | x | • |

x denotes the administration of VB1026 and VB4085, respectively;
• denotes the administration of the checkpoint inhibitor(s).

Anti-PD-L1 antibody (in this Example section also called anti-PD-L1) was purchased from Bio X Cell, USA. This monoclonal antibody reacts with murine PD-L1 (anti-mouse PD-L1 antibody). Anti-TIGIT antibody (in this Example section also called anti-TIGIT) was purchased from Absolute Antibody, USA. This monoclonal antibody reacts with murine TIGIT (anti-mouse TIGIT antibody).

### Treatment of Balb/c Mice:

Each of the groups A-H contained 10 mice, which were inoculated with CT26 tumor cells on day (D) 0 by injection of 5×10⁴ tumor cells in the left leg. On days 0, 7, 13, and 27 the mice were vaccinated intramuscularly with a total of 50 μg of construct VB4085 or of the negative control construct VB1026, 25 μg into each tibial anterior muscle, followed by electroporation. Administration of anti-TIGIT and/or anti-PD-L1 antibody was carried out on days 10, 17, 24, and 31 by intraperitoneal injection of 200 μg of each checkpoint inhibitor, i.e. 200 μg of either anti-TIGIT or anti-PD-L1 antibody or 200 μg of each, anti-PD-L1 antibody and anti-TIGIT antibody, for groups E and H). Tumor size was measured using a caliper. The tumors were measured in two dimensions, length and width, and the height was set equal to the width. The tumor volume was calculated by the formula: Tumor vol.=Length (mm)×width (mm)×height (mm)/2000. The treatment was concluded on day 43.

### Treatment Results:

Statistical significance was determined by two-way ANOVA statistical testing with Tukey's multiple comparison test.

The tumors in the group that had been treated with VB4085 anticancer vaccine alone (group B) appeared to grow slower compared to the VB1026 negative control group (group A). However due to intragroup variation the difference in tumor growth was not statistically different between these two groups.

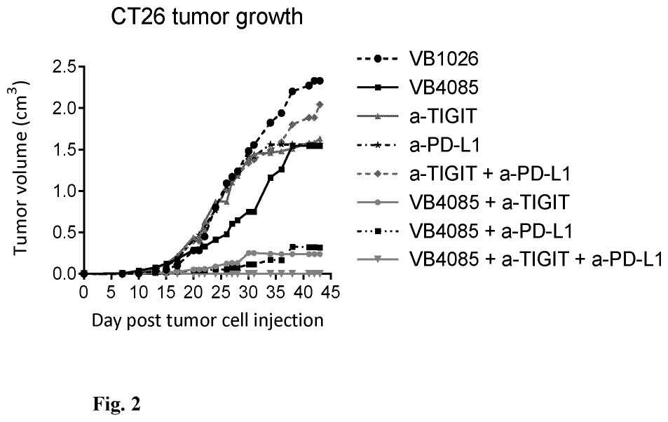

The difference in treatment efficacy (tumor growth) between the groups treated according to an embodiment of the method of the invention (groups F-H) and the VB1026 negative control group was statistically significant (F: p=0.01; G: p=0.005; H: p=0.003, with p being p-value) from day 26 and throughout the experiment (FIG. 2).

The difference in treatment efficacy (tumor growth) between group F receiving VB4085 anticancer vaccine in combination with anti-TIGIT antibody (embodiment of the method of the invention) and the VB4085 anticancer vaccine monotherapy group B was statistically significant (p=0.0004) from day 34 and onwards (FIG. 2).

The difference in treatment efficacy (tumor growth) between group G receiving VB4085 anticancer vaccine in combination with anti-PD-L1 antibody (embodiment of the method of the invention) and the VB4085 anticancer vaccine monotherapy group B was statistically significant (p=0.047) from day 28 and onwards (FIG. 2).

The difference in treatment efficacy (tumor growth) between the group H receiving VB4085 anticancer vaccine in combination with anti-TIGIT antibody and anti-PD-L1 antibody (embodiment of the method of the invention) and the VB4085 monotherapy group B was statistically significant (p=0.032) from day 27 and onwards (FIG. 2).

Treatment efficacy (determined by tumor growth) in the groups treated according to an embodiment of the method of the invention (groups F-H) was in each group significantly better, compared to their respective antibody monotherapy group (groups C-E) from day 26 (FIG. 2).

The tumors in mice of group H (embodiment of the method of the invention) never even grew to a large size and some of the tumor takes were only observed at a single time point.

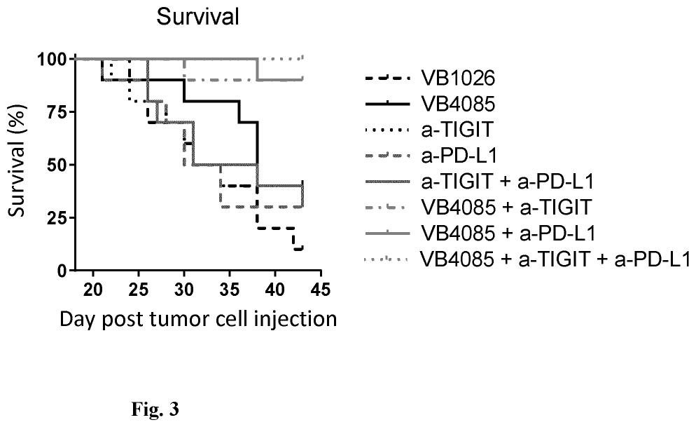

Moreover, the survival of mice (measured as the number of deaths/number of mice per group) treated according to an embodiment of the method of the invention (groups F-H) was markedly better compared to those that had received a treatment with the anticancer vaccine or the checkpoint inhibitor(s) alone (groups B-E), as shown in Table 3 below and in FIG. 3.

TABLE 3

| | | Number of deaths |
|---|---|---|
| A | VB1026 | 9/10 |
| B | VB4085 | 6/10 |
| C | anti-TIGIT | 6/10 |
| D | anti-PD-L1 | 7/10 |
| E | anti-TIGIT + anti-PD-L1 | 7/10 |
| F | VB4085 + anti-TIGIT | 1/10 |
| G | VB4085 + anti-PD-L1 | 1/10 |
| H | VB4085 + anti-TIGIT + anti-PD-L1 | 0/10 |

### Conclusion:

Tumor growth in mice vaccinated with VB4085 anticancer vaccine as a monotherapy (group B) was on average slower compared to tumor growth in the negative control group treated with VB1026 (group A). This is in accordance with previous CT26 tumor challenge studies with mice vaccinated with VB4085 and demonstrates that this DNA anticancer vaccine elicits an immune response against CT26 tumor cells that results in reduced tumor growth.

Others have shown that PD-L1 is expressed by CT26 cells and that the expression is increased in response to IFN-γ exposure, suggesting that the CT26 cells may evade immune surveillance through the PD-1/PD-L1 axis (Lau et al., Nat. Commun 8, 2017, 1-11). The results of the present study showed that blocking PD-L1 by administration of anti-PD-L1 antibodies in combination with the anticancer vaccine VB4085 resulted in an increased anti-tumor efficacy with further reduced tumor growth compared to the VB4085 anticancer vaccine monotherapy.

Previously, it was shown that treating mice inoculated with CT26 tumor cells with anti-TIGIT antibody reduces tumor growth (Johnston et al., Cancer Cell 26(6), 2014, 923-937). The results of the present study showed that blocking the TIGIT signaling pathways by administration of anti-TIGIT antibodies in combination with the anticancer vaccine VB4085 reduced the tumor growth to a greater extent than the VB4085 anticancer vaccine monotherapy.

Combined targeting of TIGIT and PD-1/PD-L1 has been shown to be beneficial compared to targeting either checkpoint protein individually in a CT26 tumor mice model (Johnston et al., Cancer Cell 26(6), 2014, 923-937). These finding demonstrates that the mouse model evades immune surveillance through multiple pathways. In the present study, we have shown that when using a combination of anti-TIGIT and anti-PD-L1 antibodies, the inclusion of the anticancer vaccine VB4085 into such treatment can clearly enhance the immune response against the tumor cells. Such combined treatment according an embodiment of the method of the invention has shown a better treatment efficacy (as determined by tumor growth and survival) compared to any of the three treatments alone.

### Example 3: Embodiment of a Method of Treatment with Anticancer Vaccine and Checkpoint Inhibitor Anti-CTLA-4 According to the Invention in CT26-Tumor Challenged Mice Compared to Treatment with Anticancer Vaccine or Checkpoint Inhibitor Anti-CTLA-4 Alone

Constructs VB4085 and VB1026 as described in Example 1 were used in this study. The treatment schedule for this study is shown in Table 4 below.

TABLE 4

| | | D0 | D8 | D11 | D14 | D18 | D26 | D28 | D32 |
|---|---|---|---|---|---|---|---|---|---|
| | Tumor challenge with CT26 | ↓ | | | | | | | |
| A | VB1026 | x | x | | x | | | x | |
| B | VB4085 | x | x | | x | | | x | |
| C | anti-CTLA-4 | | | • | | • | • | | • |
| D | VB4085 + anti-CTLA-4 | x | x | • | x | • | • | x | • |

x denotes the administration of VB1026 and VB4085, respectively;
• denotes the administration of the checkpoint inhibitor.

Anti-CTLA-4 (CD152) antibody (in this Example section also called anti-CTLA-4) was purchased from Bio X Cell, USA. This monoclonal antibody reacts with murine CTLA-4 (anti-mouse CTLA-4 antibody).

### Treatment of Balb/c Mice:

Each of the groups A-D contained 10 mice, which were inoculated with CT26 tumor cells on day (D) 0 by subcutaneous injection of 5×10⁴ tumor cells in the left thigh. On days 0, 8, 14, and boost day 28 the mice were vaccinated intramuscularly with a total of 50 μg (25 μg for boost) of construct VB4085 or of the negative control construct VB1026, 25 μg into each tibial anterior muscle (for boost: 25 μg into the right tibial anterior muscle), followed by electroporation. Administration of anti-CTLA-4 antibody was carried out on days 11, 18, 26, and 32 by intraperitoneal injection of 200 μg/mice. Tumor size was measured using a caliper. The tumors were measured in two dimensions, length and width, and the height was set equal to the width. The tumor volume was calculated by the formula: Tumor vol.=Length (mm)×width (mm)×height (mm)/2000. The experiment was concluded on day 39.

### Treatment Results:

Statistical significance was determined by two-way ANOVA statistical testing with Tukey's multiple comparison test.

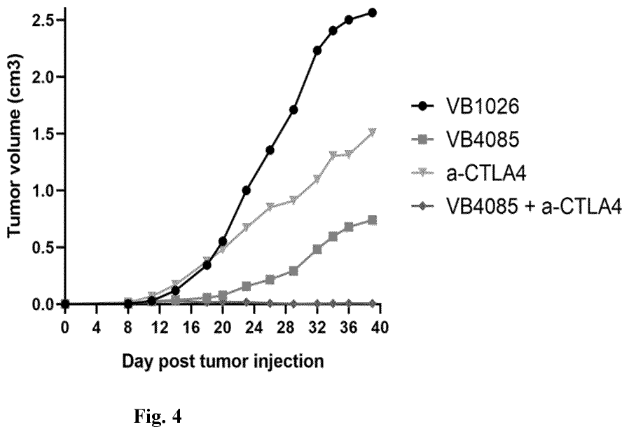

The difference in treatment efficacy (tumor growth) between VB4085 anticancer vaccine monotherapy (group B) compared to the VB1026 negative control group (group A) was statistically significant (p=0.0067) from day 26 and onwards (FIG. 4).

The difference in treatment efficacy (tumor growth) between anti-CTLA-4 antibody monotherapy (group C) compared to the VB1026 negative control group (group A) was statistically significant (p=0.038) from day 32 an onwards (FIG. 4).

The difference in treatment efficacy (tumor growth) between group D receiving VB4085 anticancer vaccine in combination with anti-CTLA-4 antibody (embodiment of method of the invention) and the VB1026 negative control group (group A) was statistically significant (p=0.025) from day 23 and onwards (FIG. 4).

The difference in treatment efficacy (tumor growth) between the group treated according to an embodiment of the method of the invention (group D) and the VB4085 anticancer vaccine monotherapy group B was not statistically significant (p >0.05) (FIG. 4).

The difference in treatment efficacy (tumor growth) between the group D receiving VB4085 anticancer vaccine in combination with anti-CTLA-4 antibody (embodiment of the method of the invention) and the anti-CTLA4 antibody monotherapy group C was statistically significant (p=0.032) from day 32 and onwards (FIG. 4).

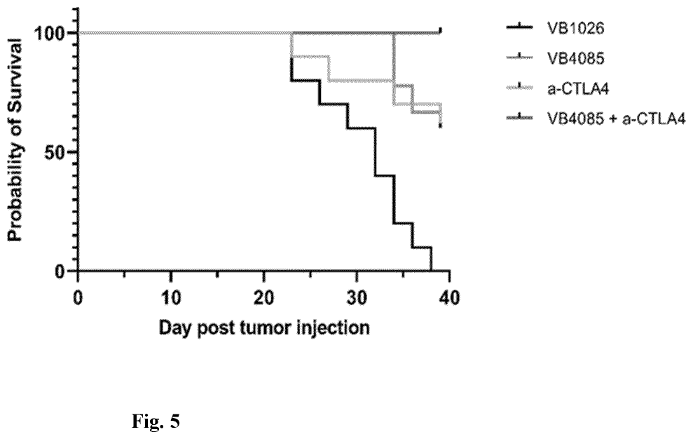

Moreover, the survival of mice (measured as the number of deaths/number of mice per group) treated according to an embodiment of the method of the invention (groups D) was markedly better compared to those that had received a treatment with the anticancer vaccine or the checkpoint inhibitor alone (groups B and C), as shown in Table 5 below and in FIG. 5.

TABLE 5

| | | Number of deaths |
|---|---|---|
| A | VB1026 | 10/10 |
| B | VB4085 | 3/9* |
| C | anti-CTLA-4 | 6/10 |
| D | VB4085 + anti-CTLA-4 | 0/10 |

*One animal in the VB4085 group developed spontaneous rectal prolapse on day 32 and had to be euthanized. The animal had no tumor on day 32.

### Conclusion:

Tumor growth in mice vaccinated with VB4085 anticancer vaccine as a monotherapy (group B) had a significantly slower tumor growth compared to the negative control group treated with VB1026 (group A). This is in accordance with previous CT26 tumor challenge studies with mice vaccinated with VB4085 and demonstrates that this DNA anticancer vaccine elicits an immune response against CT26 tumor cells that results in reduced tumor growth.

From previous reports, others have shown anti-cancer efficacy in CT26 tumor challenged mice treated with anti-CTLA-4 antibody monotherapy (Pedersen et al., Cancer Lett 235(2), 2006, 229-238, reviewed in Grosso et al, Cancer Immun 13(1), 2013, 5).

The results of the present study show that blocking the CTLA-4 signaling pathways by administration of anti-CTLA4 antibodies as monotherapy cause a partial anti-tumor response, whereas a clearly enhanced immune response against the tumor cells were observed in mice treated with anti-CTLA4 antibodies in combination with the VB4085 anticancer vaccine, where 9 out of 10 mice were tumor free at the end of study (day 39).

Such combined treatment according to an embodiment of the method of the invention showed a better treatment efficacy (as determined by tumor growth and survival) compared to the two different monotherapies.

### Example 4: Embodiment of a Method of Treatment According to the Invention Comprising Administration of an Individualized DNA Anticancer Vaccine Comprising VB10.NEO in Combination with Atezolizumab

Adult patients are treated are treated with an individualized DNA anticancer vaccine comprising VB10.NEO in combination with atezolizumab. The patients are those with locally advanced and metastatic tumors that have progressed after at least 1 available standard therapy; or for whom standard therapy has proven to be ineffective or intolerable, or is considered inappropriate; or for whom a clinical trial of an investigational agent is a recognized standard of care

In one embodiment, the patients have at least one of the following tumors: melanoma, NSCLC, RCC, UC, HNSCC, TNBC, gastric/GEJ cancer, cervical, anal, or MSI-high tumors

In one embodiment, VB10.NEO (3 mg in a pharmaceutically acceptable carrier) is administered by intramuscular injection for an induction course Q3W (4 doses) followed by maintenance doses Q6W (6 doses) and Q12W (5 doses). Atezolizumab 1200 mg is administered by intravenous infusion on day 1 of 21 day cycles.

In another embodiment, VB10.NEO (6 mg in a pharmaceutically acceptable carrier) is administered by intramuscular injection for an induction course Q3W (4 doses) followed by maintenance doses Q6W (6 doses) and Q12W (5 doses). Atezolizumab 1200 mg is administered by intravenous infusion on day 1 of 21 day cycles.

During and after treatment (from baseline and up to 27 months), primary outcome measures are the following:
Incidence and severity of adverse events
Changes for measurements done prior and after the first VB10.NEO injection of each cycle for the following vital signs: systolic blood pressure (mmHg); diastolic blood pressure (mmHg); pulse rate (bpm); respiration rate (breaths/min); body temperature (° C.)
Changes for clinical laboratory parameters analyzed locally prior and after the first VB10.NEO injection of each cycle, including hematology, chemistry panel, coagulation, thyroid function testing, C-reactive protein, urinalysis and serology

During and after treatment, secondary outcome measures are the following:
Assessment of the antigen-specific immune response elicited by VB10.NEO administered in combination with atezolizumab (from baseline and up to 25 months): number and magnitude of antigen-specific T cell responses before and after initiation of treatment;
Objective response rate (from baseline and up to 27 months): determination of complete or partial response;
Duration of response (from baseline and up to 27 months): the time from the first occurrence of a documented objective response to disease progression or death from any cause (whichever occurs first);
Progression free survival (from baseline and up to 27 months): the time from the first treatment to the first occurrence of disease progression or death from any cause (whichever occurs first);
Overall survival (from baseline and up to 27 months): the time from the first treatment to death from any cause;
Determining serum concentration of atezolizumab at specified time points (from baseline and up to 25 months);
Prevalence of antidrug antibodies (ADAs) to atezolizumab at baseline and incidence of ADAs to atezolizumab (from baseline and up to 25 months)

In one embodiment, the method of treatment comprises administering to a patient in need thereof an effective amount of an individualized DNA anticancer vaccine comprising VB10.NEO in combination with atezolizumab.

In some embodiments, the patient at least one of the following tumor types: melanoma, NSCLC, RCC, UC, HNSCC, TNBC, gastric/GEJ cancer, cervical, anal, or MSI-high tumors. In some embodiments, the patient has a locally advanced or metastatic tumor that has progressed after at least 1 available standard therapy; and/or for whom standard therapy has proven to be ineffective or intolerable, or is considered inappropriate; and/or or for whom a clinical trial of an investigational agent is a recognized standard of care

In some embodiments, the effective amount is VB10.NEO (3 mg in a pharmaceutically acceptable carrier) administered by intramuscular injection for an induction course Q3W (4 doses) followed by maintenance doses Q6W (6 doses) and Q12W (5 doses) and atezolizumab 1200 mg administered by intravenous infusion on day 1 of 21 day cycles. In some embodiments, VB10.NEO (6 mg in a pharmaceutically acceptable carrier) is administered by intramuscular injection for an induction course Q3W (4 doses) followed by maintenance doses Q6W (6 doses) and Q12W (5 doses) and tezolizumab 1200 mg is administered by intravenous infusion on day 1 of 21 day cycles.

In some embodiments, the method of treatment results in an improved overall response rate (ORR) and/or an improved duration of response (DOR) and/or an improved progression free survival (PFS) and/or an improved overall survival (OS) compared to a reference population. In some embodiments the reference population is one treated with a Standard of Care (SOC) for the tumor.

Sequences

SEQ ID NO: 1
VB4085 (amino acids 1-972)/VB1026 (amino acids 1-237).
M¹QVSTAALAVLLCTMALCNQVLS²³A²⁴PLAADTPTACCFSYTSRQIPQNFIAD
YFETSSQCSKPSVIFLTKRGRQVCADPSEEWVQKYVSDLELSA⁹³E⁹⁴LKTPLGDT
THT¹⁰⁵E¹⁰⁶PKSCDTPPPCPRCP¹²⁰G¹²¹GGSSGGGSG¹³⁰G¹³¹QPREPQVYTLPPSREE
MTKNQVSLTCLVKGFYPSDIAVEWESSGQPENNYNTTPPMLDSDGSFFLYSKL
TVDKSRWQQGNIFSCSVMHEALHNRFTQKSLSLSPGK²³⁷G²³⁸LGGL²⁴²STMLYI
RALKNPSLYGFSSGLNKDGIEGGGGSGGGGSGALKKLIYAAKLNTSLKALEGE
RNQVYGGGGSGGGGSDLVCDFQSFKYYAHATSLAGHLVSCPLGGGGSGGGG
SEKLRNPCPNKEKAYQPPFAFRHVLKLTGGGGSGGGGSDTLSAMSNPRAMQV
LLQIQQGLQTLATGGGGSGGGGSGDVKIHAHKVVLANISPYFKAMFTGNLGG
GGSGGGGSEVIQTSKYYMRDVIAIESAWLLELAPHGGGGSGGGGSFFSFFTHR
FGHHVSPQVHIILANLYLLGGGGSGGGGSVILPQAPSGPSYATYLQPAQAQML
TPPGGGGSGGGGSLWVYLRPVPRPATIYLQILRLKPLTGEGGGGSGGGGSFVS
PMAHYVPGIMAIESVVARFQFIVPGGGGSGGGGSTLAFLVLSTPAMFNRALKP
FLKSCHFQGGGGSGGGGSFLERPMDMPYMIFYPNNPLMTGQLLGSGGGGSGG
GGSIPREVGDGTRVDPFPPVQTWMRLPKLVGGGGSGGGGSGSLFGSSRVQYV
VNPAVKIVFLNIDPSGGGGSGGGGSNNLQKYIEIYVQKINPSRLPVVIGGLLGG
GGSGGGGSAEYGDYQPEVHGVPYFRLEHYLPARVMGGGGSGGGGSTPLRKH
TVHAIRKFYLEFKGSSPPPRLGGGGSGGGGSKIYEFDYHLYGQNITMIMTSVSG
HLLAGGGGSGGGGSKSWIHCWKYLSVQSQLFRGSSLLFRRV
SEQ ID NO: 2
Signal peptide
MNFGLRLIFLVLTLKGVQC
SEQ ID NO: 3
Signal peptide
MDAMKRGLCCVLLLCGAVFVSP
SEQ ID NO: 4
Nucleotide sequence encoding amino acids 1-23 of SEQ ID NO: 1
ATGCAGGTCTCCACTGCTGCCCTTGCCGTCCTCCTCTGCACCATGGCTCTCT
GCAACCAGGTCCTCTCT
SEQ ID NO 5:
Nucleotide sequence encoding amino acids 24-93 of SEQ ID NO: 1
GCACCACTTGCTGCTGACACGCCGACCGCCTGCTGCTTCAGCTACACCTCC
CGACAGATTCCACAGAATTTCATAGCTGACTACTTTGAGACGAGCAGCCAG
TGCTCCAAGCCCAGTGTCATCTTCCTAACCAAGAGAGGCCGGCAGGTCTGT
GCTGACCCCAGTGAGGAGTGGGTCCAGAAATACGTCAGTGACCTGGAGCT
GAGTGCC
SEQ ID NO 6:
Nucleotide sequence encoding amino acids 94-105 of SEQ ID NO: 1
GAGCTCAAAACCCCACTTGGTGACACAACTCACACA
SEQ ID NO 7:
Nucleotide sequence encoding amino acids 106-120 of SEQ ID NO: 1
GAGCCCAAATCTTGTGACACACCTCCCCCGTGCCCAAGGTGCCCA
SEQ ID NO: 8
Nucleotide sequence encoding amino acids 94-120 of SEQ ID NO: 1
GAGCTCAAAACCCCACTTGGTGACACAACTCACACAGAGCCCAAATCTTGT
GACACACCTCCCCCGTGCCCAAGGTGCCCA
SEQ ID NO 9:
Nucleotide sequence encoding amino acids 121-130 of SEQ ID NO: 1
GGCGGTGGAAGCAGCGGAGGTGGAAGTGGA
SEQ ID NO 10:
Nucleotide sequence encoding amino acids 131-237 of SEQ ID NO: 1
GGACAGCCCCGAGAACCACAGGTGTACACCCTGCCCCCATCCCGGGAGGA
GATGACCAAGAACCAGGTCAGCCTGACCTGCCTGGTCAAAGGCTTCTACCC
CAGCGACATCGCCGTGGAGTGGGAGAGCAGCGGGCAGCCGGAGAACAACT
ACAACACCACGCCTCCCATGCTGGACTCCGACGGCTCCTTCTTCCTCTACA
GCAAGCTCACCGTGGACAAGAGCAGGTGGCAGCAGGGGAACATCTTCTCA
TGCTCCGTGATGCATGAGGCTCTGCACAACCGCTTCACGCAGAAGAGCCTC
TCCCTGTCTCCGGGTAAA
SEQ ID NO 11:
Nucleotide sequence encoding amino acids 238-242 of SEQ ID NO: 1
GGCCTCGGTGGCCTG
SEQ ID NO 12:
Nucleotide sequence encoding amino acids 94-237 of SEQ ID NO: 1
GAGCTCAAAACCCCACTTGGTGACACAACTCACACAGAGCCCAAATCTTGT
GACACACCTCCCCCGTGCCCAAGGTGCCCAGGCGGTGGAAGCAGCGGAGG
TGGAAGTGGAGGACAGCCCCGAGAACCACAGGTGTACACCCTGCCCCCAT
CCCGGGAGGAGATGACCAAGAACCAGGTCAGCCTGACCTGCCTGGTCAAA
GGCTTCTACCCCAGCGACATCGCCGTGGAGTGGGAGAGCAGCGGGCAGCC
GGAGAACAACTACAACACCACGCCTCCCATGCTGGACTCCGACGGCTCCTT
CTTCCTCTACAGCAAGCTCACCGTGGACAAGAGCAGGTGGCAGCAGGGGA
ACATCTTCTCATGCTCCGTGATGCATGAGGCTCTGCACAACCGCTTCACGC
AGAAGAGCCTCTCCCTGTCTCCGGGTAAA
SEQ ID NO 13:
Heavy chain amino acid sequence of atezolizumab
EVQLVESGGGLVQPGGSLRLSCAASGFTFSDSWIHWVRQAPGKGLEWVAWIS
PYGGSTYYADSVKGRFTISADTSKNTAYLQMNSLRAEDTAVYYCARRHWPG
GFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVV
VDVSHEDPEVKFNWYVDGVEVHNAKTKPREEQYASTYRVVSVLTVLHQDWL
NGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQ
GNVFSCSVMHEALHNHYTQKSLSLSPG
SEQ ID NO 14:
Light chain amino acid sequence of atezolizumab
DIQMTQSPSSLSASVGDRVTITCRASQDVSTAVAWYQQKPGKAPKLLIYSASF
LYSGVPSRFSGSGSGTDFTLTISSLQPEDFATYYCQQYLYHPATFGQGTKVEIK
RTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGE
C
SEQ ID NO 15:
HVR-H1 of atezolizumab
GFTFSDSWIH
SEQ ID NO 16:
HVR-H21 of atezolizumab
AWISPYGGSTYYADSVKG
SEQ ID NO 17:
HVR-H3 of atezolizumab
RHWPGGFDY
SEQ ID NO 18:
HVR-L1 of atezolizumab
RASQDVSTAVA
SEQ ID NO 19:
HVR-L2 of atezolizumab
SASFLYS
SEQ ID NO 20:
HVR-L3 of atezolizumab
QQYLYHPAT
SEQ ID NO 21:
HV of atezolizumab
EVQLVESGGGLVQPGGSLRLSCAASGFTFSDSWIHWVRQAPGKGLEWVAWIS
PYGGSTYYADSVKGRFTISADTSKNTAYLQMNSLRAEDTAVYYCARRHWPG
GFDYWGQGTLVTVSS
SEQ ID NO: 22
HL of atezolizumab
DIQMTQSPSSLSASVGDRVTITCRASQDVSTAVAWYQQKPGKAPKLLIYSASF
LYSGVPSRFSGSGSGTDFTLTISSLQPEDFATYYCQQYLYHPATFGQGTKVEIK
R
SEQ ID NO: 23
Nucleic acid sequence of VB10.NEO
ATGCAGGTCTCCACTGCTGCCCTTGCCGTCCTCCTCTGCACCATGGCTCTCT
GCAACCAGGTCCTCTCTGCACCACTTGCTGCTGACACGCCGACCGCCTGCT
GCTTCAGCTACACCTCCCGACAGATTCCACAGAATTTCATAGCTGACTACT
TTGAGACGAGCAGCCAGTGCTCCAAGCCCAGTGTCATCTTCCTAACCAAGA
GAGGCCGGCAGGTCTGTGCTGACCCCAGTGAGGAGTGGGTCCAGAAATAC
GTCAGTGACCTGGAGCTGAGTGCCGAGCTCAAAACCCCACTTGGTGACAC
AACTCACACAGAGCCCAAATCTTGTGACACACCTCCCCCGTGCCCAAGGTG
CCCAGGCGGTGGAAGCAGCGGAGGTGGAAGTGGAGGACAGCCCCGAGAA
CCACAGGTGTACACCCTGCCCCCATCCCGGGAGGAGATGACCAAGAACCA
GGTCAGCCTGACCTGCCTGGTCAAAGGCTTCTACCCCAGCGACATCGCCGT
GGAGTGGGAGAGCAGCGGGCAGCCGGAGAACAACTACAACACCACGCCTC
CCATGCTGGACTCCGACGGCTCCTTCTTCCTCTACAGCAAGCTCACCGTGG
ACAAGAGCAGGTGGCAGCAGGGGAACATCTTCTCATGCTCCGTGATGCAT
GAGGCTCTGCACAACCGCTTCACGCAGAAGAGCCTCTCCCTGTCTCCGGGT
AAAGGCCTCGGTGGCCTG
SEQ ID NO: 113
Nucleic acid sequence of tetramerization unit derived from p53
AAGCCTCTGGACGGAGAGTATTTCACTCTCCAGATCCGGGGCCCCGAAAG
GTTCGAAATGTTCCGGGAGCTTAACGAGGCCTTGGAGCTGAAAGACGCAC
AGGCCGGAAAGGAACCG
SEQ ID NO: 114
HEYGAEALERAG
SEQ ID NO: 115
Nucleic acid sequence CLEC9 ligand
TGGCCCAGGTTCCACAGCAGCGTGTTCCACACCCAC
SEQ ID NO: 116
Nucleic acid sequence human collagen-derived trimerization domain
GCTGGGCAGGTGAGGATCTGGGCCACATACCAGACCATGCTGGACAAGAT
CCGGGAGGTGCCGGAGGGCTGGCTCATCTTTGTGGCCGAGAGGGAAGAGC
TCTATGTACGCGTTAGAAATGGCTTCCGGAAGGTGCTGCTGGAGGCCCGGA
CAGCCCTCCCGAGAGGCACGGGCAATGAG
SEQ ID NO: 117
Trimerization unit is the C-terminal domain of T4 fibritin
GYIPEAPRDGQAYVRKDGEWVLLSTFL
SEQ ID NO: 119
Nucleic acid sequence of hinge region derived from IgM
GCCGAACTCCCGCCCAAGGTGTCCGTGTTCGTCCCTCCCCGCGATGGGTTC
TTCGGCAATCCACGAAAATCCAAACTGATTTGTCAGGCCACCGGCTTCTCC
CCCCGACAGATCCAGGTGAGTTGGCTACGAGAGGGTAAACAGGTGGGGAG
CGGAGTGACCACTGACCAGGTGCAGGCCGAGGCCAAGGAAAGCGGACCCA
CAACATACAAAGTGACAAGCACTCTGACGATTAAGGAGTCAGACTGGCTC
GGCCAATCCATGTTTACATGCCGGGTTGATCACAGAGGGTTGACCTTCCAA
CAGAACGCATCCAGTATGTGCGTTCCAGAT
SEQ ID NO: 120
Nucleic acid sequence of dHLX protein
GGAGAACTGGAGGAATTACTTAAACATCTCAAGGAGTTGCTCAAAGGCCC
TAGGAAGGGAGAACTGGAGGAACTCCTCAAACATCTCAAGGAGTTACTAA
AGGGA
SEQ ID NO: 121
mCCL19
ATGGCCCCCCGTGTGACCCCACTCCTGGCCTTCAGCCTGCTGGTTCTCTGGA
CCTTCCCAGCCCCAACTCTGGGGGGTGCTAATGATGCGGAAGACTGCTGCC
TGTCTGTGACCCAGCGCCCCATCCCTGGGAACATCGTGAAAGCCTTCCGCT
ACCTTCTTAATGAAGATGGCTGCAGGGTGCCTGCTGTTGTGTTCACCACAC
TAAGGGGCTATCAGCTCTGTGCACCTCCTGACCAGCCCTGGGTGGATCGCA
TCATCCGAAGACTGAAGAAGTCTTCTGCCAAGAACAAAGGCAACAGCACC
AGAAGGAGCCCTGTGTCT

### Embodiments

1. A method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit, such as a dimerization unit, and an antigenic unit comprising one or more cancer antigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

2. The method according to embodiment 1, wherein the anticancer vaccine is an individualized anticancer vaccine.

3. The method according to embodiment 2, wherein the antigenic unit comprises one or more neoantigens or parts thereof.

4. The method according to embodiment 3, wherein the antigenic unit comprises one or more parts of one or more neoantigens.

5. The method according to embodiment 4, wherein said parts are neoepitopes.

6. The method according to embodiment 5, wherein the antigenic unit comprises several neoepitopes, such as several neoepitopes which are separated from each other by linkers.

7. The method according to any of embodiments 5 to 6, wherein the antigenic unit comprises n−1 antigenic subunits, each subunit comprising a neoepitope and a subunit linker, and a terminal neoepitope, and wherein n is the number of neoepitopes in said antigenic unit and n is an integer of from 1 to 50.

8. The method according to any of embodiments 5 to 7, wherein the neoepitopes have a length of from 7 to 30 amino acids such as from 7 to 10 amino acids (such as 7, 8, 9 or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

9. The method according to any of embodiments 3 to 8, wherein the antigenic unit further comprises one or more patient-present shared cancer antigens or parts thereof.

10. The method according to embodiment 9, wherein the antigenic unit further comprises one or more parts of one or more patient-present shared cancer antigens.

11. The method according to embodiment 10, wherein said parts are epitopes.

12. The method according to embodiment 11, wherein the antigenic unit further comprises several epitopes.

13. The method according to any of embodiments 9 to 12, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

14. The method according to embodiment 2, wherein the antigenic unit comprises one or more patient-present shared cancer antigens or parts thereof.

15. The method according to embodiment 14, wherein the antigenic unit comprises one or more parts of one or more patient-present shared cancer antigens.

16. The method according to embodiment 15, wherein said parts are epitopes.

17. The method according to embodiment 16, wherein the antigenic unit comprises several epitopes.

18. The method according to any of embodiments 14 to 17, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

19. The method according to embodiment 1, wherein the anticancer vaccine is a non-individualized anticancer vaccine.

20. The method according to embodiment 19, wherein the antigenic unit comprises one or more shared cancer antigens or parts thereof.

21. The method according to embodiment 20, wherein the antigenic unit comprises one or more parts of one or more shared cancer antigens.

22. The method according to embodiment 21, wherein said parts are epitopes.

23. The method according to embodiment 22, wherein the antigenic unit comprises several epitopes.

24. The method according to any of embodiments 20 to 23, wherein the shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations, scFvs derived from a monoclonal Ig produced by myeloma or lymphoma, telomerase, HIV antigens, tyrosinase, tyrosinase related protein (TRP)-1, TRP-2, melanoma antigen, prostate specific antigen and HPV antigens.

25. The method according to any of embodiments 1 to 24, wherein the antigenic unit comprises up to 3500 amino acids, such as from about 21 to about 2000 amino acids or from about 60 to 3500 amino acids, such as from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

26. The method according to any of embodiments 1 to 25, wherein the targeting unit is or comprises a moiety that interacts with surface molecules on the antigen-presenting cells.

27. The method according to embodiment 26, wherein the surface molecule is selected from the group consisting of HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5. 28. The method according to any of embodiments 26 and 27, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II anti-CD40, anti-TLR-2, anti-TLR-4, anti-TLR-5 or anti-CLEC9A..

29. The method according to embodiment 28, wherein the targeting unit comprises or consists of human MIP-1α (LD78B, CCL3L1).

30. The method according to embodiment 29, wherein the targeting unit comprises an amino acid sequence having at least 80% sequence identity to the amino acid sequence 24-93 of SEQ ID NO: 1, such as comprising the amino acid sequence 26-93 of SEQ ID NO: 1 or comprising the amino acid sequence 28-93 of SEQ ID NO: 1.

31. The method according to embodiment 30, wherein the targeting unit consists of an amino acid sequence having at least 80% sequence identity to the amino acid sequence 24-93 of SEQ ID NO: 1, such as consisting of the amino acid sequence 26-93 of SEQ ID NO: 1 or consisting of the amino acid sequence 28-93 of SEQ ID NO: 1.

32. The method according to embodiment 31, wherein the targeting unit consists of the amino acid sequence 24-93 of SEQ ID NO: 1.

33. The method according to any of embodiments 1 to 32, wherein the multimerization unit, such as a dimerization unit, comprises a hinge region, such as hinge exon h1 and hinge exon h4.

34. The method according to embodiment 33, wherein the hinge region has the ability to form one or more covalent bonds.

35. The method according to any of embodiments 33 to 34, wherein the hinge region is Ig derived.

36. The method according to any of embodiments 33 to 35, wherein the anticancer vaccine comprises a dimerization unit and said dimerization unit further comprises another domain that facilitates dimerization.

37. The method according to embodiment 36, wherein the other domain is an immunoglobulin domain, preferably an immunoglobulin constant domain.

38. The method according to any of embodiments 36 to 37, wherein the other domain is a carboxyterminal C domain derived from IgG, preferably from IgG3.

39. The method according to any of embodiments 36 to 38, wherein the dimerization unit further comprises a dimerization unit linker, such as glycine-serine rich linker, such as GGGSSGGGSG (SEQ ID NO: 118).

40. The method according to embodiment 39, wherein the dimerization unit linker connects the hinge region and the other domain that facilitates dimerization.

41. The method according to any of embodiments 36 to 40, wherein the dimerization unit comprises hinge exon hl and hinge exon h4, a dimerization unit linker and a CH3 domain of human IgG3.

42. The method according to embodiment 41, wherein the dimerization unit comprises an amino acid sequence having at least 80% sequence identity to the amino acid sequence 94-237 of SEQ ID NO: 1.

43. The method according to embodiment 42, wherein the dimerization unit consists of an amino acid sequence having at least 80% sequence identity to the amino acid sequence 94-237 of SEQ ID NO: 1.

44. The method according to embodiment 43, wherein the dimerization unit consists of the amino acid sequence 94-237 of SEQ ID NO: 1.

45. The method according to any of embodiments 1 to 44, wherein the anticancer vaccine further comprises a unit liker that connects the antigenic unit to the multimerization unit, such as dimerization unit, and wherein the unit linker is a non-immunogenic linker and/or flexible or rigid linker.

46. The method according to any of embodiments 1 to 45, wherein the anticancer vaccine is a polynucleotide, preferably an RNA or DNA.

47. The method according to embodiment 46, wherein the polynucleotide further comprises a nucleotide sequence encoding a signal peptide.

48. The method according to embodiment 47, wherein the signal peptide is selected from the list consisting of Ig VH signal peptide, human TPA signal peptide and human MIP-1α signal peptide.

49. The method according to any of embodiments 47 to 48, wherein the signal peptide comprises an amino acid sequence having at least 85% sequence identity to the amino acid sequence 1-23 of SEQ ID NO: 1.

50. The method according to embodiment 49, wherein the signal peptide consists of an amino acid sequence having at least 85% sequence identity to the amino acid sequence 1-23 of SEQ ID NO: 1.

51. The method according to embodiment 50, wherein the signal peptide consists of the amino acid sequence 1-23 of SEQ ID NO: 1.

52. The method according to any of embodiments 46 to 51, wherein the polynucleotide is a DNA.

53. The method according to embodiment 52, wherein the polynucleotide is a DNA, which is comprised in a vector.

54. The method according to any of embodiments 1 to 53, wherein the anticancer vaccine further comprises a pharmaceutically acceptable carrier or diluent.

55. The method according to any of embodiments 1 to 45, wherein the anticancer vaccine is a polypeptide or a multimeric protein, such as a dimeric protein.

56. The method according to embodiment 55, wherein the anticancer vaccine further comprises a pharmaceutically acceptable carrier or diluent.

57. The method according to embodiment 56, wherein the anticancer vaccine further comprises an adjuvant.

58. The method according to any of embodiments 1 to 57 wherein the one or more checkpoint inhibitor is selected from the group consisting of an anti-PD-L1 antibody, an anti-TIGIT antibody, an anti-CTLA-4-antibody and an anti-PD1-antibody, such as wherein the checkpoint inhibitor is an anti-PD-L1 antibody or an anti-TIGIT antibody or an anti-CTLA-4-antibody or an anti-PD1-antibody or wherein the checkpoint inhibitors are an anti-PD-L1 antibody and an anti-TIGIT antibody or an anti-PD-L1 antibody and an anti-CTLA-4-antibody or an anti-PD-1 antibody and an anti-TIGIT antibody or an anti-PD-1 antibody and an anti-CTLA-4-antibody or an anti-TIGIT antibody and an anti-CTLA-4-antibody or an anti-PD-L1 antibody and an anti-PD-1 antibody or wherein the checkpoint inhibitors are an anti-PD-L1 antibody, an anti-PD-1 antibody and an anti-CTLA-4-antibody or an anti-PD-L1 antibody, an anti-PD-1 antibody and an anti-TIGIT antibody or an anti-PD-L1 antibody, an anti-CTLA-4-antibody and an anti-TIGIT antibody or an anti-PD-1 antibody, an anti-CTLA-4-antibody and an anti-TIGIT antibody or wherein the checkpoint inhibitors are anti-PD-LI antibody, anti-TIGIT antibody, anti-CTLA-4-antibody and anti-PD1-antibody.

59. The method according to embodiment 58, wherein the checkpoint inhibitor is an anti-PD-L1 antibody.

60. The method according to embodiment 58, wherein the checkpoint inhibitor is an anti-TIGIT antibody.

61. The method according to embodiment 58, wherein the checkpoint inhibitors are an anti-PD-L1 antibody and an anti-TIGIT antibody.

62. The method according to embodiment 58, wherein the checkpoint inhibitor is an anti-CTLA-4 antibody.

63. The method according to any of embodiments 1 to 62, wherein the one or more checkpoint inhibitor is comprised in a composition suitable for injection, such as infusion injection.

64. The method according to any of embodiments 1 to 63, wherein the one or more checkpoint inhibitors are administered concurrently with the anticancer vaccine.

65. The method according to any of embodiments 1 to 62, wherein the one or more checkpoint inhibitors are administered prior to the first administration of the anticancer vaccine.

66. The method according to any of embodiments 1 to 62, wherein the anticancer vaccine is administered prior to the first administration of the one or more checkpoint inhibitors.

67. The method according to any of embodiments 1 to 66, wherein each of the anticancer vaccine and the one or more checkpoint inhibitors are administered in repetitive doses.

68. The method according to any of embodiments 1 to 67, wherein the cancer is a solid cancer or a liquid cancer.

69. The method according to any of embodiments 1 to 68, wherein the cancer is selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

70. A kit comprising
(a) an anticancer vaccine comprising
(i) a polynucleotide comprising a nucleotide sequence encoding a targeting unit that targets antigen-presenting cells, a multimerization unit, such as a dimerization unit, and an antigenic unit comprising one or more cancer antigens; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

71. The kit according to embodiment 70, wherein the anticancer vaccine is an individualized anticancer vaccine.

72. The kit according to embodiment 71, wherein the antigenic unit comprises one or more neoantigens or parts thereof.

73. The kit according to embodiment 72, wherein the antigenic unit comprises one or more parts of one or more neoantigens.

74. The kit according to embodiment 73, wherein said parts are neoepitopes.

75. The kit according to embodiment 74, wherein the antigenic unit comprises several neoepitopes, such as several neoepitopes which are separated from each other by linkers.

76. The kit according to any of embodiments 74 and 75, wherein the antigenic unit comprises n−1 antigenic subunits, each subunit comprising a neoepitope and a subunit linker, and a terminal neoepitope, and wherein n is the number of neoepitopes in said antigenic unit and n is an integer of from 1 to 50.

77. The kit according to any of embodiments 74 to 76, wherein the neoepitopes have a length of from 7 to 30 amino acids, such as from 7 to 10 amino acids (such as 7, 8, 9 or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

78. The kit according to any of embodiments 72 to 77, wherein the antigenic unit further comprises one or more patient-present shared cancer antigens or parts thereof.

79. The kit according to embodiment 78, wherein the antigenic unit further comprises one or more parts of one or more patient-present shared cancer antigens.

80. The kit according to embodiment 79, wherein said parts are epitopes.

81. The kit according to embodiment 80, wherein the antigenic unit further comprises several epitopes.

82. The kit according to any of embodiments 78 to 81, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

83. The kit according to embodiment 71, wherein the antigenic unit comprises one or more patient-present shared cancer antigens or parts thereof.

84. The kit according to embodiment 83, wherein the antigenic unit comprises one or more parts of one or more patient-present shared cancer antigens.

85. The kit according to embodiment 84, wherein said parts are epitopes.

86. The kit according to embodiment 85, wherein the antigenic unit comprises several epitopes.

87. The kit according to any of embodiments 83 to 86, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

88. The kit according to embodiment 70, wherein the anticancer vaccine is a non-individualized anticancer vaccine.

89. The kit according to embodiment 88, wherein the antigenic unit comprises one or more shared cancer antigens or parts thereof.

90. The kit according to embodiment 89, wherein the antigenic unit comprises one or more parts of one or more shared cancer antigens.

91. The kit according to embodiment 90, wherein said parts are epitopes.

92. The kit according to embodiment 91, wherein the antigenic unit comprises several epitopes.

93. The kit according to any of embodiments 89 to 92, wherein the shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations, scFvs derived from a monoclonal Ig produced by myeloma or lymphoma, telomerase, HIV antigens, tyrosinase, tyrosinase related protein (TRP)-1, TRP-2, melanoma antigen, prostate specific antigen and HPV antigens.

94. The kit according to any of embodiments 70 to 93, wherein the antigenic unit comprises up to 3500 amino acids, such as from about 21 to about 2000 amino acids or from about 60 to 3500 amino acids, such as from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

95. The kit according to any of embodiments 70 to 96, wherein the targeting unit is or comprises a moiety that interacts with surface molecules on the antigen-presenting cells.

96. The kit according to embodiment 95, wherein the surface molecule is selected from the group consisting of HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5.

97. The kit according to any of embodiments 95 and 96, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II anti-CD40, anti-TLR-2, anti-TLR-4, anti-TLR-5 or anti-CLEC9A.

98. The kit according to embodiment 97, wherein the targeting unit comprises or consists of human MIP-1α (LD78B, CCL3L1).

99. The kit according to embodiment 98, wherein the targeting unit comprises an amino acid sequence having at least 80% sequence identity to the amino acid sequence 24-93 of SEQ ID NO: 1.

100. The kit according to embodiment 99, wherein the targeting unit consists of an amino acid sequence having at least 80% sequence identity to the amino acid sequence 24-93 of SEQ ID NO: 1.

101. The kit according to embodiment 100, wherein the targeting unit consist of the amino acid sequence 24-93 of SEQ ID NO: 1.

102. The kit according to any of embodiments 70 to 101, wherein the multimerization unit, such as a dimerization unit, comprises a hinge region, such as hinge exon hl and hinge exon h4.

103. The kit according to embodiment 102, wherein the hinge region has the ability to form one or more covalent bonds.

104. The kit according to any of embodiments 102 to 103, wherein the hinge region is

Ig derived.

105. The kit according to any of embodiments 102 to 104, wherein the anticancer vaccine comprises a dimerization unit and said dimerization unit further comprises another domain that facilitates dimerization.

106. The kit according to embodiment 105, wherein the other domain is an immunoglobulin domain, preferably an immunoglobulin constant domain.

107. The kit according to any of embodiments 105 to 106, wherein the other domain is a carboxyterminal C domain derived from IgG, preferably from IgG3.

108. The kit according to any of embodiments 105 to 107, wherein the dimerization unit further comprises a dimerization unit linker, such as glycine-serine rich linker, such as GGGSSGGGSG (SEQ ID NO: 118).

109. The kit according to embodiment 108, wherein the dimerization unit linker connects the hinge region and the other domain that facilitates dimerization.

110. The kit according to any of embodiments 105 to 109, wherein the dimerization unit comprises hinge exon h1 and hinge exon h4, a dimerization unit linker and a CH3 domain of human IgG3.

111. The kit according to embodiment 110, wherein the dimerization unit comprises an amino acid sequence having at least 80% sequence identity to the amino acid sequence 94-237 of SEQ ID NO: 1.

112. The kit according to embodiment 111, wherein the dimerization unit consists of an amino acid sequence having at least 80% sequence identity to the amino acid sequence 94-237 of SEQ ID NO: 1.

113. The kit according to embodiment 112, wherein the dimerization unit consists of the amino acid sequence 94-237 of SEQ ID NO: 1.

114. The kit according to any of embodiments 70 to 113, wherein the anticancer vaccine further comprises a unit liker that connects the antigenic unit to the multimerization unit, such as dimerization unit and wherein the unit linker is a non-immunogenic linker and/or flexible or rigid linker.

115. The kit according to any of embodiments 70 to 114, wherein the anticancer vaccine is a polynucleotide, preferably an RNA or DNA.

116. The kit according to embodiment 115, wherein the polynucleotide further comprises a nucleotide sequence encoding a signal peptide.

117. The kit according to embodiment 116, wherein the signal peptide is selected from the list consisting of Ig VH signal peptide, human TPA signal peptide and human MIP-1α signal peptide.

118. The kit according to any of embodiments 116 to 117, wherein the signal peptide comprises an amino acid sequence having at least 85% sequence identity to the amino acid sequence 1-23 of SEQ ID NO: 1.

119. The kit according to embodiment 118, wherein the signal peptide consists of an amino acid sequence having at least 85% sequence identity to the amino acid sequence 1-23 of SEQ ID NO: 1.

120. The kit according to embodiment 119, wherein the signal peptide consists of the amino acid sequence 1-23 of SEQ ID NO: 1.

121. The kit according to any of embodiments 115 to 120, wherein the polynucleotide is a DNA.

122. The kit according to embodiment 121, wherein the polynucleotide is a DNA, which is comprised in a vector.

123. The kit according to any of embodiments 70 to 114, wherein the anticancer vaccine is a polypeptide or multimeric protein, such as a dimeric protein.

124. The kit according to any of embodiments 70 to 123, wherein the kit further comprises one or more pharmaceutically acceptable carriers or diluents.

125. The kit according to embodiment 124, comprising a first container comprising the anticancer vaccine and a second container comprising the pharmaceutically acceptable carrier or diluent.

126. The kit according to embodiment 125, wherein the anticancer vaccine is a polypeptide or multimeric protein, such as a dimeric protein, and the pharmaceutically acceptable carrier or diluent further comprises an adjuvant.

127. The kit according to any of embodiments 125 to 126, wherein the kit comprises several such first containers and one or several such second containers.

128. The kit according to embodiment 124, comprising a container comprising the anticancer vaccine and the pharmaceutically acceptable carrier or diluent.

129. The kit according to embodiment 128, comprising several such containers.

130. The kit according to any of embodiments 70 to 129, wherein the one or more checkpoint inhibitor is selected from the group consisting of an anti-PD-L1 antibody, an anti-TIGIT antibody, an anti-CTLA-4-antibody and an anti-PD1-antibody, such as wherein the checkpoint inhibitor is an anti-PD-L1 antibody or an anti-TIGIT antibody or an anti-CTLA-4-antibody or an anti-PD1-antibody or wherein the checkpoint inhibitors are an anti-PD-L1 antibody and an anti-TIGIT antibody or an anti-PD-L1 antibody and an anti-CTLA-4-antibody or an anti-PD-1 antibody and an anti-TIGIT antibody or an anti-PD-1 antibody and an anti-CTLA-4-antibody or an anti-TIGIT antibody and an anti-CTLA-4-antibody or an anti-PD-L1 antibody and an anti-PD-1 antibody or wherein the checkpoint inhibitors are an anti-PD-L1 antibody, an anti-PD-1 antibody and an anti-CTLA-4-antibody or an anti-PD-L1 antibody, an anti-PD-1 antibody and an anti-TIGIT antibody or an anti-PD-L1 antibody, an anti-CTLA-4-antibody and an anti-TIGIT antibody or an anti-PD-1 antibody, an anti-CTLA-4-antibody and an anti-TIGIT antibody or wherein the checkpoint inhibitors are anti-PD-L1 antibody, anti-TIGIT antibody, anti-CTLA-4-antibody and anti-PD1-antibody.

131. The kit according to embodiment 130, wherein the checkpoint inhibitor is an anti-PD-L1 antibody.

132. The kit according to embodiment 130, wherein the checkpoint inhibitor is an anti-TIGIT antibody.

133. The kit according to embodiment 130, wherein the checkpoint inhibitors are an anti-PD-L1 antibody and an anti-TIGIT antibody.

134. The kit according to embodiment 130, wherein the checkpoint inhibitor is an anti-CTLA-4 antibody.

135. The kit according to any of embodiments 70 to 134, comprising a container comprising a checkpoint inhibitor and a container comprising a liquid suitable for injection to a subject, such as infusion injection to a subject.

136. The kit according to embodiment 135, comprising several containers, each comprising a checkpoint inhibitor, and a container comprising a liquid suitable for injection to a subject, such as infusion injection to a subject.

137. The kit according to embodiment 135, comprising several containers, each comprising a checkpoint inhibitor, and several containers comprising a liquid suitable for injection to a subject, such as infusion injection to a subject.

138. The kit according to any of embodiments 70 to 134, comprising a container comprising a checkpoint inhibitor and a liquid suitable for injection to a subject, such as infusion injection to a subject.

139. The kit according to embodiment 138, comprising several containers.

140. The kit according to any of embodiments 70 to 139, wherein the kit further comprises instructions for use.

141. The kit according to embodiment 140, wherein the instructions for use include instructions for the reconstitution of (a) and (b) and/or, instructions for determining a suitable dose of (a) and (b) and/or instructions for the administration of (a) and (b) and/or instructions for the frequency and schedule of administering (a) and (b).

142. The kit according to any of embodiments 70 to 141 for use in a method of treating a subject having cancer, wherein the method comprises the administration of the anticancer vaccine and the one or more checkpoint inhibitors comprised in the kit to the subject.

143. A kit comprising
(a) an anticancer vaccine comprised in one or more first containers, wherein the anticancer vaccine comprises
(i) a polynucleotide comprising a nucleotide sequence encoding a targeting unit that targets antigen-presenting cells, a multimerization unit, such as a dimerization unit, and an antigenic unit comprising one or more cancer antigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors comprised in one or more second containers.

144. The kit according to embodiment 143, wherein the anticancer vaccine is an individualized anticancer vaccine.

145. The kit according to embodiment 144, wherein the antigenic unit comprises one or more neoantigens or parts thereof.

146. The kit according to embodiment 145, wherein the antigenic unit comprises one or more parts of one or more neoantigens.

147. The kit according to embodiment 146, wherein said parts are neoepitopes.

148. The kit according to embodiment 147, wherein the antigenic unit comprises several neoepitopes, such as several neoepitopes which are separated from each other by linkers.

149. The kit according to any of embodiments 147 and 148, wherein the antigenic unit comprises n−1 antigenic subunits, each subunit comprising a neoepitope and a subunit linker, and a terminal neoepitope, and wherein n is the number of neoepitopes in said antigenic unit and n is an integer of from 1 to 50.

150. The kit according to any of embodiments 147 to 149, wherein the neoepitopes have a length of from 7 to 30 amino acids, such as from 7 to 10 amino acids (such as 7, 8, 9 or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

151. The kit according to any of embodiments 145 to 150, wherein the antigenic unit further comprises one or more patient-present shared cancer antigens or parts thereof.

152. The kit according to embodiment 151, wherein the antigenic unit further comprises one or more parts of one or more patient-present shared cancer antigens.

153. The kit according to embodiment 152, wherein said parts are epitopes.

154. The kit according to embodiment 153, wherein the antigenic unit further comprises several epitopes.

155. The kit according to any of embodiments 151 to 154, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

156. The kit according to embodiment 144, wherein the antigenic unit comprises one or more patient-present shared cancer antigens or parts thereof.

157. The kit according to embodiment 156, wherein the antigenic unit comprises one or more parts of one or more patient-present shared cancer antigens.

158. The kit according to embodiment 157, wherein said parts are epitopes.

159. The kit according to embodiment 158, wherein the antigenic unit comprises several epitopes.

160. The kit according to any of embodiments 156 to 159, wherein the patient-present shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations.

161. The kit according to embodiment 143, wherein the anticancer vaccine is a non-individualized anticancer vaccine.

162. The kit according to embodiment 161, wherein the antigenic unit comprises one or more shared cancer antigens or parts thereof.

163. The kit according to embodiment 162, wherein the antigenic unit comprises one or more parts of one or more shared cancer antigens.

164. The kit according to embodiment 163, wherein said parts are epitopes.

165. The kit according to embodiment 164, wherein the antigenic unit comprises several epitopes.

166. The kit according to any of embodiments 162 to 165, wherein the shared cancer antigen is selected from the group consisting of overexpressed or aberrantly expressed human cellular proteins, cancer testis antigens, differentiation antigens, viral antigens, mutated oncogenes, mutated tumor suppressor genes, oncofetal antigens, shared intron retention antigens, shared antigens caused by frameshift mutation, dark matter antigens and shared antigens caused by spliceosome mutations, scFvs derived from a monoclonal Ig produced by myeloma or lymphoma, telomerase, HIV antigens, tyrosinase, tyrosinase related protein (TRP)-1, TRP-2, melanoma antigen, prostate specific antigen and HPV antigens.

167. The kit according to any of embodiments 143 to 166, wherein the antigenic unit comprises up to 3500 amino acids, such as from about 21 to about 2000 amino acids or from about 60 to 3500 amino acids, such as from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

168. The kit according to any of embodiments 143 to 167, wherein the targeting unit is or comprises a moiety that interacts with surface molecules on the antigen-presenting cells.

169. The kit according to embodiment 168, wherein the surface molecule is selected from the group consisting of HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5.

170. The kit according to any of embodiments 168 and 169, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II anti-CD40, anti-TLR-2, anti-TLR-4, anti-TLR-5 or anti-CLEC9A.

171. The kit according to embodiment 170, wherein the targeting unit comprises or consists of human MIP-1α (LD78B, CCL3L1).

172. The kit according to embodiment 171, wherein the targeting unit comprises an amino acid sequence having at least 80% sequence identity to the amino acid sequence 24-93 of SEQ ID NO: 1.

173. The kit according to embodiment 172, wherein the targeting unit consists of an amino acid sequence having at least 80% sequence identity to the amino acid sequence 24-93 of SEQ ID NO: 1.

174. The kit according to embodiment 173, wherein the targeting unit consist of the amino acid sequence 24-93 of SEQ ID NO: 1.

175. The kit according to any of embodiments 143 to 174, wherein the multimerization unit, such as a dimerization unit, comprises a hinge region, such as hinge exon hl and hinge exon h4.

176. The kit according to embodiment 175, wherein the hinge region has the ability to form one or more covalent bonds.

177. The kit according to any of embodiments 175 to 176, wherein the hinge region is Ig derived.

178. The kit according to any of embodiments 175 to 177, wherein the anticancer vaccine comprises a dimerization unit and said dimerization unit further comprises another domain that facilitates dimerization.

179. The kit according to embodiment 178, wherein the other domain is an immunoglobulin domain, preferably an immunoglobulin constant domain.

180. The kit according to any of embodiments 178 to 179, wherein the other domain is a carboxyterminal C domain derived from IgG, preferably from IgG3.

181. The kit according to any of embodiments 178 to 180, wherein the dimerization unit further comprises a dimerization unit linker, such as glycine-serine rich linker, such as GGGSSGGGSG (SEQ ID NO: 118).

182. The kit according to embodiment 181, wherein the dimerization unit linker connects the hinge region and the other domain that facilitates dimerization.

183. The kit according to any of embodiments 178 to 182, wherein the dimerization unit comprises hinge exon h1 and hinge exon h4, a dimerization unit linker and a CH3 domain of human IgG3.

184. The kit according to embodiment 183, wherein the dimerization unit comprises an amino acid sequence having at least 80% sequence identity to the amino acid sequence 94-237 of SEQ ID NO: 1.

185. The kit according to embodiment 184, wherein the dimerization unit consists of an amino acid sequence having at least 80% sequence identity to the amino acid sequence 94-237 of SEQ ID NO: 1.

186. The kit according to embodiment 185, wherein the dimerization unit consists of the amino acid sequence 94-237 of SEQ ID NO: 1.

187. The kit according to any of embodiments 143 to 186, wherein the anticancer vaccine further comprises a unit liker that connects the antigenic unit to the multimerization unit, such as dimerization unit and wherein the unit linker is a non-immunogenic linker and/or flexible or rigid linker.

188. The kit according to any of embodiments 143 to 187, wherein the anticancer vaccine is a polynucleotide, preferably an RNA or DNA.

189. The kit according to embodiment 188, wherein the polynucleotide further comprises a nucleotide sequence encoding a signal peptide.

190. The kit according to embodiment 189, wherein the signal peptide is selected from the list consisting of Ig VH signal peptide, human TPA signal peptide and human MIP-1α signal peptide.

191. The kit according to any of embodiments 189 to 190, wherein the signal peptide comprises an amino acid sequence having at least 85% sequence identity to the amino acid sequence 1-23 of SEQ ID NO: 1.

192. The kit according to embodiment 191, wherein the signal peptide consists of an amino acid sequence having at least 85% sequence identity to the amino acid sequence 1-23 of SEQ ID NO: 1.

193. The kit according to embodiment 192, wherein the signal peptide consists of the amino acid sequence 1-23 of SEQ ID NO: 1.

194. The kit according to any of embodiments 188 to 193, wherein the polynucleotide is a DNA.

195. The kit according to embodiment 194, wherein the polynucleotide is a DNA, which is comprised in a vector.

196. The kit according to any of embodiments 143 to 195, wherein the anticancer vaccine is a polypeptide or multimeric protein, such as a dimeric protein.

197. The kit according to any of embodiments 143 to 196, wherein the one or more checkpoint inhibitor is selected from the group consisting of an anti-PD-L1 antibody, an anti-TIGIT antibody, an anti-CTLA-4-antibody and an anti-PD1-antibody, such as wherein the checkpoint inhibitor is an anti-PD-L1 antibody or an anti-TIGIT antibody or an anti-CTLA-4-antibody or an anti-PD1-antibody or wherein the checkpoint inhibitors are an anti-PD-L1 antibody and an anti-TIGIT antibody or an anti-PD-L1 antibody and an anti-CTLA-4-antibody or an anti-PD-1 antibody and an anti-TIGIT antibody or an anti-PD-1 antibody and an anti-CTLA-4-antibody or an anti-TIGIT antibody and an anti-CTLA-4-antibody or an anti-PD-L1 antibody and an anti-PD-1 antibody or wherein the checkpoint inhibitors are an anti-PD-L1 antibody, an anti-PD-1 antibody and an anti-CTLA-4-antibody or an anti-PD-L1 antibody, an anti-PD-1 antibody and an anti-TIGIT antibody or an anti-PD-L1 antibody, an anti-CTLA-4-antibody and an anti-TIGIT antibody or an anti-PD-1 antibody, an anti-CTLA-4-antibody and an anti-TIGIT antibody or wherein the checkpoint inhibitors are anti-PD-L1 antibody, anti-TIGIT antibody, anti-CTLA-4-antibody and anti-PD1-antibody.

198. The kit according to embodiment 197, wherein the checkpoint inhibitor is an anti-PD-L1 antibody.

199. The kit according to embodiment 197, wherein the checkpoint inhibitor is an anti-TIGIT antibody.

200. The kit according to embodiment 197, wherein the checkpoint inhibitors are an anti-PD-L1 antibody and an anti-TIGIT antibody.

201. The kit according to embodiment 197, wherein the checkpoint inhibitors is an anti-CTLA-4 antibody.

202. The kit according to any of embodiments 143 to 201, wherein the anticancer vaccine further comprises a pharmaceutically acceptable carrier or diluent and wherein the one or more checkpoint inhibitor is comprised in a composition suitable for injection, such as infusion injection.

203. The kit according to any of embodiments 143 to 202, wherein said kit further comprises one or more third containers comprising pharmaceutically acceptable carriers or diluents.

204. The kit according to any of embodiments 143 to 203, wherein said kit further comprises instructions for use, such as instructions for the reconstitution of a dose of (a) and (b), and/or instructions for determining a suitable dose of (a) and (b) and/or instructions for the administration of (a) and (b), and/or instructions for the frequency and schedule of administering (a) and (b).

205. The kit according to any of embodiments 143 to 204 for use in a method of treating a subject having cancer, wherein the method comprises the administration of the anticancer vaccine and the one or more checkpoint inhibitors comprised in the kit to the subject.

205. A method for treating a subject having cancer, the method comprising administering to the subject
(a) an individualized DNA anticancer vaccine comprising
(i) a DNA polynucleotide, such as VB10.NEO, comprising a nucleotide sequence encoding a targeting unit that targets antigen-presenting cells, a dimerization unit, and an antigenic unit comprising one or more neoepitopes; and
(b) an anti-PD-L1 antibody, such as atezolizumab.

206. The method according to embodiment 205, wherein the targeting unit is or comprises a moiety that interacts with surface molecules on the antigen-presenting cells.

207. The method according to embodiment 206, wherein the surface molecule is selected from the group consisting of HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5.

208. The method according to any of embodiments 206 and 207, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II anti-CD40, anti-TLR-2, anti-TLR-4, anti-TLR-5 or anti-CLEC9A..

209. The method according to embodiment 208, wherein the targeting unit comprises or consists of human MIP-1α (LD78B, CCL3L1).

210. The method according to embodiment 209, wherein the targeting unit comprises an amino acid sequence having at least 80% sequence identity to the amino acid sequence 24-93 of SEQ ID NO: 1, such as comprising the amino acid sequence 26-93 of SEQ ID NO: 1 or comprising the amino acid sequence 28-93 of SEQ ID NO: 1.

211. The method according to embodiment 210 wherein the targeting unit consists of an amino acid sequence having at least 80% sequence identity to the amino acid sequence 24-93 of SEQ ID NO: 1, such as consisting of the amino acid sequence 26-93 of SEQ ID NO: 1 or consisting of the amino acid sequence 28-93 of SEQ ID NO: 1.

212. The method according to embodiment 211, wherein the targeting unit consists of the amino acid sequence 24-93 of SEQ ID NO: 1.

213. The method according to any of embodiments 205 to 212, wherein the dimerization unit comprises a hinge region, such as hinge exon h1 and hinge exon h4.

214. The method according to embodiment 213, wherein the hinge region has the ability to form one or more covalent bonds.

215. The method according to any of embodiments 213 to 214, wherein the hinge region is Ig derived.

216. The method according to any of embodiments 213 to 215, wherein the dimerization unit further comprises another domain that facilitates dimerization.

217. The method according to embodiment 216, wherein the other domain is an immunoglobulin domain, preferably an immunoglobulin constant domain.

218. The method according to any of embodiments 216 to 217, wherein the other domain is a carboxyterminal C domain derived from IgG, preferably from IgG3.

219. The method according to any of embodiments 216 to 218, wherein the dimerization unit further comprises a dimerization unit linker, such as glycine-serine rich linker, such as GGGSSGGGSG (SEQ ID NO: 118).

220. The method according to embodiment 219, wherein the dimerization unit linker connects the hinge region and the other domain that facilitates dimerization.

221. The method according to any of embodiments 216 to 220, wherein the dimerization unit comprises hinge exon h1 and hinge exon h4, a dimerization unit linker and a CH3 domain of human IgG3.

222. The method according to embodiment 221, wherein the dimerization unit comprises an amino acid sequence having at least 80% sequence identity to the amino acid sequence 94-237 of SEQ ID NO: 1.

223. The method according to embodiment 222, wherein the dimerization unit consists of an amino acid sequence having at least 80% sequence identity to the amino acid sequence 94-237 of SEQ ID NO: 1.

224. The method according to embodiment 223, wherein the dimerization unit consists of the amino acid sequence 94-237 of SEQ ID NO: 1.

225. The method according to any of embodiments 205 to 224, wherein the anticancer vaccine further comprises a unit liker that connects the antigenic unit to the dimerization unit, and wherein the unit linker is a non-immunogenic linker and/or flexible or rigid linker.

226. The method according to embodiment 205 to 225, wherein the DNA further comprises a nucleotide sequence encoding a signal peptide.

227. The method according to embodiment 226, wherein the signal peptide is selected from the list consisting of Ig VH signal peptide, human TPA signal peptide and human MIP-1α signal peptide.

228. The method according to any of embodiments 226 to 227, wherein the signal peptide comprises an amino acid sequence having at least 85% sequence identity to the amino acid sequence 1-23 of SEQ ID NO: 1.

229. The method according to embodiment 228, wherein the signal peptide consists of an amino acid sequence having at least 85% sequence identity to the amino acid sequence 1-23 of SEQ ID NO: 1.

230. The method according to embodiment 229, wherein the signal peptide consists of the amino acid sequence 1-23 of SEQ ID NO: 1.

231. The method according to embodiment 205 to 23, wherein the DNA polynucleotide comprises a nucleotide sequence encoding a polypeptide which comprises an amino acid sequence having at least 80% sequence identity to the amino acid sequence 1-242 of SEQ ID NO: 1, such as a polypeptide which comprises the amino acid sequence 1-242 of SEQ ID NO: 1.

232. The method according to embodiment 231, wherein the DNA polynucleotide comprises a nucleotide sequence encoding a polypeptide which consists of an amino acid sequence having at least 80% sequence identity to the amino acid sequence 1-242 of SEQ ID NO: 1, such as a polypeptide which consists of the amino acid sequence 1-242 of SEQ ID NO: 1.

233. The method according to any of embodiments 205 to 232, wherein the DNA polynucleotide comprises a constant part and a variable part, wherein the constant part consists of a nucleotide sequence encoding a polypeptide which consists of the amino acid sequence 1-242 of SEQ ID NO: 1

234. The method according to any of embodiments 205 to 233, wherein the DNA polynucleotide is comprised in a vector.

235. The method according to any of embodiments 205 to 234, wherein the anticancer vaccine further comprises a pharmaceutically acceptable carrier or diluent.

236. The method according to any of embodiments 205 to 235 wherein the anti-PD-L1 antibody is comprised in an aqueous, liquid composition for injection, such as infusion injection.

237. The method according to any of embodiments 205 to 236, wherein the anti-PD-LI antibody is administered by infusion injection.

238. The method according to any of embodiments 205 to 237, wherein the anticancer vaccine is administered intramuscularly.

239. The method according to any of embodiments 205 to 237, wherein the cancer is a solid cancer.

240. A method for treating a subject having a solid cancer, the method comprising administering to the subject
(a) by intramuscular administration an individualized DNA anticancer vaccine comprising a pharmaceutically acceptable carrier and a DNA polynucleotide, such as VB10.NEO, wherein the DNA polynucleotide comprises a constant part consisting of a nucleotide sequence encoding a polypeptide which consists of the amino acid sequence 1-242 of SEQ ID NO: 1 and a variable part, consisting of a nucleotide sequence encoding a polypeptide consisting of one or more neoepitopes separated by linkers; and
(b) by infusion injection an anti-PD-L1 antibody, such as atezolizumab,

241. A method for treating a patient having cancer, the method comprising administering to the patient an effective amount of an individualized DNA anticancer vaccine comprising VB10.NEO in combination with atezolizumab, wherein
the patient has at least one of the following tumor types: melanoma, NSCLC, RCC, UC, HNSCC, TNBC, gastric/GEJ cancer, cervical, anal, or MSI-high tumors; and
wherein the patient has a locally advanced or metastatic tumor that has progressed after at least 1 available standard therapy; and/or for whom standard therapy has proven to be ineffective or intolerable, or is considered inappropriate;
   and/or or for whom a clinical trial of an investigational agent is a recognized standard of care; and
the effective amount is a) VB10.NEO (3 mg in a pharmaceutically acceptable carrier) administered by intramuscular injection for an induction course Q3W (4 doses) followed by maintenance doses Q6W (6 doses) and Q12W (5 doses) and atezolizumab 1200 mg is administered by intravenous infusion on day 1 of 21 day cycles; or b) VB10.NEO (6 mg in a pharmaceutically acceptable carrier) administered by intramuscular injection for an induction course Q3W (4 doses) followed by maintenance doses Q6W (6 doses) and Q12W (5 doses) and atezolizumab 1200 mg is administered by intravenous infusion on day 1 of 21 day cycles; and
the method for treating results in an improved overall response rate (ORR) and/or an improved duration of response (DOR) and/or an improved progression free survival (PFS) and/or an improved overall survival (OS) compared to a reference population, and, optionally, the reference population is one treated with a Standard of Care (SOC) for the tumor.

1. A method for treating a subject having cancer, the method comprising administering to the subject
(a) an anticancer vaccine comprising
(i) a polynucleotide comprising a nucleotide sequence encoding a targeting unit that targets antigen-presenting cells, a multimerization unit, such as a dimerization unit, and an antigenic unit comprising one or more cancer antigens or parts thereof, or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors.

2. The method according to claim 1, wherein the anticancer vaccine is an individualized anticancer vaccine.

3. The method according to claim 2, wherein the antigenic unit comprises one or more neoantigens or parts thereof, such as one or more neoepitopes.

4. The method according to claim 3, wherein the antigenic unit comprises several neoepitopes which are separated from each other by linkers.

5. The method according to any of claims 3 to 4, wherein the antigenic unit further comprises one or more patient-present shared cancer antigens or parts thereof, such as epitopes, e.g. several epitopes.

6. The method according to claim 2, wherein the antigenic unit comprises one or more patient-present shared cancer antigens or parts thereof, such as epitopes, e.g. several epitopes.

7. The method according to claim 1, wherein the anticancer vaccine is a non-individualized anticancer vaccine.

8. The method according to claim 7, wherein the antigenic unit comprises one or more shared cancer antigens or parts thereof, such as epitopes, such as several epitopes.

9. The method according to any of claims 1 to 8, wherein the one or more checkpoint inhibitor is selected from the group consisting of anti-PD-L1 antibody, an anti-TIGIT antibody, and an anti-CTLA-4-antibody.

10. The method according to claim 9, wherein the checkpoint inhibitor is an anti-CTLA-4-antibody.

11. The method according to claim 9, wherein the checkpoint inhibitors are an anti-PD-L1 antibody and an anti-TIGIT antibody.

12. The method according to any of claims 1 to 11, wherein the targeting unit is or comprises a moiety that interacts with surface molecules on the antigen-presenting cells, such as a surface molecule selected from the group consisting of HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5.

13. The method according to claim 12, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II anti-CD40, anti-TLR-2, anti-TLR-4, anti-TLR-5 or anti-CLEC9A.

14. The method according to any of claims 1 to 13, wherein the multimerization unit is a trimerization unit, such as the C-terminal domain of T4 fibritin or such as a collagen-derived trimerization unit, such as human collagen derived XVIII trimerization domain or human collagen XV trimerization domain, or a tetramerization unit, such as a domain derived from p53.

15. The method according to any of claims 1 to 13, wherein the multimerization unit is a dimerization unit, such as a dimerization unit comprising a hinge region and an immunoglobulin domain, e.g. an immunoglobulin constant domain or a dimerization unit comprising the dHLX protein.

16. The method according to any of claims 1 to 15, wherein the anticancer vaccine further comprises a unit liker that connects the antigenic unit to the multimerization unit, such as dimerization unit, and wherein the unit linker is a non-immunogenic linker and/or flexible or rigid linker.

17. The method according to any of claims 1 to 16, wherein the anticancer vaccine is a polynucleotide, such as an RNA or DNA, e.g. an RNA or DNA comprised in a vector.

18. The method according to claim 17, wherein the polynucleotide further comprises a nucleotide sequence encoding a signal peptide.

19. The method according to claim 18, wherein the anticancer vaccine comprises a DNA polynucleotide encoding a human MIP-1α signal peptide, a human MIP-1α targeting unit, a dimerization unit which comprises a hinge exon h1, a hinge exon h4, a dimerization unit linker and a CH3 domain of human IgG3, a unit linker and an antigenic unit comprising one or more neoepitopes.

20. The method according to claim 19, wherein the checkpoint inhibitor is an anti-PD-LI antibody or an anti-TIGIT antibody or an anti-CTLA4 antibody or wherein the checkpoint inhibitors are an anti-PD-L1 antibody and an anti-TIGIT antibody.

21. The method according to any of claims 1 to 20, wherein the anticancer vaccine further comprises a pharmaceutically acceptable carrier or diluent and wherein the one or more checkpoint inhibitor is comprised in a composition suitable for injection, such as infusion injection.

22. The method according to any of claims 1 to 21, wherein the one or more checkpoint inhibitors are administered concurrently with the anticancer vaccine or prior to the first administration of the anticancer vaccine or wherein the anticancer vaccine is administered prior to the administration of the one or more checkpoint inhibitors.

23. The method according to any of claims 1 to 22, wherein the cancer is a solid cancer or a liquid cancer, such as a cancer selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

24. A kit comprising
(a) an anticancer vaccine comprised in one or more first containers, wherein the anticancer vaccine comprises
(i) a polynucleotide comprising a nucleotide sequence encoding a targeting unit that targets antigen-presenting cells, a multimerization unit, such as a dimerization unit, and an antigenic unit comprising one or more cancer antigens or parts thereof; or
(ii) a polypeptide encoded by the polynucleotide as defined in (i); or
(iii) a multimeric protein, such as a dimeric protein, consisting of multiple polypeptides as defined in (ii), such as of two polypeptides; and
(b) one or more checkpoint inhibitors comprised in one or more second containers.

25. The kit according to claim 24, wherein the anticancer vaccine is an individualized anticancer vaccine.

26. The kit according to claim 25, wherein the antigenic unit comprises one or more neoantigens or parts thereof, such as one or more neoepitopes.

27. The kit according to claim 26, wherein the antigenic unit comprises several neoepitopes which are separated from each other by linkers.

28. The kit according to any of claims 26 to 27, wherein the antigenic unit further comprises one or more patient-present shared cancer antigens or parts thereof, such as epitopes, e.g. several epitopes.

29. The kit according to claim 28, wherein the antigenic unit comprises one or more patient-present shared cancer antigens or parts thereof, such as epitopes, e.g. several epitopes.

30. The kit according to claim 24, wherein the anticancer vaccine is a non-individualized anticancer vaccine.

31. The kit according to claim 30, wherein the antigenic unit comprises one or more shared cancer antigens or parts thereof, such as epitopes, such as several epitopes.

32. The kit according to any of claims 24 to 31, wherein the one or more checkpoint inhibitor is selected from the group consisting of an anti-PD-L1 antibody, an anti-TIGIT antibody, and an anti-CTLA-4-antibody.

33. The kit according to claim 32, wherein the checkpoint inhibitor is an anti-CTLA-4-antibody.

34. The kit according to claim 32, wherein the checkpoint inhibitors are an anti-PD-L1 and an anti-TIGIT antibody.

35. The kit according to any of claims 24 to 34, wherein the targeting unit is or comprises a moiety that interacts with surface molecules on the antigen-presenting cells, such as a surface molecule selected from the group consisting of HLA, CD14, CD40, CLEC9A, chemokine receptors, such as CCR1, CCR3, CCR4, CCR5, CCR6, CCR7, CCR8 or XCR1 and Toll-like receptors such as TLR-2, TLR-4 or TLR-5.

36. The kit according to claim 35, wherein the targeting unit comprises or consists of soluble CD40 ligand, CCL4 and its isoforms, CCL5, CCL19, CCL20, CCL21, macrophage inflammatory protein alpha including its isoforms, such as mouse CCL3, human CCL3, human CCL3L1, human CCL3L2 and human CCL3L3, XCL1, XCL2, flagellin, anti-HLA-DP, anti-HLA-DR, anti-pan HLA class II anti-CD40, anti-TLR-2, anti-TLR-4, anti-TLR-5 or anti-CLEC9A.

37. The kit according to any of claims 24 to 36, wherein the multimerization unit is a trimerization unit, such as the C-terminal domain of T4 fibritin or such as a collagen-derived trimerization unit, such as human collagen derived XVIII trimerization domain or human collagen XV trimerization domain, or a tetramerization unit, such as a domain derived from p53.

38. The kit according to any of claims 24 to 36, wherein the multimerization unit is a dimerization unit, such as a dimerization unit comprising a hinge region and an immunoglobulin domain, e.g. an immunoglobulin constant domain or a dimerization unit comprising the dHLX protein.

39. The kit according to any of claims 24 to 38, wherein the anticancer vaccine further comprises a unit liker that connects the antigenic unit to the multimerization unit, such as dimerization unit, and wherein the unit linker is a non-immunogenic linker and/or flexible or rigid linker.

40. The kit according to any of claims 24 to 39, wherein the anticancer vaccine is a polynucleotide, such as an RNA or DNA, e.g. an RNA or DNA comprised in a vector.

41. The kit according to claim 40, wherein the polynucleotide further comprises a nucleotide sequence encoding a signal peptide.

42. The kit according to claim 41, wherein the anticancer vaccine comprises a DNA polynucleotide encoding a human MIP-1α signal peptide, a human MIP-1α targeting unit, a dimerization unit which comprises a hinge exon h1, a hinge exon h4, a dimerization unit linker and a CH3 domain of human IgG3, a unit linker and an antigenic unit comprising one or more neoepitopes.

43. The kit according to claim 42, wherein the checkpoint inhibitor is an anti-PD-L1 antibody or an anti-TIGIT antibody or an anti-CTLA4 antibody or wherein the checkpoint inhibitors are an anti-PD-L1 antibody and an anti-TIGIT antibody.

44. The kit according to any of claims 24 to 43, wherein the anticancer vaccine further comprises a pharmaceutically acceptable carrier or diluent and wherein the one or more checkpoint inhibitor is comprised in a composition suitable for injection, such as infusion injection.

45. The kit according to any of claims 24 to 44, wherein said kit further comprises one or more third containers comprising pharmaceutically acceptable carriers or diluents.

46. The kit according to any of claims 24 to 45, wherein said kit further comprises instructions for use, such as instructions for the reconstitution of a dose of (a) and (b), and/or instructions for determining a suitable dose of (a) and (b) and/or instructions for the administration of (a) and (b), and/or instructions for the frequency and schedule of administering (a) and (b).

47. The kit according to any of claims 24 to 46 for use in a method of treating a subject having cancer, wherein the method comprises the administration of the anticancer vaccine and the one or more checkpoint inhibitors comprised in the kit to the subject.


SEQUENCE LISTING (141 sequences)

SEQ ID NO: 1 - protein, 972 aa, Artificial Sequence
  VB4085 / VB1026
  SITE (1)..(972): VB4085 (amino acids 1-972) / VB1026 (amino acids 1-237)
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSTMLYIRALKNPSLYGFSSGLNKDGIEGGGGSGGGGSGALKKLIYAAKLNTSLKALEGERNQVYGGGGSGGGGSDLVCDFQSFKYYAHATSLAGHLVSCPLGGGGSGGGGSEKLRNPCPNKEKAYQPPFAFRHVLKLTGGGGSGGGGSDTLSAMSNPRAMQVLLQIQQGLQTLATGGGGSGGGGSGDVKIHAHKVVLANISPYFKAMFTGNLGGGGSGGGGSEVIQTSKYYMRDVIAIESAWLLELAPHGGGGSGGGGSFFSFFTHRFGHHVSPQVHIILANLYLLGGGGSGGGGSVILPQAPSGPSYATYLQPAQAQMLTPPGGGGSGGGGSLWVYLRPVPRPATIYLQILRLKPLTGEGGGGSGGGGSFVSPMAHYVPGIMAIESVVARFQFIVPGGGGSGGGGSTLAFLVLSTPAMFNRALKPFLKSCHFQGGGGSGGGGSFLERPMDMPYMIFYPNNPLMTGQLLGSGGGGSGGGGSIPREVGDGTRVDPFPPVQTWMRLPKLVGGGGSGGGGSGSLFGSSRVQYVVNPAVKIVFLNIDPSGGGGSGGGGSNNLQKYIEIYVQKINPSRLPVVIGGLLGGGGSGGGGSAEYGDYQPEVHGVPYFRLEHYLPARVMGGGGSGGGGSTPLRKHTVHAIRKFYLEFKGSSPPPRLGGGGSGGGGSKIYEFDYHLYGQNITMIMTSVSGHLLAGGGGSGGGGSKSWIHCWKYLSVQSQLFRGSSLLFRRV

SEQ ID NO: 2 - protein, 19 aa, Artificial Sequence
  Signal peptide
  SITE (1)..(19): Signal peptide
MNFGLRLIFLVLTLKGVQC

SEQ ID NO: 3 - protein, 22 aa, Artificial Sequence
  Signal peptide
  SITE (1)..(22): Signal peptide
MDAMKRGLCCVLLLCGAVFVSP

SEQ ID NO: 4 - DNA, 69 bp, Artificial Sequence
  Nucleotide sequence
  misc_feature (1)..(69): Nucleotide sequence encoding amino acids 1-23 of SEQ ID NO 1
atgcaggtctccactgctgcccttgccgtcctcctctgcaccatggctctctgcaaccaggtcctctct

SEQ ID NO: 5 - DNA, 210 bp, Artificial Sequence
  Nucleotide sequence
  misc_feature (1)..(210): Nucleotide sequence encoding amino acids 24-93 of SEQ ID NO 1
gcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgcc

SEQ ID NO: 6 - DNA, 36 bp, Artificial Sequence
  Nucleotide sequence
  misc_feature (1)..(36): Nucleotide sequence encoding amino acids 94-105 of SEQ ID NO 1
gagctcaaaaccccacttggtgacacaactcacaca

SEQ ID NO: 7 - DNA, 45 bp, Artificial Sequence
  Nucleotide sequence
  misc_feature (1)..(45): Nucleotide sequence encoding amino acids 106-120 of SEQ ID NO 1
gagcccaaatcttgtgacacacctcccccgtgcccaaggtgccca

SEQ ID NO: 8 - DNA, 81 bp, Artificial Sequence
  Nucleotide sequence
  misc_feature (1)..(81): Nucleotide sequence encoding amino acids 94-120 of SEQ ID NO 1
gagctcaaaaccccacttggtgacacaactcacacagagcccaaatcttgtgacacacctcccccgtgcccaaggtgccca

SEQ ID NO: 9 - DNA, 30 bp, Artificial Sequence
  Nucleotide sequence
  misc_feature (1)..(30): Nucleotide sequence encoding amino acids 121-130 of SEQ ID NO 1
ggcggtggaagcagcggaggtggaagtgga

SEQ ID NO: 10 - DNA, 321 bp, Artificial Sequence
  Nucleotide sequence
  misc_feature (1)..(321): Nucleotide sequence encoding amino acids 131-237 of SEQ ID NO 1
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 11 - DNA, 15 bp, Artificial Sequence
  Nucleotide sequence
  misc_feature (1)..(15): Nucleotide sequence encoding amino acids 238-242 of SEQ ID NO 1
ggcctcggtggcctg

SEQ ID NO: 12 - DNA, 432 bp, Artificial Sequence
  Nucleotide sequence
  misc_feature (1)..(432): Nucleotide sequence encoding amino acids 94-237 of SEQ ID NO 1
gagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 13 - protein, 447 aa, Artificial Sequence
  Heavy chain
  SITE (1)..(447): Heavy chain amino acid sequence of atezolizumab
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 214 aa, Artificial Sequence
  Light chain
  SITE (1)..(214): Light chain amino acid sequence of atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 10 aa, Artificial Sequence
  CDR
  SITE (1)..(10): HVR-H1 of atezolizumab
GFTFSDSWIH

SEQ ID NO: 16 - protein, 18 aa, Artificial Sequence
  CDR
  SITE (1)..(18): HVR-H21 of atezolizumab
AWISPYGGSTYYADSVKG

SEQ ID NO: 17 - protein, 9 aa, Artificial Sequence
  CDR
  SITE (1)..(9): HVR-H3 of atezolizumab
RHWPGGFDY

SEQ ID NO: 18 - protein, 11 aa, Artificial Sequence
  CDR
  SITE (1)..(11): HVR-L1 of atezolizumab
RASQDVSTAVA

SEQ ID NO: 19 - protein, 7 aa, Artificial Sequence
  CDR
  SITE (1)..(7): HVR-L2 of atezolizumab
SASFLYS

SEQ ID NO: 20 - protein, 9 aa, Artificial Sequence
  CDR
  SITE (1)..(9): HVR-L3 of atezolizumab
QQYLYHPAT

SEQ ID NO: 21 - protein, 118 aa, Artificial Sequence [sequence omitted]

SEQ ID NO: 22 - protein, 108 aa, Artificial Sequence
  HL
  SITE (1)..(108): HL of atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 23 - DNA, 726 bp, Artificial Sequence
  VB10.NEO
  misc_feature (1)..(726)
  misc_feature (1)..(726)
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctg

SEQ ID NO: 24 - protein, 5 aa, Artificial Sequence
  linker
  SITE (1)..(5): subunit linker
GGGGS

SEQ ID NO: 25 - protein, 5 aa, Artificial Sequence
  linker
  SITE (1)..(5): subunit linker
GGGSS

SEQ ID NO: 26 - protein, 5 aa, Artificial Sequence
  linker
  SITE (1)..(5): subunit linker
GGGSG

SEQ ID NO: 27 - protein, 5 aa, Artificial Sequence
  linker
  SITE (1)..(5): subunit linker
GGSGG

SEQ ID NO: 28 - protein, 6 aa, Artificial Sequence
  linker
  SITE (1)..(6): subunit linker
SGSSGS

SEQ ID NO: 29 - protein, 10 aa, Artificial Sequence
  linker
  SITE (1)..(10): subunit linker
GGGGSGGGGS

SEQ ID NO: 30 - protein, 5 aa, Artificial Sequence
  linker
  SITE (1)..(5): subunit linker
  SITE (1)..(5): the sequence can be repeated m times, where m is an integer from 1 to 5, e.g., 1, 2, 3, 4, or 5
GGGGS

SEQ ID NO: 31 - protein, 5 aa, Artificial Sequence
  linker
  SITE (1)..(5): subunit linker
  SITE (1)..(5): the sequence can be repeated m times, where m is an integer from 1 to 5, e.g., 1, 2, 3, 4, or 5
GGGSS

SEQ ID NO: 32 - protein, 5 aa, Artificial Sequence
  linker
  SITE (1)..(5): subunit linker
  SITE (1)..(5): the sequence can be repeated m times, where m is an integer from 1 to 5, e.g., 1, 2, 3, 4, or 5
GGSGG

SEQ ID NO: 33 - protein, 5 aa, Artificial Sequence
  linker
  SITE (1)..(5): subunit linker
  SITE (1)..(5): the sequence can be repeated m times, where m is an integer from 1 to 5, e.g., 1, 2, 3, 4, or 5
GGGSG

SEQ ID NO: 34 - protein, 6 aa, Artificial Sequence
  linker
  SITE (1)..(6): subunit linker
  SITE (1)..(6): the sequence can be repeated m times, where m is an integer from 1 to 5, e.g., 1, 2, 3, 4, or 5
SGSSGS

SEQ ID NO: 35 - protein, 5 aa, Artificial Sequence
  linker
  SITE (1)..(5): subunit linker
LGGGS

SEQ ID NO: 36 - protein, 5 aa, Artificial Sequence
  linker
  SITE (1)..(5): subunit linker
GLGGS

SEQ ID NO: 37 - protein, 5 aa, Artificial Sequence
  linker
  SITE (1)..(5): subunit linker
GGLGS

SEQ ID NO: 38 - protein, 5 aa, Artificial Sequence
  linker
  SITE (1)..(5): subunit linker
GGGLS

SEQ ID NO: 39 - protein, 5 aa, Artificial Sequence
  linker
  SITE (1)..(5): subunit linker
GGGGL

SEQ ID NO: 40 - protein, 5 aa, Artificial Sequence
  linker
  SITE (1)..(5): subunit linker
LGGSG

SEQ ID NO: 41 - protein, 5 aa, Artificial Sequence
  linker
  SITE (1)..(5): subunit linker
GLGSG

SEQ ID NO: 42 - protein, 5 aa, Artificial Sequence
  linker
  SITE (1)..(5): subunit linker
GGLSG

SEQ ID NO: 43 - protein, 5 aa, Artificial Sequence
  linker
  SITE (1)..(5): subunit linker
GGGLG

SEQ ID NO: 44 - protein, 5 aa, Artificial Sequence
  linker
  SITE (1)..(5): subunit linker
GGGSL

SEQ ID NO: 45 - protein, 5 aa, Artificial Sequence
  linker
  SITE (1)..(5): subunit linker
LGGSS

SEQ ID NO: 46 - protein, 5 aa, Artificial Sequence
  linker
  SITE (1)..(5): subunit linker
GLGSS

SEQ ID NO: 47 - protein, 5 aa, Artificial Sequence
  linker
  SITE (1)..(5): subunit linker
GGLSS

SEQ ID NO: 48 - protein, 5 aa, Artificial Sequence
  linker
  SITE (1)..(5): subunit linker
LGLGS

SEQ ID NO: 49 - protein, 5 aa, Artificial Sequence
  linker
  SITE (1)..(5): subunit linker
GLGLS

SEQ ID NO: 50 - protein, 5 aa, Artificial Sequence
  linker
  SITE (1)..(5): subunit linker
GLLGS

SEQ ID NO: 51 - protein, 5 aa, Artificial Sequence
  linker
  SITE (1)..(5): subunit linker
LGGLS

SEQ ID NO: 52 - protein, 5 aa, Artificial Sequence
  linker
  SITE (1)..(5): subunit linker
GLGGL

SEQ ID NO: 53 - protein, 5 aa, Artificial Sequence
  linker
  SITE (1)..(5): subunit linker
LGLSG

SEQ ID NO: 54 - protein, 5 aa, Artificial Sequence
  linker
  SITE (1)..(5): subunit linker
GLLSG

SEQ ID NO: 55 - protein, 5 aa, Artificial Sequence
  linker
  SITE (1)..(5): subunit linker
GGLSL

SEQ ID NO: 56 - protein, 5 aa, Artificial Sequence
  linker
  SITE (1)..(5): subunit linker
GGLLG

SEQ ID NO: 57 - protein, 5 aa, Artificial Sequence
  linker
  SITE (1)..(5): subunit linker
GLGSL

SEQ ID NO: 58 - protein, 5 aa, Artificial Sequence
  linker
  SITE (1)..(5): subunit linker
LGLSS

SEQ ID NO: 59 - protein, 5 aa, Artificial Sequence
  linker
  SITE (1)..(5): subunit linker
GGLLS

SEQ ID NO: 60 - protein, 10 aa, Artificial Sequence
  linker
  SITE (1)..(10): subunit linker
LGGGSGGGGS

SEQ ID NO: 61 - protein, 10 aa, Artificial Sequence
  linker
  SITE (1)..(10): subunit linker
GLGGSGGGGS

SEQ ID NO: 62 - protein, 10 aa, Artificial Sequence
  linker
  SITE (1)..(10): subunit linker
GGLGSGGGGS

SEQ ID NO: 63 - protein, 10 aa, Artificial Sequence
  linker
  SITE (1)..(10): subunit linker
GGGLSGGGGS

SEQ ID NO: 64 - protein, 10 aa, Artificial Sequence
  linker
  SITE (1)..(10): subunit linker
GGGGLGGGGS

SEQ ID NO: 65 - protein, 10 aa, Artificial Sequence
  linker
  SITE (1)..(10): subunit linker
LGGSGGGGSG

SEQ ID NO: 66 - protein, 10 aa, Artificial Sequence
  linker
  SITE (1)..(10): subunit linker
GLGSGGGGSG

SEQ ID NO: 67 - protein, 10 aa, Artificial Sequence
  linker
  SITE (1)..(10): subunit linker
GGLSGGGGSG

SEQ ID NO: 68 - protein, 10 aa, Artificial Sequence
  linker
  SITE (1)..(10): subunit linker
GGGLGGGGSG

SEQ ID NO: 69 - protein, 10 aa, Artificial Sequence
  linker
  SITE (1)..(10): subunit linker
GGGSLGGGSG

SEQ ID NO: 70 - protein, 10 aa, Artificial Sequence
  linker
  SITE (1)..(10): subunit linker
LGGSSGGGSS

SEQ ID NO: 71 - protein, 10 aa, Artificial Sequence
  linker
  SITE (1)..(10): subunit linker
GLGSSGGGSS

SEQ ID NO: 72 - protein, 10 aa, Artificial Sequence
  linker
  SITE (1)..(10): subunit linker
GGLSSGGGSS

SEQ ID NO: 73 - protein, 10 aa, Artificial Sequence
  linker
  SITE (1)..(10): subunit linker
GGGLSGGGSS

SEQ ID NO: 74 - protein, 10 aa, Artificial Sequence
  linker
  SITE (1)..(10): subunit linker
GGGSLGGGSS

SEQ ID NO: 75 - protein, 10 aa, Artificial Sequence
  subunit linker
  SITE (1)..(10): subunit linker
LGGGSLGGGS

SEQ ID NO: 76 - protein, 10 aa, Artificial Sequence
  linker
  SITE (1)..(10): subunit linker
GLGGSGLGGS

SEQ ID NO: 77 - protein, 10 aa, Artificial Sequence
  linker
  SITE (1)..(10): subunit linker
GGLGSGGLGS

SEQ ID NO: 78 - protein, 10 aa, Artificial Sequence
  linker
  SITE (1)..(10): subunit linker
GGGLSGGGLS

SEQ ID NO: 79 - protein, 10 aa, Artificial Sequence
  linker
  SITE (1)..(10): subunit linker
GGGGLGGGGL

SEQ ID NO: 80 - protein, 10 aa, Artificial Sequence
  linker
  SITE (1)..(10): subunit linker
LGGSGLGGSG

SEQ ID NO: 81 - protein, 10 aa, Artificial Sequence
  linker
  SITE (1)..(10): subunit linker
GLGSGGLGSG

SEQ ID NO: 82 - protein, 10 aa, Artificial Sequence
  linker
  SITE (1)..(10): subunit linker
GGLSGGGLSG

SEQ ID NO: 83 - protein, 10 aa, Artificial Sequence
  linker
  SITE (1)..(10): subunit linker
GGGLGGGGLG

SEQ ID NO: 84 - protein, 10 aa, Artificial Sequence
  linker
  SITE (1)..(10): subunit linker
GGGSLGGGSL

SEQ ID NO: 85 - protein, 10 aa, Artificial Sequence
  linker
  SITE (1)..(10): subunit linker
LGGSSLGGSS

SEQ ID NO: 86 - protein, 10 aa, Artificial Sequence
  linker
  SITE (1)..(10): subunit linker
GLGSSGLGSS

SEQ ID NO: 87 - protein, 10 aa, Artificial Sequence
  linker
  SITE (1)..(10): subunit linker
GGLSSGGLSS

SEQ ID NO: 88 - protein, 6 aa, Artificial Sequence
  linker
  SITE (1)..(6): subunit linker
GSGGGA

SEQ ID NO: 89 - protein, 12 aa, Artificial Sequence
  linker
  SITE (1)..(12): subunit linker
GSGGGAGSGGGA

SEQ ID NO: 90 - protein, 18 aa, Artificial Sequence
  linker
  SITE (1)..(18): subunit linker
GSGGGAGSGGGAGSGGGA

SEQ ID NO: 91 - protein, 24 aa, Artificial Sequence
  linker
  SITE (1)..(24): subunit linker
GSGGGAGSGGGAGSGGGAGSGGGA

SEQ ID NO: 92 - protein, 10 aa, Artificial Sequence
  linker
  SITE (1)..(10): subunit linker
GENLYFQSGG

SEQ ID NO: 93 - protein, 10 aa, Artificial Sequence
  linker
  SITE (1)..(10): subunit linker
SGGGSSGGGS

SEQ ID NO: 94 - protein, 10 aa, Artificial Sequence
  linker
  SITE (1)..(10): subunit linker
SSGGGSSGGG

SEQ ID NO: 95 - protein, 10 aa, Artificial Sequence
  linker
  SITE (1)..(10): subunit linker
GGSGGGGSGG

SEQ ID NO: 96 - protein, 10 aa, Artificial Sequence
  linker
  SITE (1)..(10): subunit linker
GSGSGSGSGS

SEQ ID NO: 97 - protein, 6 aa, Artificial Sequence
  linker
  SITE (1)..(6): subunit linker
GGGSSS

SEQ ID NO: 98 - protein, 15 aa, Artificial Sequence
  linker
  SITE (1)..(15): subunit linker
GGGSSGGGSSGGGSS

SEQ ID NO: 99 - protein, 8 aa, Artificial Sequence
  linker
  SITE (1)..(8): subunit linker
GLGGLAAA

SEQ ID NO: 100 - protein, 12 aa, Artificial Sequence
  linker
  SITE (1)..(12): subunit linker
KPEPKPAPAPKP

SEQ ID NO: 101 - protein, 12 aa, Artificial Sequence
  linker
  SITE (1)..(12): subunit linker
AEAAAKEAAAKA

SEQ ID NO: 102 - protein, 5 aa, Artificial Sequence
  linker
  SITE (1)..(5): subunit linker
  SITE (1)..(5): the sequence can be repeated m times, where m is an integer from 1 to 5, e.g., 1, 2, 3, 4, or 5
EAAAK

SEQ ID NO: 103 - protein, 15 aa, Artificial Sequence
  linker
  SITE (1)..(15): subunit linker
PSRLEEELRRRLTEP

SEQ ID NO: 104 - protein, 8 aa, Artificial Sequence
  linker
  SITE (1)..(8): subunit linker
SACYCELS

SEQ ID NO: 105 - protein, 16 aa, Artificial Sequence
  linker
  SITE (1)..(16): subunit linker
TQKSLSLSPGKGLGGL

SEQ ID NO: 106 - protein, 13 aa, Artificial Sequence
  linker
  SITE (1)..(13): subunit linker
SLSLSPGKGLGGL

SEQ ID NO: 107 - protein, 36 aa, Artificial Sequence
  linker
  SITE (1)..(36): subunit linker
GGSAGGSGSGSSGGSSGASGTGTAGGTGSGSGTGSG

SEQ ID NO: 108 - protein, 36 aa, Artificial Sequence
  linker
  SITE (1)..(36): subunit linker
GGSGGGSEGGGSEGGGSEGGGSEGGGSEGGGSGGGS

SEQ ID NO: 109 - protein, 5 aa, Artificial Sequence
  linker
  SITE (1)..(5): subunit linker
GLSGL

SEQ ID NO: 110 - protein, 5 aa, Artificial Sequence
  linker
  SITE (1)..(5): subunit linker
EAAAK

SEQ ID NO: 111 - protein, 6 aa, Artificial Sequence
  linker
  SITE (1)..(6): subunit linker
  SITE (1)..(5): the sequence can be repeated m times, where m is an integer from 1 to 5, e.g., 1, 2, 3, 4, or 5
EAAKGS

SEQ ID NO: 112 - protein, 17 aa, Artificial Sequence
  linker
  SITE (1)..(17): subunit linker
GPSRLEEELRRRLTEPG

SEQ ID NO: 113 - DNA, 117 bp, Artificial sequence
  multimerisation unit derived from p53
aagcctctggacggagagtatttcactctccagatccggggccccgaaaggttcgaaatgttccgggagcttaacgaggccttggagctgaaagacgcacaggccggaaaggaaccg

SEQ ID NO: 114 - protein, 12 aa, Artificial Sequence
  linker
  SITE (1)..(12): subunit linker
HEYGAEALERAG

SEQ ID NO: 115 - DNA, 36 bp, Artificial Sequence
  ligand
  misc_feature (1)..(36): CLEC9 ligand
tggcccaggttccacagcagcgtgttccacacccac

SEQ ID NO: 116 - DNA, 180 bp, Artificial Sequence
  trimerization unit
  misc_feature (1)..(180): human collagen XV trimerization domain
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 117 - protein, 27 aa, Artificial Sequence
  subunit linker
  SITE (1)..(27): human collagen XV trimerization domain
GYIPEAPRDGQAYVRKDGEWVLLSTFL

SEQ ID NO: 118 - protein, 10 aa, Artificial Sequence
  dimerization unit linker
  SITE (1)..(10): dimerization unit linker
GGGSSGGGSG

SEQ ID NO: 119 - DNA, 333 bp, Artificial Sequence
  hinge region
  misc_feature (1)..(333): hinge region derived from IgM
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 120 - DNA, 105 bp, Artificial Sequence
  dHLX protein
  misc_feature (1)..(105): dHLX protein
ggagaactggaggaattacttaaacatctcaaggagttgctcaaaggccctaggaagggagaactggaggaactcctcaaacatctcaaggagttactaaaggga

SEQ ID NO: 121 - DNA, 324 bp, Mus musculus
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtct

SEQ ID NO: 122 - protein, 27 aa, Artificial Sequence
  neoantigen table 1
  SITE (1)..(27): Neoantigen
STMLYIRALKNPSLYGFSSGLNKDGIE

SEQ ID NO: 123 - protein, 27 aa, Artificial Sequence
  neoantigen table 1
  SITE (1)..(27): Neoantigen
GALKKLIYAAKLNTSLKALEGERNQVY

SEQ ID NO: 124 - protein, 27 aa, Artificial Sequence
  neoantigen table 1
  SITE (1)..(27): Neoantigen
DLVCDFQSFKYYAHATSLAGHLVSCPL

SEQ ID NO: 125 - protein, 27 aa, Artificial Sequence
  neoantigen table 1
  SITE (1)..(27): Neoantigen
EKLRNPCPNKEKAYQPPFAFRHVLKLT

SEQ ID NO: 126 - protein, 27 aa, Artificial Sequence
  neoantigen table 1
  SITE (1)..(27): Neoantigen
DTLSAMSNPRAMQVLLQIQQGLQTLAT

SEQ ID NO: 127 - protein, 27 aa, Artificial Sequence
  neoantigen table 1
  SITE (1)..(27): Neoantigen
GDVKIHAHKVVLANISPYFKAMFTGNL

SEQ ID NO: 128 - protein, 27 aa, Artificial Sequence
  neoantigen table 1
  SITE (1)..(27)
EVIQTSKYYMRDVIAIESAWLLELAPH

SEQ ID NO: 129 - protein, 27 aa, Artificial Sequence
  neoantigen table 1
  SITE (1)..(27): Neoantigen
FFSFFTHRFGHHVSPQVHIILANLYLL

SEQ ID NO: 130 - protein, 27 aa, Artificial Sequence
  neoantigen table 1
  SITE (1)..(27): Neoantigen
VILPQAPSGPSYATYLQPAQAQMLTPP

SEQ ID NO: 131 - protein, 27 aa, Artificial Sequence
  neoantigen table 1
  SITE (1)..(27): Neoantigen
LWVYLRPVPRPATIYLQILRLKPLTGE

SEQ ID NO: 132 - protein, 27 aa, Artificial Sequence
  neoantigen table 1
  SITE (1)..(27): Neoantigen
FVSPMAHYVPGIMAIESVVARFQFIVP

SEQ ID NO: 133 - protein, 27 aa, Artificial Sequence
  neoantigen table 1
  SITE (1)..(27): Neoantigen
TLAFLVLSTPAMFNRALKPFLKSCHFQ

SEQ ID NO: 134 - protein, 27 aa, Artificial Sequence
  neoantigen table 1
  SITE (1)..(27): Neoantigen
FLERPMDMPYMIFYPNNPLMTGQLLGS

SEQ ID NO: 135 - protein, 27 aa, Artificial Sequence
  neoantigen table 1
  SITE (1)..(27): Neoantigen
IPREVGDGTRVDPFPPVQTWMRLPKLV

SEQ ID NO: 136 - protein, 27 aa, Artificial Sequence
  neoantigen table 1
  SITE (1)..(27): Neoantigen
GSLFGSSRVQYVVNPAVKIVFLNIDPS

SEQ ID NO: 137 - protein, 27 aa, Artificial Sequence
  neoantigen table 1
  SITE (1)..(27): Neoantigen
NNLQKYIEIYVQKINPSRLPVVIGGLL

SEQ ID NO: 138 - protein, 27 aa, Artificial Sequence
  neoantigen table 1
  SITE (1)..(27): Neoantigen
AEYGDYQPEVHGVPYFRLEHYLPARVM

SEQ ID NO: 139 - protein, 27 aa, Artificial Sequence
  neoantigen table 1
  SITE (1)..(27): Neoantigen
TPLRKHTVHAIRKFYLEFKGSSPPPRL

SEQ ID NO: 140 - protein, 27 aa, Artificial Sequence
  neoantigen table 1
  SITE (1)..(27): Neoantigen
KIYEFDYHLYGQNITMIMTSVSGHLLA

SEQ ID NO: 141 - protein, 27 aa, Artificial Sequence
  neoantigen table 1
  SITE (1)..(27): Neoantigen
KSWIHCWKYLSVQSQLFRGSSLLFRRV